SEQUENCE LISTING (30 sequences)

SEQ ID NO: 1 - DNA, 996 bp, synthetic construct
  misc_feature 1..996: note = marB nucleotide sequence
  source 1..996: mol_type = other DNA
atgacggttcctgcttatccttcccgccagcctgcggccggcggagtttcatcttgcggtggcgcggggggcggctgcggggacaggacggcgtgcgacggcggcgacggcggtcgcgccaccgccccggtggtcgccctgcgcggtcgccatccctgcttcgaccccgccccccaggcccatgcccgggccgggcggctgcatctgccggtcagcccggcctgcaatatcacctgccagttctgcgcccgggatttcaacgcctccgaccgccgccccggcgtggcgcgccggcttctcaagcccgagcaagcccttgacgtggtgcgccgggcgctgcggctctgcccggaaatctcggtcgtcggcatcgccggccccggtgacactttggcgaccaatcacgccatcgacaccttcgccctgatccatgcggactttccgacgctgatcaactgcctgtcgaccaatggcctgcgcctgcccgatcgcgccaaggagctggccgccgttggtgttcagaccctgaccgtcaccgtcaatgccgtcgccccggagatccaggcggtgatttcgccggtgatcgccgatcgcggcaagcggctggagggtatcgaggcggcccgcgtgctgatcgccaaccagcttgagggcatcgccaaggcggtggctctcggcatggtggtcaaggtcaattgcgtgctgatccccggggtcaacgacgatcacatcggcgccgtcgcccaaaaagtggcggccgccggcgcctcgttgttcaacatcatcgccttgatccccacccataacctcgcccatctccccgcccccagcccggccctgctggcccgggcccagcgcgaggccggacgccacatcagcgtctttacccattgtcagcgctgccgcgccgatgccgccggcgtgcccggcgtcagcgatatcgccgacctgctttacgaccggcgtcttgacgccacgaccttttcccacggctag

SEQ ID NO: 2 - protein, 331 aa, synthetic construct
  REGION 1..331: note = MarB protein sequence
MTVPAYPSRQPAAGGVSSCGGAGGGCGDRTACDGGDGGRATAPVVALRGRHPCFDPAPQAHARAGRLHLPVSPACNITCQFCARDFNASDRRPGVARRLLKPEQALDVVRRALRLCPEISVVGIAGPGDTLATNHAIDTFALIHADFPTLINCLSTNGLRLPDRAKELAAVGVQTLTVTVNAVAPEIQAVISPVIADRGKRLEGIEAARVLIANQLEGIAKAVALGMVVKVNCVLIPGVNDDHIGAVAQKVAAAGASLFNIIALIPTHNLAHLPAPSPALLARAQREAGRHISVFTHCQRCRADAAGVPGVSDIADLLYDRRLDATTFSHG

SEQ ID NO: 3 - DNA, 876 bp, synthetic construct
  misc_feature 1..876: note = marH nucleotide sequence
  source 1..876: mol_type = other DNA
atggccaaaagtcccaaacaaatcgccatctatggcaaaggtggcatcggcaaatcgaccaccacctcgaatatcagcgccgccctggccgaggccggctacaaggtgatgcagttcggctgcgaccccaaaagcgattcgaccaataccctgcgcggcggcgattacatcccctcggtgctcgacctgctgcgcgagaacgcccgcgtcgatgcccatgaggcgatcttccagggctttggcggcatctattgcgttgaagccggtggtccggcgccaggcgtcggctgcgccggtcgcggcatcatcaccgccgtcgaactgctcaagcagcagaacgtcttcgaagagctcgatcttgattacgtgatcttcgacgtgctgggcgacgtggtctgcggcggcttcgccgtgccgatccgtgaaggcatcgccgaacatgtcttcaccgtgtcgtcgtcggatttcatggcgatctatgccgcgaacaatctgttcaagggcattcagaagtactccaacgccgggggcgccctgcttggcggggtgatcgccaattcgatcaacaccgatttccaccgggacatcatcgacgatttcgtcgcccgcacccagacccaggtcgtccaatacgtgccgcgctcgctgaccgtcacccaggccgaactgcagggccgcacgacgatcgaggcggcgcccgagtccgcccaggccgagatctatcggaccctggcgcgcagcatcgccgaccatacggactcgaaggtgccgaccccgcttaacgcccaagagctgcgcgactggtcggcatcctgggccaaccaattgatcgagatcgaacgggcgagccagccgattcccgccctggcctcataa

SEQ ID NO: 4 - protein, 291 aa, synthetic construct
  REGION 1..291: note = MarH protein sequence
MAKSPKQIAIYGKGGIGKSTTTSNISAALAEAGYKVMQFGCDPKSDSTNTLRGGDYIPSVLDLLRENARVDAHEAIFQGFGGIYCVEAGGPAPGVGCAGRGIITAVELLKQQNVFEELDLDYVIFDVLGDVVCGGFAVPIREGIAEHVFTVSSSDFMAIYAANNLFKGIQKYSNAGGALLGGVIANSINTDFHRDIIDDFVARTQTQVVQYVPRSLTVTQAELQGRTTIEAAPESAQAEIYRTLARSIADHTDSKVPTPLNAQELRDWSASWANQLIEIERASQPIPALAS

SEQ ID NO: 5 - DNA, 1536 bp, synthetic construct
  misc_feature 1..1536: note = marD nucleotide sequence
  source 1..1536: mol_type = other DNA
atgcccatcaatctcaagacatcggtggtcgagagccgcgaacagcggctgggcaccatcatcgcctgggacggcaaggcctctgacctgtccaaggaatcggcctatgcgcgcagcgagggctgcggcagcgcctgcggcgccaaggcccgccgggtctgcgagatgcgcagcccgttcagccagggctcggtctgtagcgaacagatggtcgaatgccaagccggcaacgtgcgcggcgccgtgctggtccagcattcgccgatcggctgcggcgccggtcaggtgatctataattcgatcttccgcaatggtctggcgatccgcggcctgccggtggagaacctccatctgatcagcaccaacctgcgcgaacgcgacatggtctatggcgggctcgacaagctcgaacgcaccatccgcgacgcctgggagcgccatcacccccaggccattttcatcgccacctcctgcccgacggcgatcattggcgacgacatcgaaagcgtcgcttcgcagcttgaagccgagttcggcataccggtcataccgctgcactgcgagggcttcaaatccaagcattggagcaccggcttcgacgccacccagcacggcatcttgcgccagatcgtccgcaaaaatcccgagcgcaagcaggaagacctggtcaacgtcatcaatctgtggggatcggatgtctttggcccgatgctcggcgaattgggtttgcgggtgaactacgtcgtcgatctcgccaccgtcgaggatctggcccagatgtcggaggcggcggcaaccgtcggcttctgctacacgctgtcgacctatatggccgccgccctggaacaggaattcggcgttcccgaggtcaaggcgcccatgccctatggcttcgccggcaccgacgcctggctgcgcgagatcgcccgcgtcacccaccgcgaggagcaggccgaggcctatatcgcccgcgagcacgcccgggtgaagccacagcttgaggccctgcgcgagaagctcaagggcatcaagggcttcgtctccaccggctcggcctatgcccatggcatgatccaggtgctgcgcgaactgggcgtcaccgtcgacggctcgttggtcttccaccacgatccggtctacgacagccaggatccgcgtcaggattcccttgcccatctggtcgacaactatggcgacgtcggccatttcagcgtcggcaatcgccagcagttccagttctacggcctgcttcagcgggtgaagcccgatttcatcatcatccgccacaacgggttggcgccgctggcctcgcgcctgggcatcccggccattccgctgggcgatgaacatatcgccgtgggctatcagggcatcttgaacctgggtgaatccatcctcgatgtgctggcccaccgcaagttccacgaagacatcgccgcccatgtccgcctgccctatcgccaggactggctggcccgcgatcccttcgatctggcccggcaaagcgccggccagccgcgccgtcccgcagagtga

SEQ ID NO: 6 - protein, 511 aa, synthetic construct
  REGION 1..511: note = MarD protein sequence
MPINLKTSVVESREQRLGTIIAWDGKASDLSKESAYARSEGCGSACGAKARRVCEMRSPFSQGSVCSEQMVECQAGNVRGAVLVQHSPIGCGAGQVIYNSIFRNGLAIRGLPVENLHLISTNLRERDMVYGGLDKLERTIRDAWERHHPQAIFIATSCPTAIIGDDIESVASQLEAEFGIPVIPLHCEGFKSKHWSTGFDATQHGILRQIVRKNPERKQEDLVNVINLWGSDVFGPMLGELGLRVNYVVDLATVEDLAQMSEAAATVGFCYTLSTYMAAALEQEFGVPEVKAPMPYGFAGTDAWLREIARVTHREEQAEAYIAREHARVKPQLEALREKLKGIKGFVSTGSAYAHGMIQVLRELGVTVDGSLVFHHDPVYDSQDPRQDSLAHLVDNYGDVGHFSVGNRQQFQFYGLLQRVKPDFIIIRHNGLAPLASRLGIPAIPLGDEHIAVGYQGILNLGESILDVLAHRKFHEDIAAHVRLPYRQDWLARDPFDLARQSAGQPRRPAE

SEQ ID NO: 7 - DNA, 1401 bp, synthetic construct
  misc_feature 1..1401: note = marK nucleotide sequence
  source 1..1401: mol_type = other DNA
atgcccgatgcagagtcccgttcccaggtcacggcgaaggccgcgccaccacccgcccccaagaccaattcgatcgaacaggtgcgctatatctgttcgatcggcgccatgcacagcgcctcggctatcccacgggtgatcccgatcacccattgcggcccgggctgcgccgacaagcagttcatgaacgtcgccttctataatggcttccagggcggcggctatggcggcggagcggtggtgccgagcaccaacgccaccgagcgcgaggtggtcttcggcggcgccgagcgcctggacgaattgatcggcgcctcgctgcaggtgcttgacgccgacctgttcgtggtgctgaccggctgtattcccgatctggtcggcgatgacatcggctcggtggtcggcccctatcagaagcgcggcgtgccgatcgtctatgccgagactggcggctttcgcggcaataacttcaccggccacgaactggtgaccaaggcgatcatcgaccagttcgttggcgattacgatgcggagcgcgacggggcccgcgagccccatacggtcaatgtctggtcactgctgccctaccacaacaccttctggcgcggtgatttgaccgagatcaagcggctgctcgaaggcatcggccttaaggtcaatatcctgttcggcccgcaatcggccggggtggcggaatggaaggccatcccgcgcgccggctttaatctggtgctctcgccctggctggggctggacacggcgcgccatttggaccgcaaatacggccagccgaccctgcatcgaccgatcatcccgatcggcgccaaggaaaccggcgccttcctgcgcgaggtggcggctttcgccggcctcgacagcgcggtggtcgaggccttcatcaccgccgaagaagccgtttattaccgctatctggaggacttcaccgatttctacgcggagtactggtggggtctgccggccaaattcgccgtcatcggcgacagcgcctataatctggccttgaccaaattcctggtaaaccagttgggcctgataccggggctgcagatcatcaccgacaatccgcccgaggaggtgcgcgaggatatccgcgcccattaccacgcgatcgccgatgacgtggccaccgatgtctcttttgaagaagacagctacaccatccaccaaaagatccgcgccaccgatttcggccacaaggcgccgatcctgtttggcaccacctgggaacgcgaccttgccaaggaattgaagggggcgatcgtcgaggtcggcttcccggcatcctatgaagtcgtgctgtcgcgcagctatcttggctaccggggcgccctgactttgctggaaaaaatctacacaaccaccgtcagcgcaagcgcttga

SEQ ID NO: 8 - protein, 465 aa, synthetic construct
  REGION 1..465: note = MarK protein sequence
PDAESRSQVTAKAAPPPAPKTNSIEQVRYICSIGAMHSASAIPRVIPITHCGPGCADKQFMNVAFYNGFQGGGYGGGAVVPSTNATEREVVFGGAERLDELIGASLQVLDADLFVVLTGCIPDLVGDDIGSVVGPYQKRGVPIVYAETGGFRGNNFTGHELVTKAIIDQFVGDYDAERDGAREPHTVNVWSLLPYHNTFWRGDLTEIKRLLEGIGLKVNILFGPQSAGVAEWKAIPRAGFNLVLSPWLGLDTARHLDRKYGQPTLHRPIIPIGAKETGAFLREVAAFAGLDSAVVEAFITAEEAVYYRYLEDFTDFYAEYWWGLPAKFAVIGDSAYNLALTKFLVNQLGLIPGLQIITDNPPEEVREDIRAHYHAIADDVATDVSFEEDSYTIHQKIRATDFGHKAPILFGTTWERDLAKELKGAIVEVGFPASYEVVLSRSYLGYRGALTLLEKIYTTTVSASA

SEQ ID NO: 9 - DNA, 179 bp, synthetic construct
  misc_feature 1..179: note = Promoter derived from Rhodospirillum rubrum mtnP promoter
  source 1..179: mol_type = other DNA
aaaccgctttaaccgccatcctgcgctaaacggccgccggcccccaccggcggccgttttttattcgccgcccctccccgcgacgggctccctcgccttggtggcttttcatccgggggggtggcgcgctaaggtgccccacccgcaaaagggtgagccagccaggaagaggggaacat

SEQ ID NO: 10 - DNA, 216 bp, synthetic construct
  misc_feature 1..216: note = Promoter derived from Rhodospirillum rubrum puf promoter
  source 1..216: mol_type = other DNA
gggcatggcgcggatgatccgcccgctctcgggctcgccacacgaggttttccggggttttccgctcctttcggggcagaacacgccggataacaaggtccgtcccgacctggtcgggtggacttcttaccgcggttcttcaccgcggtagagcagccgttccctgcgcggatgcagtggaatggttttctgggcaagaattaggaggtagcacat

SEQ ID NO: 11 - DNA, 40 bp, synthetic construct
  misc_feature 1..40: note = R1066 forward primer
  source 1..40: mol_type = other DNA
gacggtgtggaggatcccatggagtggtacattgactcgg

SEQ ID NO: 12 - DNA, 37 bp, synthetic construct
  misc_feature 1..37: note = R1066 reverse primer
  source 1..37: mol_type = other DNA
cctgcccgtctagaatggttatccgctcgatcatcgg

SEQ ID NO: 13 - DNA, 38 bp, synthetic construct
  misc_feature 1..38: note = R1069 forward primer
  source 1..38: mol_type = other DNA
ccatagcggactagtcaattacgtcaaccgtatcggcg

SEQ ID NO: 14 - DNA, 36 bp, synthetic construct
  misc_feature 1..36: note = R1069 reverse primer
  source 1..36: mol_type = other DNA
ccgccgcttgcatgcaaacgccttgatcctcaaggc

SEQ ID NO: 15 - DNA, 35 bp, synthetic construct
  misc_feature 1..35: note = R0793 forward primer
  source 1..35: mol_type = other DNA
ctgtttcaggatcctgggtccgacggtactctatc

SEQ ID NO: 16 - DNA, 41 bp, synthetic construct
  misc_feature 1..41: note = R0793 reverse primer
  source 1..41: mol_type = other DNA
cctgacttttctagaaaaaatctacacaaccaccgtcagcg

SEQ ID NO: 17 - DNA, 38 bp, synthetic construct
  misc_feature 1..38: note = R0796 forward primer
  source 1..38: mol_type = other DNA
gaaactccgactagtgcaggctggcgggaaggataagc

SEQ ID NO: 18 - DNA, 35 bp, synthetic construct
  misc_feature 1..35: note = R0796 reverse primer
  source 1..35: mol_type = other DNA
gcgcaagggcatgccgttgtccatcgtgtatggcg

SEQ ID NO: 19 - DNA, 35 bp, synthetic construct
  misc_feature 1..35: note = R0772 forward primer
  source 1..35: mol_type = other DNA
caaaggtggatccacaacgccactttatcctccgc

SEQ ID NO: 20 - DNA, 36 bp, synthetic construct
  misc_feature 1..36: note = R0772 reverse primer
  source 1..36: mol_type = other DNA
cggctgtttctagacgccatcacccacaaactccag

SEQ ID NO: 21 - DNA, 33 bp, synthetic construct
  misc_feature 1..33: note = R0773 forward primer
  source 1..33: mol_type = other DNA
cgtcgttcgactagttcgaccggctggagcggc

SEQ ID NO: 22 - DNA, 32 bp, synthetic construct
  misc_feature 1..32: note = R0773 reverse primer
  source 1..32: mol_type = other DNA
ccgtatcggcatgccaacccaggacgcctttg

SEQ ID NO: 23 - DNA, 40 bp, synthetic construct
  misc_feature 1..40: note = C0796 forward primer
  source 1..40: mol_type = other DNA
ggagacggctcatatgacggttcctgcttatccttcccgc

SEQ ID NO: 24 - DNA, 30 bp, synthetic construct
  misc_feature 1..30: note = C0796 reverse primer
  source 1..30: mol_type = other DNA
gatgggcatggtacccgttatgaggccagg

SEQ ID NO: 25 - DNA, 40 bp, synthetic construct
  misc_feature 1..40: note = C0794 forward primer 1
  source 1..40: mol_type = other DNA
cggagcggccatatgcccatcaatctcaagacatcggtgg

SEQ ID NO: 26 - DNA, 30 bp, synthetic construct
  misc_feature 1..30: note = C0793 reverse primer
  source 1..30: mol_type = other DNA
ggcggcctcgagcccggatgccgccattcc

SEQ ID NO: 27 - DNA, 37 bp, synthetic construct
  misc_feature 1..37: note = C0794 forward primer 2
  source 1..37: mol_type = other DNA
cggagcggtaccatgcccatcaatctcaagacatcgg

SEQ ID NO: 28 - DNA, 30 bp, synthetic construct; identical to SEQ ID NO: 26
  misc_feature 1..30: note = C0793 reverse primer
  source 1..30: mol_type = other DNA

SEQ ID NO: 29 - DNA, 42 bp, synthetic construct
  misc_feature 1..42: note = C0773 forward primer
  source 1..42: mol_type = other DNA
ggaggcgggtaccgtgacaaagatcgaaaagccgctccagcc

SEQ ID NO: 30 - DNA, 36 bp, synthetic construct
  misc_feature 1..36: note = C0772 reverse primer
  source 1..36: mol_type = other DNA
catcacccctcgagccacaccgggcgaccgcacagc

(19) United States
(12) Patent Application Publication
(10) Pub. No.: US 2024/0060037 A1
(43) Pub. Date: Feb. 22, 2024

(54) MODIFIED ORGANISMS FOR ETHYLENE, ETHANE, AND METHANE BIOGENESIS AND METHODS FOR USE THEREOF

(71) Applicant: Ohio State Innovation Foundation, Columbus, OH (US)

(72) Inventors: Justin NORTH, Columbus, OH (US); Srividya MURALI, Dublin, OH (US); Sarah YOUNG, Gahanna, OH (US); Fred TABITA, (US)

(21) Appl. No.: 18/473,637

(22) Filed: Sep. 25, 2023

Related U.S. Application Data
(63) Continuation of application No. PCT/US2022/021905, filed on Mar. 25, 2022.
(60) Provisional application No. 63/165,904, filed on Mar. 25, 2021.

(51) Int. Cl. C12N 1/20; C12N 9/02; C12N 15/63; C12P 5/02; C12N 9/10; C12N 9/88

(52) U.S. Cl. CPC C12N 1/205; C12N 9/0095; C12N 15/63; C12P 5/02; C12N 9/1007; C12N 9/88; C12Y 404/01011; C12Y 201/01251; C12Y 118/00; C12R 2001/01

(57) ABSTRACT

The present disclosure provides non-naturally occurring microbial organisms capable of producing ethylene, ethane, and/or methane, as well was methods for producing ethylene, ethane, and/or methane using the same.

### CROSS-REFERENCE TO RELATED APPLICATIONS

This application claims the benefit of priority to U.S. Provisional Application No. 63/165,904, filed Mar. 25, 2021, the disclosure of which is incorporated herein by reference in its entirety,

### STATEMENT REGARDING FEDERALLY SPONSORED RESEARCH OR DEVELOPMENT

This invention was made with government support under Grant No. DE-SC0019338 awarded by the U.S. Department of Energy. The Government has certain rights in the invention.

### BACKGROUND

Nitrogenases are an ancient group of enzymes, existing approximately 3.2 billion years ago, before the evolution of oxygenic photosynthesis and subsequent widespread oxygenation (1, 2), Their essential function is reduction of dinitrogen gas into ammonia, contributing over half of the annual global nitrogen fixation required for the synthesis of nucleic and amino acids by all life on earth (3). Ancestors to nitrogenase in anaerobic prokaryotes also gave rise to distinct nitrogenase-like reductases for bacterial photosynthesis and archaeal methanogenesis cofactor metabolism (4, 5, 6, 7). These include the dark operative protochlorophyllide oxidoreductase (DPOR) and chlorophyllide α oxidoreductase (COR) of bacteriochlorophyll biosynthesis, and Ni²⁺-sirohydrochlorin a,c-diamide reductive cyclase for biosynthesis of the archaeal methyl coenzyme-M reductase cofactor F430 (4, 5, 6, 7). However, the evolutionary history of nitrogen fixation revealed overlooked nitrogen fixation-like (NFL) sequences in the genomes of anaerobic bacteria with entirely unknown function. Some were surprisingly associated with sulfur metabolism and transport genes (8, 9). This suggested that certain members of the nitrogenase family potentially have a role in sulfur metabolism.

Previous production of ethylene gas (>1 μmol/h/g dry cell weight) was observed from photosynthetic Alphaproteobacteria such as Rhodospirillum rubrum and Rhodopseudomonas palustris when growing anaerobically under the low sulfate concentrations (<200 μM) commonly encountered in their freshwater and soil habitats (see FIGS. 5A-C) (10). The precursor of ethylene, (2-methylthio)ethanol (MT-EtOH), and the pathway for its production was documented (P2017-343-096 Novel Microbial Process to Synthesize Ethylene) (10). This volatile organic sulfur compound (VOSC) was produced from byproducts of S-adenosyl-L-methionine (SAM) utilization to regenerate methionine (FIG. 1A; DHAP shunt) (10). SAM is a key cellular cofactor synthesized directly from methionine and is required by all organisms for diverse processes including DNA, RNA, and protein methylations, polyamine and neurotransmitter synthesis, quorum sensing, and 5′-deoxyadenosyl radical generation by radical SAM enzymes (11). However, the enzymes responsible for the liberation of sulfur from MT-EtOH for methionine regeneration and concomitant ethylene formation were unresolved (10). These enzymes are thus disclosed to be a reductase of the nitrogenase-like family of enzymes, specifically a methylthio-alkane reductase (Mar) composed of components MarB, MarH, MarD, and MarK (MarBHDK) (see FIG. 1A).

There is a clear need for methods of producing the industrial precursor compounds ethylene, ethane, and methane, and microorganisms for the same. In particular, known ethylene producing enzyme systems require oxygen (aminocyclopropanecarboxylate oxidase and 2-oxoglutatate dioxygenase), forming a flammable ethylene-oxygen gas mixture. In addition methane and ethane when mixed with air are also explosive and flammable. Therefore, a microorganism and enzyme system to produce significant levels of ethylene, ethane, or methane in the absence of oxygen would have great utility.

### SUMMARY

The present disclosure provides non-naturally occurring microbial organisms which are capable of producing ethylene, ethane, methane, or combinations thereof.

In one aspect, a non-naturally occurring microbial organism is provided comprising a nucleic acid encoding one or more genes of a methylthio-alkane reductase complex and one or more genes of a methionine salvage pathway.

In another aspect, a non-naturally occurring microbial organism is provided, wherein the organism is an anaerobic organism which produces ethylene, ethane, and/or methane using a methylthio-alkane reductase complex and a methionine salvage pathway, and wherein the organism has been optimized for producing ethylene, ethane, and/or methane with one or more non-naturally occurring genes.

In another aspect, a method of producing ethylene, ethane, and/or methane is provided, the method comprising:

culturing a population of the non-naturally occurring microbial organism described herein in a culture medium comprising one or more carbon sources; and

recovering the ethylene, ethane, and/or methane.

A bioreactor is further provided comprising the non-naturally occurring microbial organism described herein.

A vector is also provided comprising: one or more exogenous nucleic acid molecules encoding one or more genes of a methylthio-alkane reductase complex and one to or more genes of a methionine salvage pathway.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1C show that nitrogenase-like proteins are linked to VOSC utilization. (FIG. 1A) Methylthio-alkane reductase (1), the gene product of marBHDK (proposed), converts VOSCs to ethylene, methane, and methanethiol for methionine biosynthesis. MT-EtOH is produced by the widespread DHAP shunt (10, 25, 26) (FIG. 1B) R. rubrum proteins with increased abundance when methylthio-alkane reductase activity is induced (“MT-EtOH” or “Lo” 50 μM sulfate) versus repressed (“Hi” 1 mM sulfate), X; isolated Tn5 transposon mutants (FIG. 7), which could not utilize MT-EtOH for growth. (FIG. 1C) Changes in gene transcript abundance of R. rubrum parent strain (WRdht) and SalR deletion strain (0785::Tn5) under “Hi” and “Lo” sulfate. *; no significant change, p>0.25, two-tailed; Enzyme and Compound Key: 1) methylthio-alkane reductase; 2) serine/homoserine O-acetyltransferase (2.3.1.31); 3) O-acetylhomoserine sulfhydrylase (2.5.1.49); 4) S-adenosylmethionine synthetase (2.1.1.13, 2.1.1.14); 5) methionine synthase (2.5.1.6); 6) cystathionine beta-synthase (4.2.1.22); 7) cystathionine gamma-lyase (4.4.1.1); 8) cystathionine gamma-synthase (2.5.1.48); cystathionine beta-lyase (4.4.1.8); 10) MTA nucleosidase (3.2.2.16); 11) 5-methylthioribose kinase (2.7.1.100); 12) MTA phosphorylase (2.4.2.28); 13) 5-methylthioribose-1-phosphate isomerase (5.3.1.33); 14) 5-methylthioribulose-1-phosphate aldolase (4.1.2.n); 15) alcohol dehydrogenase (1.1.1.1); DHAP) dihydroxyacetone phosphate; HSE) homoserine; OAHS) O-acetyl-L-homoserine; SRH) S-ribosyl-L-homocysteine; R-H) methyl acceptor; THF) tetrahydrofolate.

FIGS. 2A-2F show that genes for marBHDK are required for anaerobic methionine metabolism from VOSCs. (FIG. 2A) Growth and average total hydrocarbon production of strains utilizing sulfate or VOSCs (see FIG. 8 and FIG. 19). *; not applicable. (FIG. 2B) Total amount of hydrocarbons produced when cells were fed with the indicated VOSC. (FIG. 2C) Plasmid-based complementation studies of NFL genes for growth of R. rubrum NFL gene deletion strain. (A-C) error bars are standard deviation for N=3 independent biological replicates. (FIGS. 2D and 2E) Identification of methionine (RT=8.5 min) and methanethiol (RT=28.3 min) upon feeding R. rubrum strains with (2-[methyl-C¹⁴]thio)ethanol (RT=22.8 min) (FIG. 2F) Change in Gibbs free energy under standard. conditions for the conversion of VOSCs to methanethiol and the corresponding hydrocarbon. H₂ represents 2e⁻ and 2H⁺ equivalents.

FIGS. 3A-3D show that methylthio-alkane reductase and nitrogenase are independent. (FIGS. 3A and 3B) Stoichiometric production of methane and ethane by cells feed with DMS and EMS. (FIG. 3C) Competition assays for methylthio-alkane reductase repression in cells grown with 1 mM MT-EtOH or DMS plus the indicated amount of sulfate. Non-linear fit to the Hill equation gives EC50_{DMS/sulfate}=140 μM sulfate and EC50_{MT-EtOH/sulfate}=110 μM sulfate for 50% activity with DMS and MT-EtOH as substrate, respectively. (FIG. 3D) Whole-cell methylthio-alkane reductase (Mar) and molybdenum nitrogenase (NifHDK) activities for wild type (WT) and Δ0772:3/Δ0793:6 deletion (ΔΔ) strains under methylthio-alkane reductase inducing (50 μM sulfate) or repressing (1 mM sulfate) and NifHDK inducing (Glu, glutamate) or repressing (NH₄⁺, ammonium) conditions. Standard deviations are (A-C) the error bars or (D) are <10% for N=3 biological replicates.

FIG. 4 shows that methylthio-alkane reductases are phylogenetically distinct. Phylogenetic tree of NifD superfamily homologues. The scale bar represents the number of substitutions per site. Nodes with UFBoot support values ≥95% indicated with black circles. Clade labeling: Group IV-A (NfaD; nitrogen fixation IV-A) (27), Group IV-B (CfbD; Ni2+-sirohydrochlorin a,c-diamide reductive cyclase) (4,5), Group IV-C (MarD; putative methylthio-alkane reductase), Group IV and Group VI (NfID; nitrogen fixation-like of unknown function), Group V (ChlN; DPOR, and BchY; COR). Clade labels and colors are per Raymond (9) and Méheust (28). Av, Azotobacter vinelandii; Bv, Blastochloris viridis; Ep, Endomicrobium proavitum; Rc, Rhodobacter capsulatus, Rp, Rhodopseudomonas palustris; Ru, Rhodospirillum rubrum.

FIGS. 5A-5C show the ethylene specific rate of production during growth. Bacteria growth measured via optical density at 660 nm (O.D. 660 nm) and the corresponding specific rate of ethylene production in μmol ethylene per hour per gram dry cell weight for (FIGS. 5A and 5B) Limiting sulfate concentrations and (FIG. 5C) MT-EtOH (200 μM). Error bars are the standard deviation for N=3 independent growth experiments (biological replicates).

FIG. 6 shows the Rhodospirillum rubrum thiol cluster. Known sulfur metabolism genes (yellow) and other genes of putative and unknown function, potentially involved in sulfur metabolism, are localized to a cluster of genes in the R. rubrum genome. This region contains the Group IV-C NFL genes marBHDK required for methylthio-alkane reductase activity and the Group IV NFL genes nflDK of unknown function (red).

FIGS. 7A-7B show the Rhodaspirillum rubrum strain WRdht(ΔrlpA::Gm^{R}/Δαld2) transposon mutagenesis screen. (FIGS. 7A, 7B, and 7C) Example of screen and identification of a random R. rubrum Tn5 transposon mutant (isolate 17E5) that is incapable of growing on MT-EtOH but retains capability for growing on sulfate. These mutants presumably are defective in metabolism specific to MT-EtOH and are selected for further growth and sequencing analysis as summarized in panel (FIG. 7D).

FIGS. 8A-8F show the growth of R. rubrum wild type and deletion strains. Culture optical density measured at 660 nm (O.D. 660 nm) for cells in the absence of a sulfur source (none) or 1 mM of the indicated sulfur source. Error bars are the standard deviation for N=3 independent growth experiments (biological replicates).

FIGS. 9A-9C show the substrate variability and complementation growth studies of R. rubrum. (FIG. 9A) Screen for R. rubrum growth with additional VOSCs and sulfur-containing amino acids. Each sulfur source was supplied at 1 mM concentration. Error bars are the standard deviation for N=3 independent growth experiments (biological replicates). (FIGS. 9B and 9C) Plasmid-based complementation of NFL genes for growth of R. rubrum NFL gene deletion strain (strain Δ0772:3/Δ0793:6) utilizing 1 mM sulfate or 1 nM DMS, respectively, as sole sulfur source. Error bars are the standard deviation for N=4 independent growth experiments (biological replicates).

FIG. 10 shows the NifD superfamily amino acid alignment. Pairwise alignment of NifD superfamily sequences are shown in the region of active site residues responsible for coordination of the P-cluster and FeMo-cofactor within the molybdenum nitrogenase subunit NifD (*), and substrate bound to the FeMo-cofactor (▾). Numbering is based off of Azotobacter vinelandii NifD (Av) (9).

FIG. 11 shows the NifK superfamily amino acid alignment. Pairwise alignment of NifK superfamily sequences are shown in the region of active site residues responsible for coordination of the P-cluster within the molybdenum nitrogenase subunit NifK (*). Numbering is based off of Azotobacter vinelandii NifK (Av) (9). Note that the group IV-B nitrogenase-like Ni2+-sirohydrochlorin a,c-diamide reductive cyclase (CfbCD) do not contain a NifK counterpart.

12 shows the NifH superfamily ammo acid alignment. Pairwise alignment of NifH superfamily sequences are shown in the region of active site residues responsible for MgATP binding and hydrolysis Fe₄—S₄ iron sulfur cluster binding (*). The conserved arginine (▾) is the site of ADP-ribosylation post translational modification for nitrogenase activity regulation in the bona fide nitrogenases. ADP-ribosylation performed by dinitrogenase reductase ADP-ribosyl transferase (DraT) in R. rubrum prevents association of NifH with NifDK. The modification is removed by dinitrogenase reductase activating glycohydrolase (DraG). Numbering is based off of Azotobacter vinelandii NifH (Av) (9). For NflH, NfaH, and MarH, corresponding genes were located with 10 genes upstream or downstream from nflDK, nfαKD, , and marDK, respectively, in each organism.

FIG. 13 shows the NifB superfamily amino acid alignment. Pairwise alignment of NifB superfamily sequences are shown in the regions of conservation for molybdenum nitrogenase NifB sequences. The Radical SAM motif CxxxCxxC cysteines (*) coordinates the Fe₄—S₄ cluster responsible for binding S-adenosyl-1-methionine (SAM). The SAM methyl group provides the carbide during formation of the NifB-cofactor precursor to FeMo-, FeV-, or FeFe-cofactor. Numbering is based off of Azotobacter vinelandii NifB (Av) (12). Note that the group IV-B Ni2+-sirohydrochtorin a,c-diamide reductive cyclases (CfbCD) and group V bacteriochlorophyll reductases DPOR (ChlLNB) and COR (BchXYZ) do not require or posess a NifB counterpart for assembly. For NflB, NfaB, and MarB, corresponding genes, if present, were located with 10 genes upstream or downstream from nflDK, nfαDK, and marDK, respectively, in each organism.

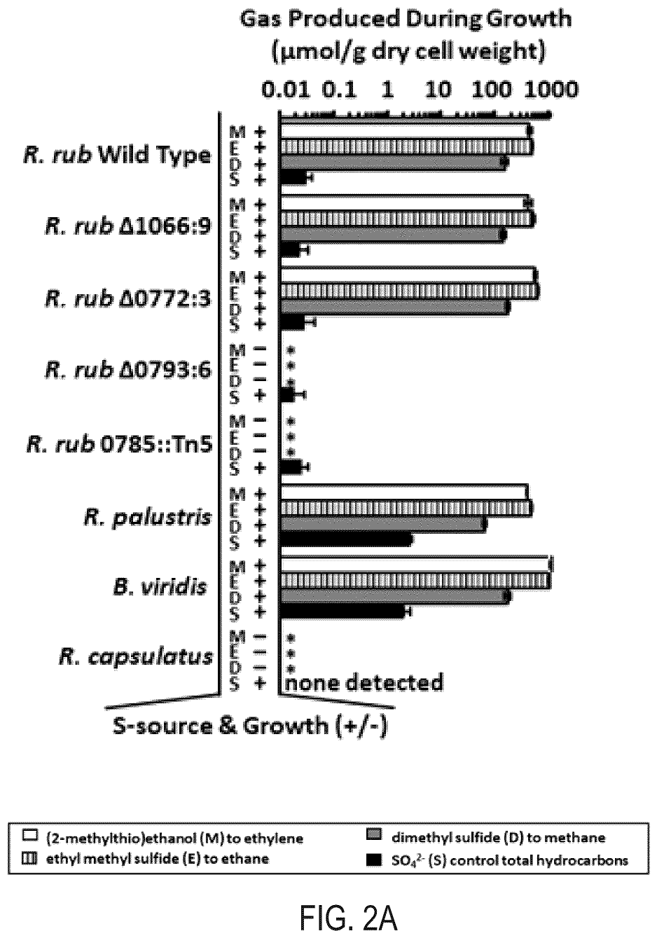

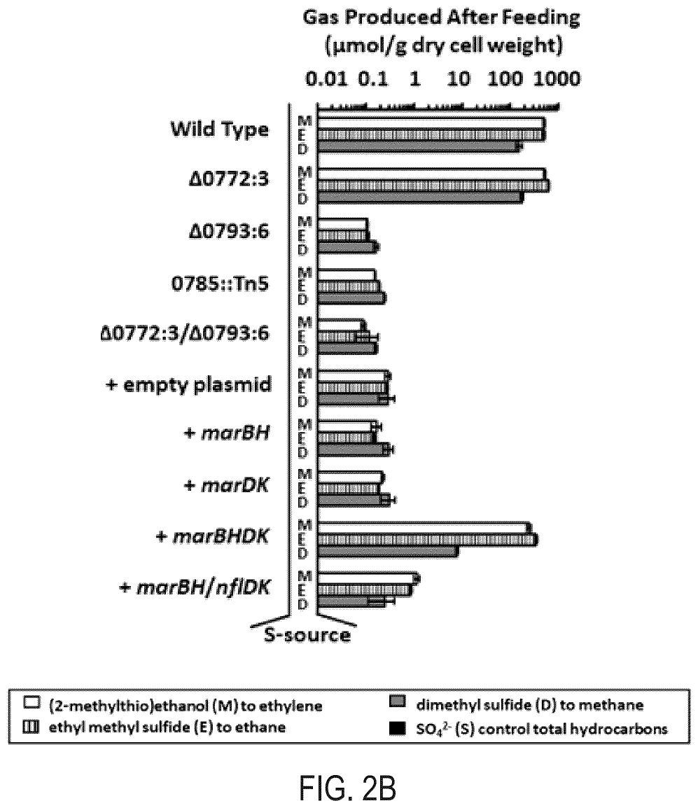

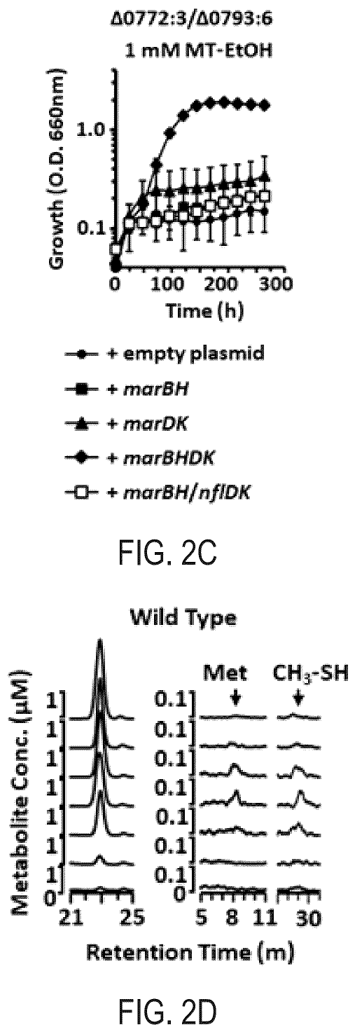

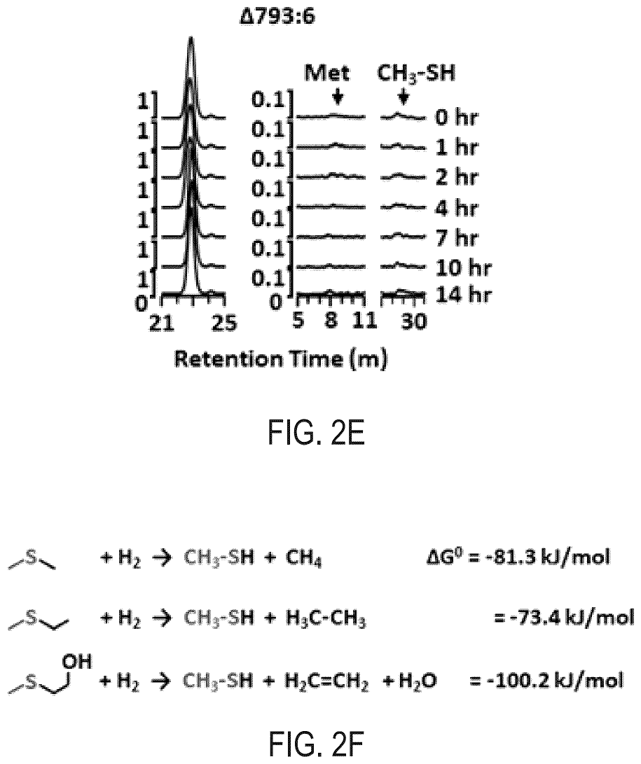

FIGS. 14A-14B show total C¹⁴ incorporation from (2-[methyl-C¹⁴]thio)ethanol. The wild type strain (FIG. 14A) and Δ793:6 marBHDK deletion strain (FIG. 14B) was fed with (2-[methyl-C¹⁴]thio)ethanol for the indicated amount of time. The radioactivity present due to soluble metabolites in the extracellular media and extracted from the cells was measured by scintillation counting before resolving metabolites by HPCL (FIG. 2D-E). The remaining insoluble material present in the cells was coordinately measured by scintillation counting, which indicates the amount of C¹⁴ incorporation into cell material via methionine synthesis. The total radioactivity is the sum of the soluble and insoluble components. Data is a representative C¹⁴ incorporation series for N=2 independent feeding experiments (biological replicates).

FIGS. 15A-15B show the thermodynamics of ethanol versus ethylene and water formation from MT-EtOH. (FIG. 15A) Thermodynamic cycle comparing the formation of ethanol to the formation of ethylene from MT-EtOH. The difference in the formation free energies can be understood in terms of ΔG₃=ΔG₂−ΔG₁. (FIG. 15B) Detailed thermodynamics in which it can be seen that in reaction 3, the gas phase reaction energy ΔEᵣₓₙ(g) favors ethanol formation from ethylene and water by −52.3 kJ/mol, but even in the gas phase the reaction is entropically disfavored due to the loss of degrees of freedom in going from two molecules to one, resulting in ΔGᵣₓₙ(g)=7.1 kJ/mol. Then, the free energy of solvation of ethanol is less favorable than the solvation of the ethylene and water by 11.0 kJ/mol. Although the COSMO solvation model does not account explicitly for hydrogen bonding (which would likely favor the reactants, as well), solvating the water dipole and ethylene quadrapole pair are likely more favorable than solvating the single ethanol dipole. The combined effect of entropy loss and differences in solvation is to make ethanol formation unfavorable relative to ethylene and water by 18.1 kJ/mol.

FIG. 16 shows NifH superfamily phylogenetic analysis. Phylogenetic tree of NifH superfamily homologues based on an LG+R10 evolution model. The scale bar represents the number of substitutions per site. UFBoot support values of 95% or greater are shown as black circles on branches (56). For disambiguation, enzymes of known function are labeled Group IV-A (NfaH, nitrogen fixation IV-A) (27), Group IV-B (CfbC; Ni2+-sirohydrochlorin a,c-diamide reductive cyclase) (4,5), and Group IV-C (MarH; putative methylthio-alkane reductase). Group IV and Group VI NifH homologues of unknown function are designated NW Group V is Ch (DPOR) and BchX. (COR). Ciade coloting follows Raymond (9) and Méheust (28). Av, Azotobacter vinelandii; Bv, Blastochloris viridis; Ep, Endomicrobium proavitum; Rc, Rhodobacter capsulatus; Rp, Rhodopseudomonas palustris; Ru, Rhodospirillum rubrum.

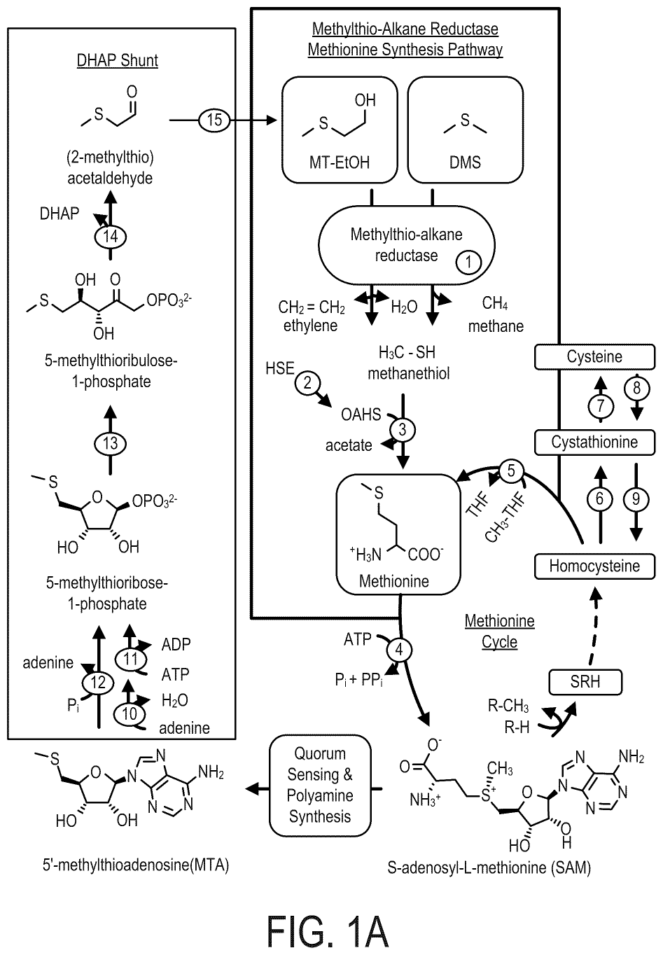

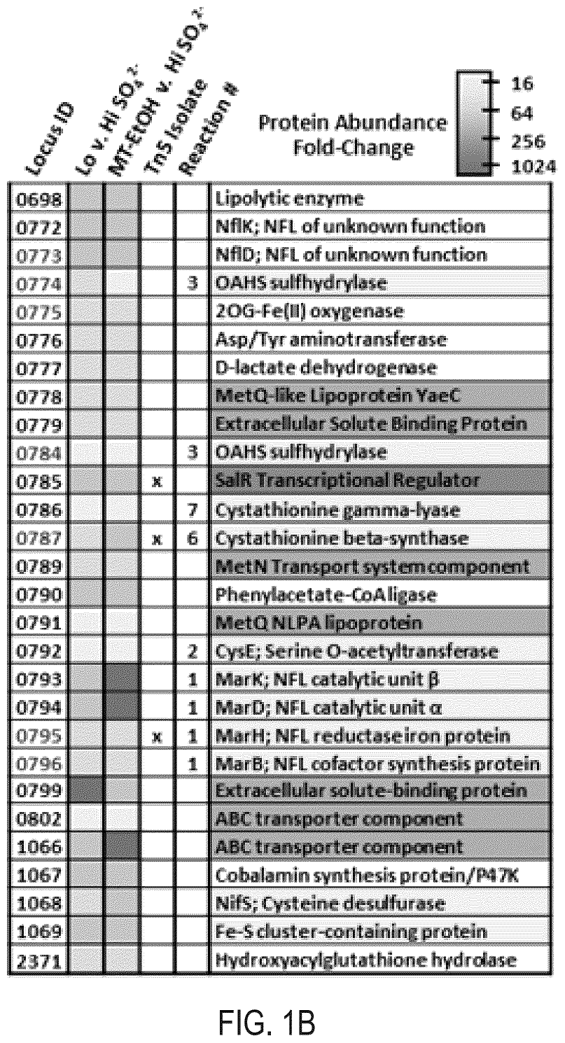

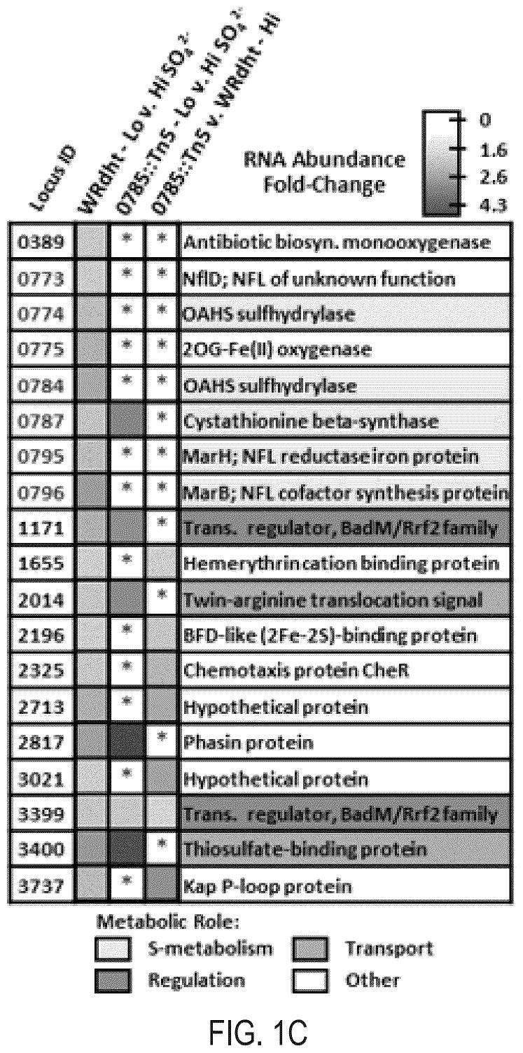

FIG. 17 shows organisms with genes for 5′-methylthioadenosine salvage via DHAP Shunt and methylthio-alkane reductase pathways. The DHAP shunt for conversion of 5′-methylthioadenosine to MT-EtOH is composed of MTA phosphorylase (MtnP) or 5-methylthioribose kinase (MtnK) and 5-methylthioribose-1-phosphate isomerase (MtnA) and 5-methylthioribulose-1-phosphate aldolase (Ald2) (see FIG. 1A). Black circles represent UFBoot bootstrap values of 100. Nodes are labeled to indicate phylum membership.

FIG. 18 shows the genes and their putative functions surrounding Group IV and VI gene clusters. Homologous sulfur metabolism genes, which are enriched in Groups IV-A, IV-C, and IV of unknown function, are indicated as described in the key.

FIGS. 19A-19C show the identification of methylthio-alkane reductase capabilities in other alpha-proteobacteria. Culture optical density measured at 660 nm (O.D. 660 nm) for cells in the absence of a sulfur source (none) or 1 mM of the indicated sulfur source. Error bars are the standard deviation for N=3 independent growth experiments (biological replicates). Blastochloris viridis DSM 133 and Rhodopseudomonas palustris CGA010 possess marBHDK homologues, whereas Rhodobacter capsulatus SB1003 does not.

FIGS. 20A-20D show the methionine Salvage Pathways for ethylene and methane, optimization, and bioreactor design. The bioreactor design employs cellulolytic bacteria to convert corn stover biomass into industrially tractable gases (ethylene and methane) utilizing a novel anaerobic methionine salvage pathway discovered in certain photosynthetic bacteria and clostridia. (FIG. 20A) Bioreactor design for conversion of cellulosic biomass to ethylene and methane biogas. (FIG. 20B) Methionine salvage pathway and ethylenelethanehnethane producing enzyme system (MarBHDK) for biogas production. (FIG. 20C) Example of pathway construction in cellulose degrading Bacilli and Clostridia for production of ethylene and methane. (FIG. 20D) Optimization of ethylene production using non-naturally occurring gene from Coliphage, SAM hydrolase, for direct conversion of SAM to MTA.

Like reference symbols in the various drawings indicate like elements,

### DETAILED DESCRIPTION

Methane is used for the production of energy, hydrogen gas, synthesis gas, and methanol used in the manufacturing of various organic chemicals. Methane is the second most used energy source next to electricity. Ethylene is used in a variety of industrial processes, including the production of polyethylene for plastic bags, polystyrene for packaging and insulation, and ethylene oxide for detergents. In addition, ethylene may be converted to C5-C10 gasoline-like molecules. Ethylene is thus thought to be the most widely used chemical on earth (over 175 million tons in 2018) and the demands and market for this feedstock are steadily increasing, with nearly a $300 billion annual market. Thus, there is considerable interest in developing new and innovative ways to produce these key industrial precursor compounds (ethylene, ethane, methane) with bio-based methods as a potential way to supplement chemical-based processes.

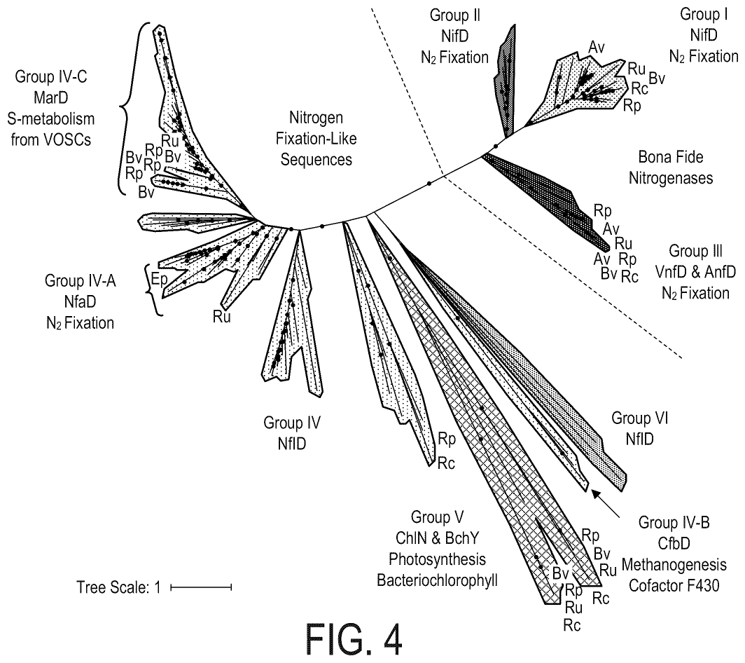

For anaerobic ethylene production by microorganisms, the novel and widespread bacterial carbon and sulfur salvage pathway, the DHAP Shunt (FIG. 1A), converts the ubiquitous S-adenosyl-L-methionine byproduct, MTA, into adenine, DHAP, and the volatile organic sulfur compound, (2-methylthio)ethanol (MT-EtOH). This includes freshwater and soil bacteria such as Rhodospirillum rubrum and Rhodopseudomonas palustris, extra-intestinal pathogenic Escherichia coli, and pathogenic Bacillus species (10, 25, 26, 67). It was demonstrated that the Alphaproteobacteria, R. rubrum and R. palustris, were able to further utilize MT-EtOH as a sole sulfitr source for growth and synthesis of sulfur-containing amino acids (e.g. methionine), producing stoichiometric amounts of ethylene gas in the process (10). This process was strictly anaerobic and clearly enzymatic in nature (10). This was the first reported solely anaerobic route to ethylene, and involves a novel cooperation of genes and enzymes (MarBHDK). It was subsequently found that the enzyme system producing ethylene from MT-EtOH (MarBHDK) was a member of the nitrogenase family of enzymes from a novel and distinct Glade (FIG. 4 and FIG. 16). This strictly anaerobic methylthio-alkane reductase system not only could product ethylene form MT-EtOH, but it could also produce ethane from ethylmethylsulfide (CH₃—S—CH₂—CH₃) and methane from dimethylsulfide (CH₃—S—CH₃). This was verified in alphaproteobacteria, including Rhodopseudomonas plaustris, Rhodospirillum rubrum, and Blastochloris viridis. A search of the available database for other organisms that possess the same set of discovered genes encoding nitrogenase-like methylthio-alkane reductase enzymes for reactions for ethylene, ethane, and methane formation indicated that this enzyme was prevalent in genomes from multiple phyla of industrially relevant Proteobacteria and Firmicutes. It was also found that these genes were detected in anoxic high carbon ecosystems including wetland soils and animal rumen. Notably, expressed proteins for methylthio-alkane reductase were recovered in situ, supporting the ability to use a functional screen to potentially recover catalytically active enzymes from the environment.

Disclosed herein is an exclusively anaerobic enzyme system and associated pathways that couples sulfur metabolism to ethylene and methane production in the purple non-sulfur alpha-proteobacteria. Rhodospirillum rubrum, Rhodopseudomonas palustris, and Blastochloris viridis (FIGS. 1A-1C). Genes for this anaerobic enzyme system are widely distributed amongst bacteria (FIG. 17), and this pathway reveals a possible route by which ethylene and methane, both of which are frequently observed in anoxic environments, can be produced by indigenous microbes.

Disclosed herein are methods for the development of a potential industrially compatible process to biologically produce ethylene and methane in high yields. Disclosed herein is a method to fully characterize the anaerobic ethylenelethane/methane producing enzyme system and determine how the genes are regulated at the molecular level. Computational modeling of the chemical reactions performed by the relevant enzymes are initiated to learn the mechanisms by which these enzymes catalyze the reactions involved in ethylene biosynthesis. In addition, since ethylene/ethane/methane synthesis from the respective precursor compound is an inducible process, further studies probe the molecular regulation of the genes involved during photosynthetic metabolism using a variety of “omics” tools. These biochemical and molecular studies are invaluable for optimizing ethylene/ethane/methane production and creating bacterial strains that over-produce ethylene/ethane/methane under controlled conditions.

Also disclosed herein is a method to maximize ethylene and methane production with different feedstocks; e.g., lignocellulose digests as well as inorganic carbon sources (FIGS. 20A-20D). Rps. palustris, as well as cellulolytic and acetogenic bacteria such as Ruminiclostridium josui and Clostridium ljungdhalii species all contain the genes for the ethylene/ethane/methane producing enzyme system MarBHDK (FIG. 17), and each of these organisms has the capacity to grow on cellulosic digests as well as inorganic carbon sources (CO₂). Conditions are optimized for each of these growth conditions.

Further disclosed are metagenomics and bioinformatic/computational approaches to discover more effective enzymes of uncultured organisms from anaerobic environments. Analysis of existing genome and metagenome databases allow identification of potential gene sequences for ethylene/ethane/methane producing enzymes systems that have specific or enhanced catalytic properties. Such sequences, homologous to known genes, may then be screened for their effectiveness in catalyzing key reactions of ethylene/ethane/methane synthesis. This leverages over 4 billion years of evolution to obtain the most efficient enzymes. In addition, a functional genomics approach may be established to isolate relevant genes from the metagenome without previous knowledge of sequences; e.g., by complementing specific mutant host organisms with environmental DNA (68). These metagenomics approaches, plus a full battery of other synthetic biology and “omics” approaches is utilized to optimize ethylene/ethane/methane formation.

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the terns “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated. range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “culture”, “cultivate”, and “ferment” are used interchangeably and refer to the intentional growth, propagation, proliferation, and/or enablement of metabolism, catabolism, and/or anabolism of one or more cells (e.g. a microbial organism). The combination of both growth and propagation may be termed proliferation, Examples include production by an organism of ethylene, ethane, or methane. Culture does not refer to the growth or propagation of microorganisms in nature or otherwise without human intervention.

The term “growth” means an increase in cell size, total cellular contents, and/or cell mass or weight of a cell (e.g. a microbial organism).

A “growth media” or “growth medium” as used herein can be a solid, powder, or liquid mixture which comprises all or substantially all of the nutrients necessary to support the growth of microbial organisms; various nutrient compositions are preferably prepared when particular microbial species are being assayed. Amino acids, carbohydrates, minerals, vitamins and other elements known to those skilled in the art to be necessary for the growth of microbial organisms are provided in the medium. In one embodiment, the growth medium is liquid.

The term “propagation” refers to an increase in cell number via cell division.

The term “promoter” or “regulatory element” refers to a region or sequence determinants located upstream or downstream from the start of transcription and which are involved in recognition and binding of RNA polymerase and other proteins to initiate transcription. Promoters need not be of origin in the microbial organism used, for example, promoters derived from viruses or from other organisms can be used in the compositions or methods described herein,

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y. (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide).

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones. The term encompasses nucleic acids containing known analogues of natural nucleotides which have similar or improved binding properties, for the purposes desired, as the reference nucleic acid. A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of splice variants is discussed in Leicher, et al., J. Biol. Chem. 273 (52):35095-35101 (1998).

The term “expression cassette” refers to a nucleic acid construct, which when introduced into a host cell, results in transcription and/or translation of a RNA or polypeptide, respectively. In some embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.hih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPS containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH The Tₘ is the temperature (under defined ionic strength, pH, and nucleic concentration) at which 50% of the probes complementary to the target hybridize to the target sequence at equilibrium the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al. One of skill will recognize that these values can be appropriately adjusted to determine corresponding identity of proteins encoded by two nucleotide sequences by taking into account codon degeneracy, amino acid similarity, reading frame positioning and the like. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

The term “modulator” refers to a composition that increases or decreases the level of a target molecule or the level of activity or function of a target molecule or the physical state of the target of the molecule. In embodiments a modulator is a recombinant nucleic acid that is capable of increasing or decreasing the amount of a protein in a cell or the level of activity of a protein in a cell or transcription of a second nucleic acid in a cell. In embodiments, a modulator increases or decreases the level of activity of a protein or the amount of the protein in a cell. The term “modulate” is used in accordance with its plain and ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, a recombinant nucleic acid that modulates the level of activity of a protein may increase the activity or amount of the protein relative the absence of the recombinant nucleic acid. In embodiments, an increase in the activity or amount of a protein may include overexpression of the protein. “Overexpression” is used in accordance with its plain and ordinary meaning and refers to an increased level of expression of a protein relative to a control (e.g. cell or expression system not including a recombinant nucleic acid that contributes to the overexpression of a protein). In embodiments, a decrease in the activity or amount of a protein may include a mutation (e.g. point mutant, loss of function mutation, missense mutation, deletion, or insertion of heterologous nucleic acid; all/any of which may be in the coding region for a protein or in an operably linked region (e.g, promoter)) of the protein. The term “increased” refers to a detectable increase compared to a control.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

Transformation” refers to the transfer of a nucleic acid molecule into a host organism (e.g. a microbial organism). In embodiments, the nucleic acid molecule may be a plasmid that replicates autonomously or it may integrate into the genome of the host organism (e.g. a microbial organism). Host organisms containing the transformed nucleic acid molecule may be referred to as “transgenic” or “recombinant” or “transformed” organisms (e.g. microbial organisms). A “genetically modified” organism (e.g. genetically modified microbial organism) is an organism (e.g. microbial organism) that includes a nucleic acid that has been modified by human intervention. Examples of a nucleic acid that has been modified by human intervention include, but are not limited to, insertions, deletions, mutations, expression nucleic acid constructs (e.g. over-expression or expression from a non-natural promoter or control sequence or an operably linked promoter and gene nucleic acid distinct from a naturally occurring promoter and gene nucleic acid in an organism), extra-chromosomal nucleic acids, and genomically contained modified nucleic acids. Genetically modified organisms may be made by rational modification of a nucleic acid or may be made by use of a mutagen or mutagenesis protocol that results in a mutation that was not identified (e.g. intended or targeted) prior to the use of the mutagen or mutagenesis protocol (e.g. UV exposure, EMS exposure, mutagen exposure, random genomic mutagenesis, transformation of a library of different nucleic acid constructs). Genetically modified organisms that include a modification (e.g. modification, insertion, deletion, mutation) not previously known or intended prior to making of the genetically modified organism may be identified through screening a plurality of organism including one or more genetically modified organisms by using a selection criteria that identifies the genetically modified organism of interest. In embodiments, a genetically modified organism includes a recombinant nucleic acid.

As used herein, the term “episome” or “episomally” is intended to refer to an extrachromosomal DNA moiety or plasmid that can replicate autonomously in a host cell when physically separated from the chromosomal DNA of the host cell.

Methods for synthesizing sequences and bringing sequences together are well established and known to those of skill in the art. For example, in vitro mutagenesis and selection, site-directed mutagenesis, error prone PCR (Melnikov et al., Nucleic Acids Research, 27 (4)1056-1062 (Feb. 15, 1999)), “gene shuffling” or other means can be employed to obtain mutations of naturally occurring genes.

### Compositions

### Microbial Organisms

The present disclosure provides non-naturally occurring microbial organisms which are capable of producing ethylene, ethane, methane, or combinations thereof. some aspects, the microbial organism has been genetically modified with one or more genes directed to the production of ethylene, ethane, methane, or combinations thereof. In other aspects, the microbial organism may naturally produce ethylene, ethane, methane, or combinations thereof, but has been optimized for said production by the introduction of one or more non-naturally occurring genes.

Thus, in one aspect, a non-naturally occurring microbial organism is provided comprising a nucleic acid encoding one or more genes of a methylthio-alkane reductase complex and one or more genes of a methionine salvage pathway.

In some embodiments, the organism can produce ethylene, ethane, methane, or combinations thereof, In some embodiments, the organism produces ethylene, In some embodiments, the organism produces ethane. In some embodiments, the organism produces methane.

In another aspect, a non-naturally occurring microbial organism is provided, wherein the organism is an anaerobic organism which produces ethylene, ethane, and/or methane using a methylthio-alkane reductase complex and a methionine salvage pathway, and wherein the organism has been optimized for producing ethylene, ethane, and/or methane with one or more non-naturally occurring genes. In some embodiments, the one or more non-naturally occurring genes comprise one or more genes of a SAM hydrolase. In some embodiments, the one or more non-naturally occurring genes comprise one or more genes of a methanethiol methylase (mddik), a methionine gamma lyase (mgt), or combinations thereof.

### Methylthio-Alkane Reductases

In some embodiments, the one or more genes of a methylthio-alkane reductase complex may comprise marB, marH, marD, marK, or combinations thereof.

In some embodiments, the one or more genes of a methylthio-alkane reductase complex comprise marB. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 1 (marB).

SEQ ID NO: 1
ATGACGGTTCCTGCTTATCCTTCCCGCCAGCCTGCGGCCG
GCGGAGTTTCATCTTGCGGTGGCGCGGGGGGCGGCTGCGG
GGACAGGACGGCGTGCGACGGCGGCGACGGCGGTCGCGCC
ACCGCCCCGGTGGTCGCCCTGCGCGGTCGCCATCCCTGCT
TCGACCCCGCCCCCCAGGCCCATGCCCGGGCCGGGCGGCT
GCATCTGCCGGTCAGCCCGGCCTGCAATATCACCTGCCAG
TTCTGCGCCCGGGATTTCAACGCCTCCGACCGCCGCCCCG
GCGTGGCGCGCCGGCTTCTCAAGCCCGAGCAAGCCCTTGA
CGTGGTGCGCCGGGCGCTGCGGCTCTGCCCGGAAATCTCG
GTCGTCGGCATCGCCGGCCCCGGTGACACTTTGGCGACCA
ATCACGCCATCGACACCTTCGCCCTGATCCATGCGGACTT
TCCGACGCTGATCAACTGCCTGTCGACCAATGGCCTGCGC
CTGCCCGATCGCGCCAAGGAGCTGGCCGCCGTTGGTGTTC
AGACCCTGACCGTCACCGTCAATGCCGTCGCCCCGGAGAT
CCAGGCGGTGATTTCGCCGGTGATCGCCGATCGCGGCAAG
CGGCTGGAGGGTATCGAGGCGGCCCGCGTGCTGATCGCCA
ACCAGCTTGAGGGCATCGCCAAGGCGGTGGCTCTCGGCAT
GGTGGTCAAGGTCAATTGCGTGCTGATCCCCGGGGTCAAC
GACGATCACATCGGCGCCGTCGCCCAAAAAGTGGCGGCCG
CCGGCGCCTCGTTGTTCAACATCATCGCCTTGATCCCCAC
CCATAACCTCGCCCATCTCCCCGCCCCCAGCCCGGCCCTG
CTGGCCCGGGCCCAGCGCGAGGCCGGACGCCACATCAGCG
TCTTTACCCATTGTCAGCGCTGCCGCGCCGATGCCGCCGG
CGTGCCCGGCGTCAGCGATATCGCCGACCTGCTTTACGAC
CGGCGTCTTGACGCCACGACCTTTTCCCACGGCTAG

In some embodiments, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 1 (marB). In some embodiments, the one or more genese of a methylthio-alkane reductase complex comprise a nucleic acid sequence of SEQ ID No: 1.

In some embodiments, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 2 (MarB).

SEQ ID NO: 2
MTVPAYPSRQPAAGGVSSCGGAGGGCGDRTACDGGDGGRA
TAPVVALRGRHPCFDPAPQAHARAGRLHLPVSPACNITCQ
FCARDENASDRRPGVARRLLKPEQALDVVRRALRLCPEIS
VVGIAGPGDTLATNHAIDTFALIHADFPTLINCLSTNGLR
LPDRAKELAAVGVQTLTVTVNAVAPEIQAVISPVIADRGK
RLEGIEAARVLIANQLEGIAKAVALGMVVKVNCVLIPGVN
DDHIGAVAQKVAAAGASLFNIIALIPTHNLAHLPAPSPAL
LARAQREAGRHISVFTHCQRCRADAAGVPGVSDIADLLYD
RRLDATTFSHG

In some embodiments, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 2. In some embodiments, the one or more genes of a methylthio-alkane reductase complex comprise a gene encoding a protein of SEQ ID NO: 2. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments, the one or more genes of a methyltbio-alkane reductase complex comprise marH. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 3 (marH).

SEQ ID NO: 3
ATGGCCAAAAGTCCCAAACAAATCGCCATCTATGGCAAAG
GTGGCATCGGCAAATCGACCACCACCTCGAATATCAGCGC
CGCCCTGGCCGAGGCCGGCTACAAGGTGATGCAGTTCGGC
TGCGACCCCAAAAGCGATTCGACCAATACCCTGCGCGGCG
GCGATTACATCCCCTCGGTGCTCGACCTGCTGCGCGAGAA
CGCCCGCGTCGATGCCCATGAGGCGATCTTCCAGGGCTTT
GGCGGCATCTATTGCGTTGAAGCCGGTGGTCCGGCGCCAG
GCGTCGGCTGCGCCGGTCGCGGCATCATCACCGCCGTCGA
ACTGCTCAAGCAGCAGAACGTCTTCGAAGAGCTCGATCTT
GATTACGTGATCTTCGACGTGCTGGGCGACGTGGTCTGCG
GCGGCTTCGCCGTGCCGATCCGTGAAGGCATCGCCGAACA
TGTCTTCACCGTGTCGTCGTCGGATTTCATGGCGATCTAT
GCCGCGAACAATCTGTTCAAGGGCATTCAGAAGTACTCCA
ACGCCGGGGGCGCCCTGCTTGGCGGGGTGATCGCCAATTC
GATCAACACCGATTTCCACCGGGACATCATCGACGATTTC
GTCGCCCGCACCCAGACCCAGGTCGTCCAATACGTGCCGC
GCTCGCTGACCGTCACCCAGGCCGAACTGCAGGGCCGCAC
GACGATCGAGGCGGCGCCCGAGTCCGCCCAGGCCGAGATC
TATCGGACCCTGGCGCGCAGCATCGCCGACCATACGGACT
CGAAGGTGCCGACCCCGCTTAACGCCCAAGAGCTGCGCGA
CTGGTCGGCATCCTGGGCCAACCAATTGATCGAGATCGAA
CGGGCGAGCCAGCCGATTCCCGCCCTGGCCTCATAA

In some embodiments, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 3. In some embodiments, the one or more genese of a methylthio-alkane reductase complex comprise a nucleic acid sequence of SEQ ID NO: 3.

In some embodiments, the one or more genes of a methylthio-alkane reductase complex comprise one or more marll genes associated with an accession number found in Table 1 below:

TABLE 1
Representative MarIl Genes

| Whole Genome Sequence of Origin | Accession Number |
|---|---|
| Pararhodospirillum oryzae strain NBRC 107573 sequence093, | WP_147164651.1 |
| whole genome shotgun sequence | |
| Rhodospirillum photometricum DSM 122 draft genome sequence | WP_041796112.1 |
| Rhodospirillum rubrum ATCC 11170 chromosome, complete | YP 425886.1 |
| genome | |
| Rhodospirillum rubrum F11, complete genome | WP_011388553.1 |
| Phaeospirillum fulvum MGU-K5 contig00054, whole genome | WP_021132881.1 |
| shotgun sequence | |
| Rhodoblastus sphagnicola strain DSM 16996 scaffold0018, whole | WP_104506083.1 |
| genome shotgun sequence | |
| Rhodoblastus acidophilus strain DSM 137 | WP_088522711.1 |
| NODE 116 length 9951 cov_47.3758, whole genome shotgun | |
| sequence | |
| Rhodoblastus acidophilus strain DSM 137, whole genome shotgun | WP_088522711.1 |
| sequence | |
| Rhodoblastus acidophilus strain DSM 137 scaffold0022, whole | WP_088522711.1 |
| genome shotgun sequence | |
| Rhodomicrobium sp. JA980 | WP_127076529.1 |
| NODE 3 length 364448_cov_26.852217, whole genome shotgun | |
| sequence | |
| Pleomorphomonas carboxyditropha strain SVCO-16 | WP_100079641.1 |
| NODE 2 length 581917_cov_22.4871, whole genome shotgun | |
| sequence | |
| Rhodomicrobium vannielii ATCC 17100, complete genome | WP_013421120.1 |
| Thermoanaerobacterium thermosaccharolyticum M0795, complete | WP_041589431.1 |
| genome | |
| Bacteroidales bacterium Barb6XT Barb6XT contig 167, whole | WP_066182115.1 |
| genome shotgun sequence | |
| Prevotella bryantii strain TC1-1 contig9, whole genome shotgun | WP_006281725.1 |
| sequence | |
| Selenomonas ruminantium strain WCT3, whole genome shotgun | WP_033169720.1 |
| sequence | |
| Selenomonas sp. ND2010 T504DRAFT scaffold00003.3_C, whole | WP_033169720.1 |
| genome shotgun sequence | |
| Phaeospirillum fulvum strain DSM 13234, whole genome shotgun | WP_074764655.1 |
| sequence | |
| Clostridium coskatii strain PTA-10522 CLCOS_contig000056, | WP_063601658.1 |
| whole genome shotgun sequence | |
| Clostridium coskatii strain PS02 scaffold19_1_86601, whole | WP_063601658.1 |
| genome shotgun sequence | |
| Clostridium autoethanogenum strain H21-9 Contig_058, whole | WP_013239001.1 |
| genome shotgun sequence | |
| Clostridium ljungdahlii DSM 13528 strain PETC | WP_013239001.1 |
| scaffold3 200123 404054, whole genome shotgun sequence | |
| Clostridium drakei strain SLI contig_79, whole genome shotgun | WP_032079660.1 |
| sequence | |
| Clostridium drakei strain SLI chromosome, complete genome | WP_032079660.1 |
| Clostridium scatologenes strain ATCC 25775, complete genome | WP_029160437.1 |
| Fibrobacter sp. UWT2, whole genome shotgun sequence | WP_072801408.1 |
| Fibrobacter sp. UWB8, whole genome shotgun sequence | WP_072977618.1 |
| Fibrobacter sp. UWB6 Ga0136278_108, whole genome shotgun | WP_072977618.1 |
| sequence | |
| Fibrobacter sp. UWB15, whole genome shotgun sequence | WP_072977618.1 |
| Fibrobacter sp. UWBS NODE_1, whole genome shotgun sequence | WP_072977618.1 |
| Fibrobacter sp. UWB1 NODE_4, whole genome shotgun sequence | WP_073321569.1 |
| Fibrobacter sp. UWOVI, whole genome shotgun sequence | WP_073321569.1 |
| Fibrobacter sp. UWH4, whole genome shotgun sequence | WP_072977618.1 |
| Selenomonas bovis 8-14-1 T485DRAFT_scaffold00002.2_C, whole | WP_031584323.1 |
| genome shotgun sequence | |
| Rhodopseudomonas palustris strain 2.1.37 scaffold_36, whole | WP_011158185.1 |
| genome shotgun sequence | |
| Rhodopseudomonas palustris strain DSM 126 scaffold0001, whole | WP_011158185.1 |
| genome shotgun sequence | |
| Rhodopseudomonas palustris strain RI | WP_119018516.1 |
| NODE_28_length_158663_cov_40.885563, whole genome shotgun | |
| sequence | |
| Rhodopseudomonas sp. AAP120 AAP120_Contigs_11, whole | WP_054160731.1 |
| genome shotgun sequence | |
| Fibrobacter succinogenes subsp. succinogenes S85, complete | WP_014545823.1 |
| genome | |
| Fibrobacter succinogenes subsp. succinogenes S85, complete | WP_014545823.1 |
| genome | |
| Blastochloris viridis genome assembly Blastochloris viridis genome, | WP_055037160.1 |
| chromosome : I | |
| Blastochloris viridis strain ATCC 19567, complete genome | WP_055037160.1 |
| Blastochloris viridis DNA, complete genome, strain: DSM 133 | WP_055037160.1 |
| Clostridium autoethanogenum DSM 10061 seq4, whole genome | WP_013239001.1 |
| shotgun sequence | |
| Clostridium autoethanogenum strain JA1-1 | WP_013239001.1 |
| scaffold2 136726 570037, whole genome shotgun sequence | |
| Ruminococcaceae bacterium HV4-5-B5C, whole genome shotgun | WP_114174611.1 |
| sequence | |
| Clostridium bornimense replicon M2/40_rep1, complete genome, | WP_044035927.1 |
| type strain M2/40T | |
| Clostridium ljungdahlii strain ERI-2 scaffold7, whole genome | WP_063557083.1 |
| shotgun sequence | |
| Clostridium chromiireducens strain DSM 23318 | WP_079439996.1 |
| CLCHR contig000029, whole genome shotgun sequence | |
| Rhodopseudomonas palustris strain R1 | WP_119017311.1 |
| NODE_7_length_89266_cov_41.693230, whole genome shotgun | |
| sequence | |
| Rhodopseudomonas palustris strain DSM 126 scaffold0020, whole | WP_011157906.1 |
| genome shotgun sequence | |
| Rhodopseudomonas palustris strain 2.1.37 scaffold_3, whole | WP_011157906.1 |
| genome shotgun sequence | |
| Pleomorphomonas carboxyditropha strain SVCO-16 | WP_100081799.1 |
| NODE_13_length_137005_cov_21.4606, whole genome shotgun | |
| sequence | |
| Pleomorphomonas sp. CF100 Ga0189743_114, whole genome | WP_134185339.1 |
| shotgun sequence | |
| Pleomorphomonas koreensis DSM 23070 | WP_026783525.1 |
| H512DRAFT_scaffold00010.10_C, whole genome shotgun | |
| sequence | |
| Roseiarcus fermentans strain DSM 24875 Ga0244512_102, whole | WP_113887556.1 |
| genome shotgun sequence | |
| Ruminococcus flavefaciens strain XPD3002, whole genome shotgun | WP_075423707.1 |
| sequence | |
| Clostridium beijerinckii HUN142 T483DRAFT_scaffold00009.9_C, | WP_026886176.1 |
| whole genome shotgun sequence | |
| Clostridium beijerinckii strain NRRL B-591 CLBKI_contig000007, | WP_011967971.1 |
| whole genome shotgun sequence | |
| Clostridium beijerinckii strain 4J9 CLOSB_contig000013, whole | WP_011967971.1 |
| genome shotgun sequence | |
| Clostridium beijerinckii ATCC 35702, complete genome | WP_011967971.1 |
| Clostridium beijerinckii NCIMB 8052, complete genome | WP_011967971.1 |
| Clostridium beijerinckii G117 Scaffold22, whole genome shotgun | WP_017212486.1 |
| sequence | |
| Clostridium beijerinckii strain WB | WP_017212486.1 |
| Clostridium beijerinckii_WB_contig15, whole genome shotgun | |
| sequence | |
| Clostridium beijerinckii strain DSM 791 CLBEI_contig000075, | WP_011967971.1 |
| whole genome shotgun sequence | |
| Clostridium beijerinckii strain NBRC 109359 sequence070, whole | WP_011967971.1 |
| genome shotgun sequence | |
| Clostridium beijerinckii strain BAS/B2 CLBEJ_contig000034, | WP_011967971.1 |
| whole genome shotgun sequence | |
| Clostridium beijerinckii strain NCP 260 CLOBJ_contig000033, | WP_011967971.1 |
| whole genome shotgun sequence | |
| Clostridium diolis strain WST Scaffold15_1, whole genome shotgun | WP_011967971.1 |
| sequence | |
| Clostridium beijerinckii strain ATCC 39058 CBEI_contig000004, | WP_011967971.1 |
| whole genome shotgun sequence | |
| Clostridium diolis strain NJP7 scaffold2, whole genome shotgun | WP_011967971.1 |
| sequence | |
| Clostridium beijerinckii strain NCTC13035, whole genome shotgun | WP_011967971.1 |
| sequence | |
| Clostridium beijerinckii strain BAS/B3/1/124, complete genome | WP_011967971.1 |
| Clostridium sp. MF28, genome | WP_011967971.1 |
| Clostridium beijerinckii NRRL B-598 chromosome, complete | WP_011967971.1 |
| genome | |
| Clostridium beijerinckii strain NCIMB 14988, complete genome | WP_011967971.1 |
| Clostridium beijerinckii strain NRRL B-593 CLOBI_contig000172, | WP_011967971.1 |
| whole genome shotgun sequence | |
| Clostridium beijerinckii strain NRRL B-528 | WP_011967971.1 |
| CLBEIC contig000055, whole genome shotgun sequence | |
| Clostridium beijerinckii isolate C. beijerinckii DSM 6423 genome | WP_011967971.1 |
| assembly, chromosome: I | |
| Clostridium beijerinckii strain NRRL B-596 CLOBE_contig000006, | WP_077854106.1 |
| whole genome shotgun sequence | |
| Clostridium sp. BL-8 CLOBL_contig000019, whole genome | WP_077858646.1 |
| shotgun sequence | |
| Ruminococcus sp. HUN007 | WP_044974741.1 |
| CC97DRAFT_scf7180000000020_quiver.2_C, whole genome | |
| shotgun sequence | |
| Siculibacillus lacustris strain SA-279 scaffold_6, whole genome | WP_131307352.1 |
| shotgun sequence | |
| Pelosinus sp. UFO1, complete genome | WP_038671808.1 |
| Pectinatus cerevisiiphilus strain DSM 20467 Ga0244680_115, | WP_132550791.1 |
| whole genome shotgun sequence | |
| Clostridium tyrobutyricum isolate MGYG-HGUT-00125, whole | WP_017751332.1 |
| genome shotgun sequence | |
| Dendrosporobacter quercicolus strain DSM 1736, whole genome | WP_092071615.1 |
| shotgun sequence | |
| Rhodopseudomonas palustris strain YSC3 chromosome, complete | WP_107355446.1 |
| genome | |
| Sporomusaceae bacterium strain FL31 scf_SPFL3102_001, whole | WP_127032830.1 |
| genome shotgun sequence | |
| Sporomusaceae bacterium FL31 scf_SPFL3101_011, whole genome | WP_127032830.1 |
| shotgun sequence | |
| Ruminiclostridium hungatei strain DSM 14427 | WP_080066050.1 |
| CLHUN contig000028, whole genome shotgun sequence | |
| Propionispora vibrioides strain DSM 13305, whole genome shotgun | WP_091748268.1 |
| sequence | |
| Paenibacillus durus ATCC 35681, complete genome | WP_025697960.1 |
| Rhodopseudomonas palustris strain PS3 chromosome, complete | WP_107344277.1 |
| genome | |
| Sporomusa sp. KB1 SalpaDRAFT_Scaffold1.2, whole genome | WP_145096946.1 |
| shotgun sequence | |
| Propionispora sp. 2/2-37, whole genome shotgun sequence | WP_054261180.1 |
| Clostridium pasteurianum strain W5 contig00122, whole genome | WP_003446488.1 |
| shotgun sequence | |
| Clostridium sp. BNL1100, complete genome | WP_014313379.1 |
| Paenibacillus stellifer strain DSM 14472, complete genome | WP_038694277.1 |
| Ruminiclostridium josui JCM 17888 | WP_024834618.1 |
| K412DRAFT_scf7180000000007_quiver.2_C, whole genome | |
| shotgun sequence | |
| Rhodopseudomonas palustris strain ELI 1980 Contig20, whole | WP_011157906.1 |
| genome shotgun sequence | |
| Rhodopseudomonas palustris CGA009 complete genome | WP_011157906.1 |
| Rhodopseudomonas palustris TIE-1, complete genome | WP_012495829.1 |
| Clostridium chromiireducens strain C1 Scaffold1, whole genome | WP_079440385.1 |
| shotgun sequence | |
| Rhodomicrobium sp. JA980 | WP_127079012.1 |
| NODE_13_length_1721687_cov_26.857853, whole genome | |
| shotgun sequence | |
| Clostridium tyrobutyricum strain Cirm BIA 2237 chromosome | WP_017895276.1 |
| Paenibacillus sabinae T27, complete genome | WP_025334792.1 |
| Clostridium pasteurianum DSM 525 = ATCC 6013 ctg1, whole | WP_003446488.1 |
| genome shotgun sequence | |
| Clostridium ljungdahlii DSM 13528, complete genome | WP_013237172.1 |
| Clostridium autoethanogenum DSM 10061, complete genome | WP_013237172.1 |
| Clostridium autoethanogenum DSM 10061, complete genome | WP_013237172.1 |
| Clostridium pasteurianum strain M150B, complete genome | WP_003446488.1 |
| Clostridium pasteurianum DSM 525 = ATCC 6013, complete | WP_003446488.1 |
| genome | |
| Clostridium pasteurianum DSM 525 = ATCC 6013, complete | WP_003446488.1 |
| genome | |
| Clostridium pasteurianum DSM 525 = ATCC 6013, complete | WP_003446488.1 |
| genome | |
| Clostridium pasteurianum BC1, complete genome | WP_015617157.1 |
| Clostridium sp. DL-VIII chromosome, whole genome shotgun | WP_009167878.1 |
| sequence | |

In some embodiments, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 4 (MarH).

SEQ ID NO: 4
MAKSPKQIAIYGKGGIGKSTTTSNISAALAEAGYKVMQFG
CDPKSDSTNTLRGGDYIPSVLDLLRENARVDAHEAIFQGF
GGIYCVEAGGPAPGVGCAGRGIITAVELLKQQNVFEELDL
DYVIFDVLGDVVCGGFAVPIREGIAEHVFTVSSSDFMAIY
AANNLFKGIQKYSNAGGALLGGVIANSINTDFHRDIIDDF
VARTQTQVVQYVPRSLTVTQAELQGRTTIEAAPESAQAEI
YRTLARSIADHTDSKVPTPLNAQELRDWSASWANQLIEIE
RASQPIPALAS

In some embodiments, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 4. In some embodiments, the one or more genes of a methylthio-alkane reductase complex comprise a gene encoding a protein of SEQ ID NO: 4. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments, the one or more genes of a methylthio-alkane reductase complex comprise marD. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 5 (marD).

SEQ ID NO: 5
ATGCCCATCAATCTCAAGACATCGGTGGTCGAGAGCCGCG
AACAGCGGCTGGGCACCATCATCGCCTGGGACGGCAAGGC
CTCTGACCTGTCCAAGGAATCGGCCTATGCGCGCAGCGAG
GGCTGCGGCAGCGCCTGCGGCGCCAAGGCCCGCCGGGTCT
GCGAGATGCGCAGCCCGTTCAGCCAGGGCTCGGTCTGTAG
CGAACAGATGGTCGAATGCCAAGCCGGCAACGTGCGCGGC
GCCGTGCTGGTCCAGCATTCGCCGATCGGCTGCGGCGCCG
GTCAGGTGATCTATAATTCGATCTTCCGCAATGGTCTGGC
GATCCGCGGCCTGCCGGTGGAGAACCTCCATCTGATCAGC
ACCAACCTGCGCGAACGCGACATGGTCTATGGCGGGCTCG
ACAAGCTCGAACGCACCATCCGCGACGCCTGGGAGCGCCA
TCACCCCCAGGCCATTTTCATCGCCACCTCCTGCCCGACG
GCGATCATTGGCGACGACATCGAAAGCGTCGCTTCGCAGC
TTGAAGCCGAGTTCGGCATACCGGTCATACCGCTGCACTG
CGAGGGCTTCAAATCCAAGCATTGGAGCACCGGCTTCGAC
GCCACCCAGCACGGCATCTTGCGCCAGATCGTCCGCAAAA
ATCCCGAGCGCAAGCAGGAAGACCTGGTCAACGTCATCAA
TCTGTGGGGATCGGATGTCTTTGGCCCGATGCTCGGCGAA
TTGGGTTTGCGGGTGAACTACGTCGTCGATCTCGCCACCG
TCGAGGATCTGGCCCAGATGTCGGAGGCGGCGGCAACCGT
CGGCTTCTGCTACACGCTGTCGACCTATATGGCCGCCGCC
CTGGAACAGGAATTCGGCGTTCCCGAGGTCAAGGCGCCCA
TGCCCTATGGCTTCGCCGGCACCGACGCCTGGCTGCGCGA
GATCGCCCGCGTCACCCACCGCGAGGAGCAGGCCGAGGCC
TATATCGCCCGCGAGCACGCCCGGGTGAAGCCACAGCTTG
AGGCCCTGCGCGAGAAGCTCAAGGGCATCAAGGGCTTCGT
CTCCACCGGCTCGGCCTATGCCCATGGCATGATCCAGGTG
CTGCGCGAACTGGGCGTCACCGTCGACGGCTCGTTGGTCT
TCCACCACGATCCGGTCTACGACAGCCAGGATCCGCGTCA
GGATTCCCTTGCCCATCTGGTCGACAACTATGGCGACGTC
GGCCATTTCAGCGTCGGCAATCGCCAGCAGTTCCAGTTCT
ACGGCCTGCTTCAGCGGGTGAAGCCCGATTTCATCATCAT
CCGCCACAACGGGTTGGCGCCGCTGGCCTCGCGCCTGGGC
ATCCCGGCCATTCCGCTGGGCGATGAACATATCGCCGTGG
GCTATCAGGGCATCTTGAACCTGGGTGAATCCATCCTCGA
TGTGCTGGCCCACCGCAAGTTCCACGAAGACATCGCCGCC
CATGTCCGCCTGCCCTATCGCCAGGACTGGCTGGCCCGCG
ATCCCTTCGATCTGGCCCGGCAAAGCGCCGGCCAGCCGCG
CCGTCCCGCAGAGTGA

In some embodiments, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 5. In some embodiments, the one or more genes of a methylthio-alkane reductase complex comprise a nucleic acid sequence of SEQ ID NO: 5.

In some embodiments, the one or more genes of a methylthio-alkane reductase complex comprise one or more marD genes associated with an accession number found in Table 2 below:

TABLE 2
Representative MarD Genes

| Whole Genome Sequence of Origin | Accession Number |
|---|---|
| Pararhodospirillum oryzae strain NBRC 107573 sequence093, | WP_147164650.1 |
| whole genome shotgun sequence | |
| Rhodospirillum photometricum DSM 122 draft genome sequence | WP_041796109.1 |
| Rhodospirillum rubrum ATCC 11170 chromosome, complete | YP 425885.1 |
| genome | |
| Rhodospirillum rubrum F11, complete genome | WP_011388552.1 |
| Rhodomicrobium udaipurense JA643 contig00206, whole genome | WP_037242222.1 |
| shotgun sequence | |
| Rhodoblastus sphagnicola strain DSM 16996 scaffold0018, whole | WP_104506082.1 |
| genome shotgun sequence | |
| Rhodoblastus acidophilus strain DSM 137 | WP_088522710.1 |
| NODE_116_length_9951_cov_47.3758, whole genome shotgun | |
| sequence | |
| Rhodoblastus acidophilus strain DSM 137, whole genome shotgun | WP_088522710.1 |
| sequence | |
| Rhodoblastus acidophilus strain DSM 137 scaffold0022, whole | WP_088522710.1 |
| genome shotgun sequence | |
| Rhodomicrobium sp. JA980 | WP_127076530.1 |
| NODE_3_length_364448_cov_26.852217, whole genome shotgun | |
| sequence | |
| Pleomorphomonas carboxyditropha strain SVCO-16 | WP_100079640.1 |
| NODE_2_length_581917_cov_22.4871, whole genome shotgun | |
| sequence | |
| Rhodomicrobium vannielii ATCC 17100, complete genome | WP_013421121.1 |
| Thermoanaerobacterium thermosaccharolyticum M0795, complete | WP_015311769.1 |
| genome | |
| Bacteroidales bacterium Barb6XT Barb6XT_contig_167, whole | WP_066182118.1 |
| genome shotgun sequence | |
| Prevotella bryantii strain TC1-1 contig9, whole genome shotgun | WP_006281724.1 |
| sequence | |
| Selenomonas ruminantium strain WCT3, whole genome shotgun | WP_074513506.1 |
| sequence | |
| Selenomonas sp. ND2010 T504DRAFT_scaffold00003.3_C, whole | WP_033169627.1 |
| genome shotgun sequence | |
| Phaeospirillum fulvum strain DSM 13234, whole genome shotgun | WP_074764657.1 |
| sequence | |
| Clostridium coskatii strain PTA-10522 CLCOS contig000056, | WP_063601657.1 |
| whole genome shotgun sequence | |
| Clostridium coskatii strain PS02 scaffold19_1_86601, whole | WP_063601657.1 |
| genome shotgun sequence | |
| Clostridium autoethanogenum strain H21-9 Contig_058, whole | WP_122059870.1 |
| genome shotgun sequence | |
| Clostridium ljungdahlii DSM 13528 strain PETC | WP_013239000.1 |
| scaffold3 200123 404054, whole genome shotgun sequence | |
| Clostridium drakei strain SLI contig_79, whole genome shotgun | WP_032079661.1 |
| sequence | |
| Clostridium drakei strain SLI chromosome, complete genome | WP_032079661.1 |
| Clostridium scatologenes strain ATCC 25775, complete genome | WP_029160438.1 |
| Fibrobacter sp. UWT2, whole genome shotgun sequence | WP_072801409.1 |
| Fibrobacter sp. UWB8, whole genome shotgun sequence | WP_073056571.1 |
| Fibrobacter sp. UWB6 Ga0136278_108, whole genome shotgun | WP_073056571.1 |
| sequence | |
| Fibrobacter sp. UWB15, whole genome shotgun sequence | WP_073056571.1 |
| Fibrobacter sp. UWB5 NODE_1, whole genome shotgun sequence | WP_072801409.1 |
| Fibrobacter sp. UWBI NODE_4, whole genome shotgun sequence | WP_088657010.1 |
| Fibrobacter sp. UWOVI, whole genome shotgun sequence | WP_073321572.1 |
| Fibrobacter sp. UWH4, whole genome shotgun sequence | WP_072977616.1 |
| Selenomonas bovis 8-14-1 T485DRAFT_scaffold00002.2_C, whole | WP_031584321.1 |
| genome shotgun sequence | |
| Rhodopseudomonas palustris strain 2.1.37 scaffold_36, whole | WP_011158186.1 |
| genome shotgun sequence | |
| Rhodopseudomonas palustris strain DSM 126 scaffold0001, whole | WP_011158186.1 |
| genome shotgun sequence | |
| Rhodopseudomonas palustris strain RI | WP_119018515.1 |
| NODE_28_length_158663_cov_40.885563, whole genome shotgun | |
| sequence | |
| Rhodopseudomonas sp. AAP120 AAP120_Contigs_11, whole | WP_054160732.1 |
| genome shotgun sequence | |
| Fibrobacter succinogenes subsp. succinogenes S85, complete | WP_015731913.1 |
| genome | |
| Fibrobacter succinogenes subsp. succinogenes S85, complete | WP_015731913.1 |
| genome | |
| Blastochloris viridis genome assembly Blastochloris viridis genome, | WP_055038750.1 |
| chromosome : I | |
| Blastochloris viridis strain ATCC 19567, complete genome | WP_055038750.1 |
| Blastochloris viridis DNA, complete genome, strain: DSM 133 | WP_055038750.1 |
| Clostridium autoethanogenum DSM 10061 seq4, whole genome | WP_023161825.1 |
| shotgun sequence | |
| Clostridium autoethanogenum strain JA1-1 | WP_023161825.1 |
| scaffold2 136726 570037, whole genome shotgun sequence | |
| Ruminococcaceae bacterium HV4-5-BSC, whole genome shotgun | WP_114174822.1 |
| sequence | |
| Clostridium bornimense replicon M2/40_rep1, complete genome, | WP_044035925.1 |
| type strain M2/40T | |
| Clostridium ljungdahlii strain ERI-2 scaffold7, whole genome | WP_063557082.1 |
| shotgun sequence | |
| Clostridium chromiireducens strain DSM 23318 | WP_079439998.1 |
| CLCHR contig000029, whole genome shotgun sequence | |
| Rhodopseudomonas palustris strain RI | WP_119017316.1 |
| NODE_7_length_89266_cov_41.693230, whole genome shotgun | |
| sequence | |
| Rhodopseudomonas palustris strain DSM 126 scaffold0020, whole | WP_011157901.1 |
| genome shotgun sequence | |
| Rhodopseudomonas palustris strain 2.1.37 scaffold_3, whole | WP_011157901.1 |
| genome shotgun sequence | |
| Pleomorphomonas carboxyditropha strain SVCO-16 | WP_100081803.1 |
| NODE_13_length_137005_cov_21.4606, whole genome shotgun | |
| sequence | |
| Pleomorphomonas sp. CF100 Ga0189743_114, whole genome | WP_134185343.1 |
| shotgun sequence | |
| Pleomorphomonas koreensis DSM 23070 | WP_026783522.1 |
| HS12DRAFT_scaffold00010.10_C, whole genome shotgun | |
| sequence | |
| Roseiarcus fermentans strain DSM 24875 Ga0244512_102, whole | WP_113887560.1 |
| genome shotgun sequence | |
| Ruminococcus flavefaciens strain XPD3002, whole genome shotgun | WP_075423703.1 |
| sequence | |
| Clostridium beijerinckii strain NR.RL B-591 CLBKI_contig000007, | WP_011967979.1 |
| whole genome shotgun sequence | |
| Clostridium beijerinckii strain 4J9 CLOSB_contig000013, whole | WP_011967979.1 |
| genome shotgun sequence | |
| Clostridium beijerinckii ATCC 35702, complete genome | WP_011967979.1 |
| Clostridium beijerinckii NCIMB 8052, complete genome | WP_011967979.1 |
| Clostridium beijerinckii G117 Scaffold22, whole genome shotgun | WP_017212478.1 |
| sequence | |
| Clostridium beijerinckii strain WB | WP_017212478.1 |
| Clostridium_beijerinckii_WB_contig15, whole genome shotgun | |
| sequence | |
| Clostridium beijerinckii strain DSM 791 CLBEI_contig000075, | WP_017212478.1 |
| whole genome shotgun sequence | |
| Clostridium beijerinckii strain NBRC 109359 sequence070, whole | WP_017212478.1 |
| genome shotgun sequence | |
| Clostridium beijerinckii strain BAS/B2 CLBEJ_contig000034, | WP_077304248.1 |
| whole genome shotgun sequence | |
| Clostridium beijerinckii strain NCP 260 CLOBJ_contig000033, | WP_077304248.1 |
| whole genome shotgun sequence | |
| Clostridium diolis strain WST Scaffold15_1, whole genome shotgun | WP_017212478.1 |
| sequence | |
| Clostridium beijerinckii strain ATCC 39058 CBEIJ_contig000004, | WP_017212478.1 |
| whole genome shotgun sequence | |
| Clostridium diolis strain NJP7 scaffold2, whole genome shotgun | WP_087701226.1 |
| sequence | |
| Clostridium beijerinckii strain NCTC13035, whole genome shotgun | WP_017212478.1 |
| sequence | |
| Clostridium beijerinckii strain BAS/B3/I/124, complete genome | WP_077304248.1 |
| Clostridium sp. MF28, genome | WP_017212478.1 |
| Clostridium beijerinckii NRRL B-598 chromosome, complete | WP_023973644.1 |
| genome | |
| Clostridium beijerinckii strain NCIMB 14988, complete genome | WP_041894108.1 |
| Clostridium beijerinckii strain NRRL B-593 CLOBI_contig000172, | WP_077843816.1 |
| whole genome shotgun sequence | |
| Clostridium beijerinckii strain NRRL B-528 | WP_077843816.1 |
| CLBEIC contig000055, whole genome shotgun sequence | |
| Clostridium beijerinckii isolate C. beijerinckii DSM 6423 genome | WP_077843816.1 |
| assembly, chromosome: I | |
| Clostridium beijerinckii strain NRRL B-596 CLOBE_contig000006, | WP_077854103.1 |
| whole genome shotgun sequence | |
| Clostridium sp. BL-8 CLOBL_contig000019, whole genome | WP_077858635.1 |
| shotgun sequence | |
| Ruminococcus sp. HUN007 | WP_044974747.1 |
| CC97DRAFT_scf7180000000020_quiver.2_C, whole genome | |
| shotgun sequence | |
| Siculibacillus lacustris strain SA-279 scaffold_6, whole genome | WP_131307356.1 |
| shotgun sequence | |
| Pelosinus sp. UFO1, complete genome | WP_038671836.1 |
| Pectinatus cerevisiiphilus strain DSM 20467 Ga0244680_115, | WP_132550766.1 |
| whole genome shotgun sequence | |
| Clostridium tyrobutyricum isolate MGYG-HGUT-00125, whole | WP_017894496.1 |
| genome shotgun sequence | |
| Dendrosporobacter quercicolus strain DSM 1736, whole genome | WP_092071670.1 |
| shotgun sequence | |
| Rhodopseudomonas palustris strain YSC3 chromosome, complete | WP_107355473.1 |
| genome | |
| Sporomusaceae bacterium strain FL31 scf_SPFL3102_001, whole | WP_127032899.1 |
| genome shotgun sequence | |
| Sporomusaceae bacterium FL31 scf_SPFL3101_011, whole genome | WP_127032899.1 |
| shotgun sequence | |
| Ruminiclostridium hungatei strain DSM 14427 | WP_080066007.1 |
| CLHUN_contig000028, whole genome shotgun sequence | |
| Propionispora vibrioides strain DSM 13305, whole genome shotgun | WP_091748362.1 |
| sequence | |
| Paenibacillus durus ATCC 35681, complete genome | WP_025700551.1 |
| Rhodopseudomonas palustris strain PS3 chromosome, complete | WP_107344317.1 |
| genome | |
| Sporomusa sp. KB1 SalpaDRAFT_Scaffold1.2, whole genome | WP_145096800.1 |
| shotgun sequence | |
| Propionispora sp. 2/2-37, whole genome shotgun sequence | WP_054261135.1 |
| Clostridium pasteurianum strain W5 contig00122, whole genome | WP_003444630.1 |
| shotgun sequence | |
| Clostridium sp. BNL 1100, complete genome | WP_014313542.1 |
| Paenibacillus stellifer strain DSM 14472, complete genome | WP_038694489.1 |
| Ruminiclostridium josui JCM 17888 | WP_024834403.1 |
| K412DRAFT_scf7180000000007_quiver.2_C, whole genome | |
| shotgun sequence | |
| Rhodopseudomonas palustris strain ELI 1980 Contig20, whole | WP_011158186.1 |
| genome shotgun sequence | |
| Rhodopseudomonas palustris CGA009 complete genome | WP_011158186.1 |
| Rhodopseudomonas palustris TIE-1, complete genome | WP_012496075.1 |
| Clostridium chromiireducens strain C1 Scaffold1, whole genome | WP_119365464.1 |
| shotgun sequence | |
| Rhodomicrobium sp. JA980 | WP_127077350.1 |
| NODE_13_length_1721687_cov_26.857853, whole genome | |
| shotgun sequence | |
| Clostridium tyrobutyricum strain Cirm BIA 2237 chromosome | WP_017894496.1 |
| Paenibacillus sabinae T27, complete genome | WP_025336405.1 |
| Clostridium pasteurianum DSM 525 = ATCC 6013 ctg1, whole | WP_003444630.1 |
| genome shotgun sequence | |
| Clostridium ljungdahlii DSM 13528, complete genome | WP_013239000.1 |
| Clostridium autoethanogenum DSM 10061, complete genome | WP_023161825.1 |
| Clostridium autoethanogenum DSM 10061, complete genome | WP_023161825.1 |
| Clostridium pasteurianum strain M150B, complete genome | WP_003444630.1 |
| Clostridium pasteurianum DSM 525 = ATCC 6013, complete | WP_003444630.1 |
| genome | |
| Clostridium pasteurianum DSM 525 = ATCC 6013, complete | WP_003444630.1 |
| genome | |
| Clostridium pasteurianum DSM 525 = ATCC 6013, complete | WP_003444630.1 |
| genome | |
| Clostridium pasteurianum BC1, complete genome | WP_015614355.1 |
| Clostridium sp. DL-VIII chromosome, whole genome shotgun | WP_009172467.1 |
| sequence | |

In some embodiments, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 6 (MarD).

SEQ ID NO: 6
MPINLKTSVVESREQRLGTIIAWDGKASDLSKESAYARSE
GCGSACGAKARRVCEMRSPFSQGSVCSEQMVECQAGNVRG
AVLVQHSPIGCGAGQVIYNSIFRNGLAIRGLPVENLHLIS
TNLRERDMVYGGLDKLERTIRDAWERHHPQAIFIATSCPT
AIIGDDIESVASQLEAEFGIPVIPLHCEGFKSKHWSTGFD
ATQHGILRQIVRKNPERKQEDLVNVINLWGSDVFGPMLGE
LGLRVNYVVDLATVEDLAQMSEAAATVGFCYTLSTYMAAA
LEQEFGVPEVKAPMPYGFAGTDAWLREIARVTHREEQAEA
YIAREHARVKPQLEALREKLKGIKGFVSTGSAYAHGMIQV
LRELGVTVDGSLVFHHDPVYDSQDPRQDSLAHLVDNYGDV
GHFSVGNRQQFQFYGLLQRVKPDFIIIRHNGLAPLASRLG
IPAIPLGDEHIAVGYQGILNLGESILDVLAHRKFHEDIAA
HVRLPYRQDWLARDPFDLARQSAGQPRRP

In some embodiments, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 6. In some embodiments, the one or more genes of a methylthio-alkane reductase complex comprise a gene encoding a protein of SEQ ID NO: 6. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments, the one or more genes of a methyltbio-alkane reductase complex comprise marK. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 7 (marK).

SEQ ID NO: 7
ATGCCCGATGCAGAGTCCCGTTCCCAGGTCACGGCGAAGG
CCGCGCCACCACCCGCCCCCAAGACCAATTCGATCGAACA
GGTGCGCTATATCTGTTCGATCGGCGCCATGCACAGCGCC
TCGGCTATCCCACGGGTGATCCCGATCACCCATTGCGGCC
CGGGCTGCGCCGACAAGCAGTTCATGAACGTCGCCTTCTA
TAATGGCTTCCAGGGCGGCGGCTATGGCGGCGGAGCGGTG
GTGCCGAGCACCAACGCCACCGAGCGCGAGGTGGTCTTCG
GCGGCGCCGAGCGCCTGGACGAATTGATCGGCGCCTCGCT
GCAGGTGCTTGACGCCGACCTGTTCGTGGTGCTGACCGGC
TGTATTCCCGATCTGGTCGGCGATGACATCGGCTCGGTGG
TCGGCCCCTATCAGAAGCGCGGCGTGCCGATCGTCTATGC
CGAGACTGGCGGCTTTCGCGGCAATAACTTCACCGGCCAC
GAACTGGTGACCAAGGCGATCATCGACCAGTTCGTTGGCG
ATTACGATGCGGAGCGCGACGGGGCCCGCGAGCCCCATAC
GGTCAATGTCTGGTCACTGCTGCCCTACCACAACACCTTC
TGGCGCGGTGATTTGACCGAGATCAAGCGGCTGCTCGAAG
GCATCGGCCTTAAGGTCAATATCCTGTTCGGCCCGCAATC
GGCCGGGGTGGCGGAATGGAAGGCCATCCCGCGCGCCGGC
TTTAATCTGGTGCTCTCGCCCTGGCTGGGGCTGGACACGG
CGCGCCATTTGGACCGCAAATACGGCCAGCCGACCCTGCA
TCGACCGATCATCCCGATCGGCGCCAAGGAAACCGGCGCC
TTCCTGCGCGAGGTGGCGGCTTTCGCCGGCCTCGACAGCG
CGGTGGTCGAGGCCTTCATCACCGCCGAAGAAGCCGTTTA
TTACCGCTATCTGGAGGACTTCACCGATTTCTACGCGGAG
TACTGGTGGGGTCTGCCGGCCAAATTCGCCGTCATCGGCG
ACAGCGCCTATAATCTGGCCTTGACCAAATTCCTGGTAAA
CCAGTTGGGCCTGATACCGGGGCTGCAGATCATCACCGAC
AATCCGCCCGAGGAGGTGCGCGAGGATATCCGCGCCCATT
ACCACGCGATCGCCGATGACGTGGCCACCGATGTCTCTTT
TGAAGAAGACAGCTACACCATCCACCAAAAGATCCGCGCC
ACCGATTTCGGCCACAAGGCGCCGATCCTGTTTGGCACCA
CCTGGGAACGCGACCTTGCCAAGGAATTGAAGGGGGCGAT
CGTCGAGGTCGGCTTCCCGGCATCCTATGAAGTCGTGCTG
TCGCGCAGCTATCTTGGCTACCGGGGCGCCCTGACTTTGC
TGGAAAAAATCTACACAACCACCGTCAGCGCAAGCGCTTG
A

In some embodiments, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 7. In some embodiments, the one or more genes of a methylthio-alkane reductase complex comprise a nucleic acid sequence of SEQ ID NO: 7.

In some embodiments, the one or more genes of a methylthio-alkane reductase complex comprise one or more marK genes associated with an accession number found in Table 3 below:

TABLE 3
Representative Mark Genes

| Whole Genome Sequence of Origin | Accession Number |
|---|---|
| Pararhodospirillum oryzae strain NBRC 107573 sequence093, | WP_147164649.1 |
| whole genome shotgun sequence | |
| Rhodospirillum photometricum DSM 122 draft genome sequence | WP_014416390.1 |
| Rhodospirillum rubrum ATCC 11170 chromosome, complete | YP_425884.1 |
| genome | |
| Rhodospirillum rubrum F11, complete genome | WP_011388551.1 |
| Rhodomicrobium udaipurense JA643 contig00206, whole genome | pseudo/ |
| shotgun sequence | frameshift |
| Phaeospirillum fulvum MGU-K5 contig00054, whole genome | WP_021132882.1 |
| shotgun sequence | |
| Rhodoblastus sphagnicola strain DSM 16996 scaffold0018, whole | WP_104506081.1 |
| genome shotgun sequence | |
| Rhodoblastus acidophilus strain DSM 137 | WP_088522709.1 |
| NODE_116_length_9951_cov_47.3758, whole genome shotgun | |
| sequence | |
| Rhodoblastus acidophilus strain DSM 137, whole genome shotgun | WP_088522709.1 |
| sequence | |
| Rhodoblastus acidophilus strain DSM 137 scaffold0022, whole | WP_141098569.1 |
| genome shotgun sequence | |
| Rhodomicrobium sp. JA980 | WP_127076532.1 |
| NODE_3_length_364448_cov_26.852217, whole genome shotgun | |
| sequence | |
| Pleomorphomonas carboxyditropha strain SVCO-16 | WP_100079639.1 |
| NODE_2_length_581917_cov_22.4871, whole genome shotgun | |
| sequence | |
| Rhodomicrobium vannielii ATCC 17100, complete genome | WP_013421122.1 |
| Thermoanaerobacterium thermosaccharolyticum M0795, complete | WP_015311768.1 |
| genome | |
| Bacteroidales bacterium Barb6XT Barb6XT_contig_167, whole | WP_066182121.1 |
| genome shotgun sequence | |
| Prevotella bryantii strain TC1-1 contig9, whole genome shotgun | WP_094447989.1 |
| sequence | |
| Selenomonas ruminantium strain WCT3, whole genome shotgun | WP_074513505.1 |
| sequence | |
| Selenomonas sp. ND2010 T504DRAFT scaffold00003.3 C, whole | WP_033169626.1 |
| genome shotgun sequence | |
| Phaeospirillum fulvum strain DSM 13234, whole genome shotgun | WP_074764659.1 |
| sequence | |
| Clostridium coskatii strain PTA-10522 CLCOS_contig000056, | WP_063601656.1 |
| whole genome shotgun sequence | |
| Clostridium coskatii strain PS02 scaffold19 1 86601, whole | WP_063601656.1 |
| genome shotgun sequence | |
| Clostridium autoethanogenum strain H21-9 Contig_058, whole | WP_122059871.1 |
| genome shotgun sequence | |
| Clostridium ljungdahlii DSM 13528 strain PETC | WP_081442103.1 |
| scaffold3 200123 404054, whole genome shotgun sequence | |
| Clostridium drakei strain SLI contig_79, whole genome shotgun | WP_032079662.1 |
| sequence | |
| Clostridium drakei strain SLI chromosome, complete genome | WP_108849503.1 |
| Clostridium scatologenes strain ATCC 25775, complete genome | WP_029160439.1 |
| Fibrobacter sp. UWT2, whole genome shotgun sequence | WP_072801410.1 |
| Fibrobacter sp. UWB8, whole genome shotgun sequence | WP_073056570.1 |
| Fibrobacter sp. UWB6 Ga0136278_108, whole genome shotgun | WP_073056570.1 |
| sequence | |
| Fibrobacter sp. UWB15, whole genome shotgun sequence | WP_073056570.1 |
| Fibrobacter sp. UWB5 NODE_1, whole genome shotgun sequence | WP_088626792.1 |
| Fibrobacter sp. UWBI NODE_4, whole genome shotgun sequence | WP_088657009.1 |
| Fibrobacter sp. UWOV1, whole genome shotgun sequence | WP_073321575.1 |
| Fibrobacter sp. UWH4, whole genome shotgun sequence | WP_072977614.1 |
| Selenomonas bovis 8-14-1 T485DRAFT scaffold00002.2 C, whole | WP_031584319.1 |
| genome shotgun sequence | |
| Rhodopseudomonas palustris strain 2.1.37 scaffold 36, whole | WP_011158187.1 |
| genome shotgun sequence | |
| Rhodopseudomonas palustris strain DSM 126 scaffold0001, whole | WP_011158187.1 |
| genome shotgun sequence | |
| Rhodopseudomonas palustris strain R1 | WP_119018514.1 |
| NODE_28_length_158663_cov 40.885563, whole genome shotgun | |
| sequence | |
| Rhodopseudomonas sp. AAP120 AAP120_Contigs_11, whole | WP_054160733.1 |
| genome shotgun sequence | |
| Fibrobacter succinogenes subsp. succinogenes S85, complete | WP_014545821.1 |
| genome | |
| Fibrobacter succinogenes subsp. succinogenes S85, complete | WP_014545821.1 |
| genome | |
| Blastochloris viridis genome assembly Blastochloris viridis genome, | WP_055037159.1 |
| chromosome : I | |
| Blastochloris viridis strain ATCC 19567, complete genome | WP_055037159.1 |
| Blastochloris viridis DNA, complete genome, strain: DSM 133 | WP_055037159.1 |
| Clostridium autoethanogenum DSM 10061 seq4, whole genome | WP_023161824.1 |
| shotgun sequence | |
| Clostridium autoethanogenum strain JA1-1 | WP_023161824.1 |
| scaffold2 136726 570037, whole genome shotgun sequence | |
| Ruminococcaceae bacterium HV4-5-B5C, whole genome shotgun | WP_114174613.1 |
| sequence | |
| Clostridium bornimense replicon M2/40_rep1, complete genome, | WP_044035926.1 |
| type strain M2/40T | |
| Clostridium ljungdahlii strain ERI-2 scaffold7, whole genome | WP_063557118.1 |
| shotgun sequence | |
| Clostridium chromiireducens strain DSM 23318 | WP_079439997.1 |
| CLCHR contig000029, whole genome shotgun sequence | |
| Rhodopseudomonas palustris strain R1 | WP_119017317.1 |
| NODE_7_length_89266_cov_41.693230, whole genome shotgun | |
| sequence | |
| Rhodopseudomonas palustris strain DSM 126 scaffold0020, whole | WP_011157900.1 |
| genome shotgun sequence | |
| Rhodopseudomonas palustris strain 2.1.37 scaffold 3, whole | WP_011157900.1 |
| genome shotgun sequence | |
| Pleomorphomonas carboxyditropha strain SVCO-16 | WP_100081802.1 |
| NODE_13_length_137005_cov_21.4606, whole genome shotgun | |
| sequence | |
| Pleomorphomonas sp. CF100 Ga0189743 114, whole genome | WP_134185341.1 |
| shotgun sequence | |
| Pleomorphomonas koreensis DSM 23070 | WP_036791276.1 |
| H512DRAFT scaffold00010.10_C, whole genome shotgun | |
| sequence | |
| Roseiarcus fermentans strain DSM 24875 Ga0244512_102, whole | WP_113887559.1 |
| genome shotgun sequence | |
| Ruminococcus flavefaciens strain XPD3002, whole genome shotgun | WP_075423704.1 |
| sequence | |
| Clostridium beijerinckii HUN142 T483DRAFT scaffold00009.9 C, | WP_026886168.1 |
| whole genome shotgun sequence | |
| Clostridium beijerinckii strain NRRL B-591 CLBKI_contig000007, | WP_011967980.1 |
| whole genome shotgun sequence | |
| Clostridium beijerinckii strain 4J9 CLOSB_contig000013, whole | WP_011967980.1 |
| genome shotgun sequence | |
| Clostridium beijerinckii ATCC 35702, complete genome | WP_011967980.1 |
| Clostridium beijerinckii NCIMB 8052, complete genome | WP_011967980.1 |
| Clostridium beijerinckii G117 Scaffold22, whole genome shotgun | WP_017212477.1 |
| sequence | |
| Clostridium beijerinckii strain WB | WP_017212477.1 |
| Clostridium_beijerinckii_WB_contig15, whole genome shotgun | |
| sequence | |
| Clostridium beijerinckii strain DSM 791 CLBEI_contig000075, | WP_039773292.1 |
| whole genome shotgun sequence | |
| Clostridium beijerinckii strain NBRC 109359 sequence070, whole | WP_039773292.1 |
| genome shotgun sequence | |
| Clostridium beijerinckii strain BAS/B2 CLBEJ_contig000034, | WP_077304251.1 |
| whole genome shotgun sequence | |
| Clostridium beijerinckii strain NCP 260 CLOBJ_contig000033, | WP_077304251.1 |
| whole genome shotgun sequence | |
| Clostridium diolis strain WST Scaffold15_1, whole genome shotgun | WP_039773292.1 |
| sequence | |
| Clostridium beijerinckii strain ATCC 39058 CBEIJ_contig000004, | WP_039773292.1 |
| whole genome shotgun sequence | |
| Clostridium diolis strain NJP7 scaffold2, whole genome shotgun | WP_087701225.1 |
| sequence | |
| Clostridium beijerinckii strain NCTC13035, whole genome shotgun | WP_039773292.1 |
| sequence | |
| Clostridium beijerinckii strain BAS/B3/1/124, complete genome | WP_077304251.1 |
| Clostridium sp. MF28, genome | WP_039773292.1 |
| Clostridium beijerinckii NRRL B-598 chromosome, complete | WP_023973643.1 |
| genome | |
| Clostridium beijerinckii strain NCIMB 14988, complete genome | WP_041894111.1 |
| Clostridium beijerinckii strain NRRL B-593 CLOBI_contig000172, | WP_077843817.1 |
| whole genome shotgun sequence | |
| Clostridium beijerinckii strain NRRL B-528 | WP_077843817.1 |
| CLBEIC contig000055, whole genome shotgun sequence | |
| Clostridium beijerinckii isolate C. beijerinckii DSM 6423 genome | WP_077843817.1 |
| assembly, chromosome: I | |
| Clostridium beijerinckii strain NRRL B-596 CLOBE_contig000006, | WP_077854102.1 |
| whole genome shotgun sequence | |
| Clostridium sp. BL-8 CLOBL_contig000019, whole genome | WP_077858634.1 |
| shotgun sequence | |
| Ruminococcus sp. HUN007 | WP_044974746.1 |
| CC97DRAFT_scf7180000000020_quiver.2_C, whole genome | |
| shotgun sequence | |
| Siculibacillus lacustris strain SA-279 scaffold_6, whole genome | WP_131307354.1 |
| shotgun sequence | |
| Pelosinus sp. UFO1, complete genome | WP_038671833.1 |
| Pectinatus cerevisiiphilus strain DSM 20467 Ga0244680_115, | WP_132550764.1 |
| whole genome shotgun sequence | |
| Clostridium tyrobutyricum isolate MGYG-HGUT-00125, whole | WP_017894495.1 |
| genome shotgun sequence | |
| Dendrosporobacter quercicolus strain DSM 1736, whole genome | WP_092071673.1 |
| shotgun sequence | |
| Rhodopseudomonas palustris strain YSC3 chromosome, complete | WP_107355474.1 |
| genome | |
| Sporomusaceae bacterium strain FL31 scf_SPFL3102_001, whole | WP_127032901.1 |
| genome shotgun sequence | |
| Sporomusaceae bacterium FL31 scf_SPFL3101_011, whole genome | WP_127032901.1 |
| shotgun sequence | |
| Ruminiclostridium hungatei strain DSM 14427 | WP_080066006.1 |
| CLHUN contig000028, whole genome shotgun sequence | |
| Propionispora vibrioides strain DSM 13305, whole genome shotgun | WP_091748359.1 |
| sequence | |
| Paenibacillus durus ATCC 35681, complete genome | WP_025700548.1 |
| Rhodopseudomonas palustris strain PS3 chromosome, complete | WP_107344318.1 |
| genome | |
| Sporomusa sp. KB1 SalpaDRAFT_Scaffold1.2, whole genome | WP_145096803.1 |
| shotgun sequence | |
| Propionispora sp. 2/2-37, whole genome shotgun sequence | WP_054261136.1 |
| Clostridium pasteurianum strain W5 contig00122, whole genome | WP_003444628.1 |
| shotgun sequence | |
| Clostridium sp. BNL 1100, complete genome | WP_014313541.1 |
| Paenibacillus stellifer strain DSM 14472, complete genome | WP_038694491.1 |
| Ruminiclostridium josui JCM 17888 | WP_024834404.1 |
| K412DRAFT_scf7180000000007_quiver.2_C, whole genome | |
| shotgun sequence | |
| Rhodopseudomonas palustris strain ELI 1980 Contig20, whole | WP_011158187.1 |
| genome shotgun sequence | |
| Rhodopseudomonas palustris CGA009 complete genome | WP_011158187.1 |
| Rhodopseudomonas palustris TIE-1, complete genome | WP_012496076.1 |
| Clostridium chromiireducens strain C1 Scaffold1, whole genome | WP_119365463.1 |
| shotgun sequence | |
| Rhodomicrobium sp. JA980 | WP_127077349.1 |
| NODE_13_length_1721687_cov_26.857853, whole genome | |
| shotgun sequence | |
| Clostridium tyrobutyricum strain Cirm BIA 2237 chromosome | WP_017894495.1 |
| Paenibacillus sabinae T27, complete genome | WP_025336406.1 |
| Clostridium pasteurianum DSM 525 = ATCC 6013 ctg1, whole | WP_003444628.1 |
| genome shotgun sequence | |
| Clostridium ljungdahlii DSM 13528, complete genome | WP_081442103.1 |
| Clostridium autoethanogenum DSM 10061, complete genome | WP_023161824.1 |
| Clostridium autoethanogenum DSM 10061, complete genome | WP_023161824.1 |
| Clostridium pasteurianum strain M150B, complete genome | WP_003444628.1 |
| Clostridium pasteurianum DSM 525 = ATCC 6013, complete | WP_003444628.1 |
| genome | |
| Clostridium pasteurianum DSM 525 = ATCC 6013, complete | WP_003444628.1 |
| genome | |
| Clostridium pasteurianum DSM 525 = ATCC 6013, complete | WP_003444628.1 |
| genome | |
| Clostridium pasteurianum BCI, complete genome | WP_015614356.1 |
| Clostridium sp. DL-VIII chromosome, whole genome shotgun | WP_009172466.1 |
| sequence | |

In some embodiments, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 8 (MarK).

SEQ ID NO: 8
PDAESRSQVTAKAAPPPAPKTNSIEQVRYICSIGAMHSAS
AIPRVIPITHCGPGCADKQFMNVAFYNGFQGGGYGGGAVV
PSTNATEREVVFGGAERLDELIGASLQVLDADLFVVLTGC
IPDLVGDDIGSVVGPYQKRGVPIVYAETGGFRGNNFTGHE
LVTKAIIDQFVGDYDAERDGAREPHTVNVWSLLPYHNTFW
RGDLTEIKRLLEGIGLKVNILFGPQSAGVAEWKAIPRAGF
NLVLSPWLGLDTARHLDRKYGQPTLHRPIIPIGAKETGAF
LREVAAFAGLDSAVVEAFITAEEAVYYRYLEDFTDFYAEY
WWGLPAKFAVIGDSAYNLALTKFLVNQLGLIPGLQIITDN
PPEEVREDIRAHYHAIADDVATDVSFEEDSYTIHQKIRAT
DFGHKAPILFGTTWERDLAKELKGAIVEVGFPASYEVVLS
RSYLGYRGALTLLEKIYTTTVSASA

In some embodiments, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 8. In some embodiments, the one or more genes of a methylthio-alkane reductase complex comprise a gene encoding a protein of SEQ ID NO: 8. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

The art is familiar with the methods and techniques used to identify other methylthio-alkane reductase genes and nucleotide sequences.

### Methionine Salvage Pathways

In some embodiments, the one or more genes of a methionine salvage pathway comprise one or more genes of a dihydroxyacetone phosphate (DHAP) shunt pathway. In some embodiments, the one or more genes of a DHAP shunt pathway comprise 5′-methylthioadenosine phosphorylase (mtnP), methylthioadenosine nucleosidase (mtn1), 5-methylthioribose kinase (mtnK), 5-methylthioribose-1-phosphate isomerase (mtnA), 5-methylthioribulose-1-phosphate aldolase (ald2), or combinations thereof.

In some embodiments, the one or more genes of a methionine salvage pathway comprises mtnP. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an mtnP gene associated with an accession number found in Table 4 below:

TABLE 4
Representative MtnP Genes

| Whole Genome Sequence of Origin | Accession Number |
|---|---|
| Pararhodospirillum oryzae strain NBRC 107573 sequence093, | WP_147164570.1 |
| whole genome shotgun sequence | |
| Rhodospirillum photometricum DSM 122 draft genome sequence | WP_041796869.1 |
| Rhodospirillum rubrum ATCC 11170 chromosome, complete | YP 425453.1 |
| genome | |
| Rhodospirillum rubrum F11, complete genome | YP 425453.1 |
| Rhodomicrobium udaipurense JA643 contig00206, whole genome | WP_037236245.1 |
| shotgun sequence | |
| Phaeospirillum fulvum MGU-K5 contig00054, whole genome | WP_039852757.1 |
| shotgun sequence | |
| Rhodoblastus sphagnicola strain DSM 16996 scaffold0018, whole | WP_104509034.1 |
| genome shotgun sequence | |
| Rhodoblastus acidophilus strain DSM 137 | WP_088521605.1 |
| NODE_116_length_9951_cov_47.3758, whole genome shotgun | |
| sequence | |
| Rhodoblastus acidophilus strain DSM 137, whole genome shotgun | WP_088521605.1 |
| sequence | |
| Rhodoblastus acidophilus strain DSM 137 scaffold0022, whole | WP_088521605.1 |
| genome shotgun sequence | |
| Rhodomicrobium sp. JA980 | WP_127078434.1 |
| NODE_3_length_364448_cov_26.852217, whole genome shotgun | |
| sequence | |
| Rhodomicrobium vannielii ATCC 17100, complete genome | WP_013421027.1 |
| Phaeospirillum fulvum strain DSM 13234, whole genome shotgun | WP_074767101.1 |
| sequence | |
| Rhodopseudomonas palustris strain 2.1.37 scaffold 36, whole | WP_011160353.1 |
| genome shotgun sequence | |
| Rhodopseudomonas palustris strain DSM 126 scaffold0001, whole | WP_011160353.1 |
| genome shotgun sequence | |
| Rhodopseudomonas palustris strain RI | WP_012497916.1 |
| NODE_28_length_158663_cov_40.885563, whole genome shotgun | |
| sequence | |
| Rhodopseudomonas sp. AAP120 AAP120_Contigs_11, whole | WP_054163535.1 |
| genome shotgun sequence | |
| Blastochloris viridis genome assembly Blastochloris viridis genome, | WP_055037880.1 |
| chromosome : I | |
| Blastochloris viridis strain ATCC 19567, complete genome | WP_055037880.1 |
| Blastochloris viridis DNA, complete genome, strain: DSM 133 | WP_055037880.1 |
| Rhodopseudomonas palustris strain RI | WP_012497916.1 |
| NODE_7_length_89266_cov_41.693230, whole genome shotgun | |
| sequence | |
| Rhodopseudomonas palustris strain DSM 126 scaffold0020, whole | WP_011160353.1 |
| genome shotgun sequence | |
| Rhodopseudomonas palustris strain 2.1.37 scaffold_3, whole | WP_011160353.1 |
| genome shotgun sequence | |
| Pelosinus sp. UFO1, complete genome | WP_038670973.1 |
| Pectinatus cerevisiiphilus strain DSM 20467 Ga0244680_115, | WP_132547855.1 |
| whole genome shotgun sequence | |
| Dendrosporobacter quercicolus strain DSM 1736, whole genome | WP_092067978.1 |
| shotgun sequence | |
| Rhodopseudomonas palustris strain YSC3 chromosome, complete | WP_012497916.1 |
| genome | |
| Sporomusaceae bacterium strain FL31 scf_SPFL3102_001, whole | WP_127035521.1 |
| genome shotgun sequence | |
| Sporomusaceae bacterium FL31 scf_SPFL3101_011, whole genome | WP_127035521.1 |
| shotgun sequence | |
| Propionispora vibrioides strain DSM 13305, whole genome shotgun | WP_091743455.1 |
| sequence | |
| Rhodopseudomonas palustris strain PS3 chromosome, complete | WP_012497916.1 |
| genome | |
| Sporomusa sp. KB1 SalpaDRAFT_Scaffold1.2, whole genome | WP_145100679.1 |
| shotgun sequence | |
| Propionispora sp. 2/2-37, whole genome shotgun sequence | WP_054258442.1 |
| Rhodopseudomonas palustris strain ELI 1980 Contig20, whole | WP_012497916.1 |
| genome shotgun sequence | |
| Rhodopseudomonas palustris CGA009 complete genome | WP_011160353.1 |
| Rhodopseudomonas palustris TIE-1, complete genome | WP_012497916.1 |
| Rhodomicrobium sp. JA980 | WP_127078434.1 |
| NODE_13_length_1721687_cov_26.857853, whole genome | |
| shotgun sequence | |

The art, is familiar with the methods and techniques used to identify other 5′-methylthioadenosine phosphorylase genes and nucleotide sequences.

In some embodiments, the one or more genes of a methionine salvage pathway comprises mtnK. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an mtnK gene associated with an accession number found in Table 5 below:

TABLE S
Representative MtnK Genes

| Whole Genome Sequence of Origin | Accession Number |
|---|---|
| Plcomorphomonas carboxyditropha strain SVCO-16 | WP_100082576.1 |
| NODE_2_length_581917_cov_22.4871, whole genome shotgun | |
| sequence | |
| Clostridium coskatii strain PTA-10522 CLCOS_contig000056, | WP_063602508.1 |
| whole genome shotgun sequence | |
| Clostridium coskatii strain PS02 scaffold19_1_86601, whole | WP_063602508.1 |
| genome shotgun sequence | |
| Clostridium drakei strain SLI contig_79, whole genome shotgun | WP_032078141.1 |
| sequence | |
| Clostridium drakei strain SLI chromosome, complete genome | WP_032078141.1 |
| Clostridium scatologenes strain ATCC 25775, complete genome | WP_029160459.1 |
| Clostridium ljungdahlii strain ERI-2 scaffold7, whole genome | WP_063556411.1 |
| shotgun sequence | |
| Plcomorphomonas carboxyditropha strain SVCO-16 | WP_100082576.1 |
| NODE_13_length_137005_cov_21.4606, whole genome shotgun | |
| sequence | |
| Pleomorphomonas sp. CF100 Ga0189743_114, whole genome | WP_134185490.1 |
| shotgun sequence | |
| Pleomorphomonas koreensis DSM 23070 | WP_053239417.1 |
| H512DRAFT_scaffold00010.10_C, whole genome shotgun | |
| sequence | |
| Roseiarcus fermentans strain DSM 24875 Ga0244512_102, whole | WP_113887889.1 |
| genome shotgun sequence | |
| Siculibacillus lacustris strain SA-279 scaffold_6, whole genome | WP_131310263.1 |
| shotgun sequence | |
| Clostridium sp. BNL 1100, complete genome | WP_014312607.1 |
| Ruminiclostridium josui JCM 17888 | WP_024831705.1 |
| K412DRAFT_scf7180000000007_quiver.2_C, whole genome | |
| shotgun sequence | |

The art is familiar with the methods and techniques used to identify other 5-methylthioribose kinase genes and nucleotide sequences.

In some embodiments, the one or more genes of a methionine salvage pathway comprises mtnA. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an mtnA gene associated with an accession number found in Table 6 below:

TABLE 6
Representative MtnA Genes

| Whole Genome Sequence of Origin | Accession Number |
|---|---|
| Pararhodospirillum oryzae strain NBRC 107573 sequence093, | WP_147164571.1 |
| whole genome shotgun sequence | |
| Rhodospirillum photometricum DSM 122 draft genome sequence | WP_014414708.1 |
| Rhodospirillum rubrum ATCC 11170 chromosome, complete | YP 425452.1 |
| genome | |
| Rhodospirillum rubrum F11, complete genome | YP 425452.1 |
| Rhodomicrobium udaipurense JA643 contig00206, whole genome | WP_037235257.1 |
| shotgun sequence | |
| Phaeospirillum fulvum MGU-K5 contig00054, whole genome | WP_021132531.1 |
| shotgun sequence | |
| Rhodoblastus sphagnicola strain DSM 16996 scaffold0018, whole | WP_104510706.1 |
| genome shotgun sequence | |
| Rhodoblastus acidophilus strain DSM 137 | WP_088520013.1 |
| NODE_116_length_9951_cov_47.3758, whole genome shotgun | |
| sequence | |
| Rhodoblastus acidophilus strain DSM 137, whole genome shotgun | WP_088520013.1 |
| sequence | |
| Rhodoblastus acidophilus strain DSM 137 scaffold0022, whole | WP_088520013.1 |
| genome shotgun sequence | |
| Rhodomicrobium sp. JA980 | WP_127076269.1 |
| NODE_3_length_364448_cov_26.852217, whole genome shotgun | |
| sequence | |
| Pleomorphomonas carboxyditropha strain SVCO-16 | WP_100082575.1 |
| NODE_2_length_581917_cov_22.4871, whole genome shotgun | |
| sequence | |
| Rhodomicrobium vannielii ATCC 17100, complete genome | WP_013418665.1 |
| Phaeospirillum fulvum strain DSM 13234, whole genome shotgun | WP_074765673.1 |
| sequence | |
| Clostridium coskatii strain PTA-10522 CLCOS_contig000056, | WP_063602507.1 |
| whole genome shotgun sequence | |
| Clostridium coskatii strain PS02 scaffold19_1_86601, whole | WP_063602507.1 |
| genome shotgun sequence | |
| Clostridium drakei strain SLI contig_79, whole genome shotgun | WP_032078140.1 |
| sequence | |
| Clostridium drakei strain SLI chromosome, complete genome | WP_032078140.1 |
| Clostridium scatologenes strain ATCC 25775, complete genome | WP_029160460.1 |
| Rhodopseudomonas palustris strain 2.1.37 scaffold_36, whole | WP_011160352.1 |
| genome shotgun sequence | |
| Rhodopseudomonas palustris strain DSM 126 scaffold0001, whole | WP_011160352.1 |
| genome shotgun sequence | |
| Rhodopseudomonas palustris strain R1 | WP_119019938.1 |
| NODE_28_length_158663_cov_40.885563, whole genome shotgun | |
| sequence | |
| Rhodopseudomonas sp. AAP120 AAP120_Contigs_11, whole | WP_054163536.1 |
| genome shotgun sequence | |
| Blastochloris viridis genome assembly Blastochloris viridis genome, | WP_055038971.1 |
| chromosome : I | |
| Blastochloris viridis strain ATCC 19567, complete genome | WP_055038971.1 |
| Blastochloris viridis DNA, complete genome, strain: DSM 133 | WP_055038971.1 |
| Ruminococcaceae bacterium HV4-5-B5C, whole genome shotgun | WP_114172929.1 |
| sequence | |
| Clostridium ljungdahlii strain ERI-2 scaffold7, whole genome | WP_063556410.1 |
| shotgun sequence | |
| Rhodopseudomonas palustris strain R1 | WP_119019938.1 |
| NODE_7_length_89266_cov_41.693230, whole genome shotgun | |
| sequence | |
| Rhodopseudomonas palustris strain DSM 126 scaffold0020, whole | WP_011160352.1 |
| genome shotgun sequence | |
| Rhodopseudomonas palustris strain 2.1.37 scaffold_3, whole | WP_011160352.1 |
| genome shotgun sequence | |
| Plcomorphomonas carboxyditropha strain SVCO-16 | WP_100082575.1 |
| NODE_13_length_137005_cov_21.4606, whole genome shotgun | |
| sequence | |
| Pleomorphomonas koreensis DSM 23070 | WP_026781788.1 |
| H512DRAFT_scaffold00010.10_C, whole genome shotgun | |
| sequence | |
| Roseiarcus fermentans strain DSM 24875 Ga0244512_102, whole | WP_113887888.1 |
| genome shotgun sequence | |
| Siculibacillus lacustris strain SA-279 scaffold_6, whole genome | WP_131310262.1 |
| shotgun sequence | |
| Pelosinus sp. UFO1, complete genome | WP_038670971.1 |
| Dendrosporobacter quercicolus strain DSM 1736, whole genome | WP_092067976.1 |
| shotgun sequence | |
| Rhodopseudomonas palustris strain YSC3 chromosome, complete | WP_107357324.1 |
| genome | |
| Sporomusaceae bacterium strain FL31 scf_SPFL3102_001, whole | WP_127035519.1 |
| genome shotgun sequence | |
| Sporomusaceae bacterium FL31 scf_SPFL3101_011, whole genome | WP_127035519.1 |
| shotgun sequence | |
| Propionispora vibrioides strain DSM 13305, whole genome shotgun | WP_091743454.1 |
| sequence | |
| Rhodopseudomonas palustris strain PS3 chromosome, complete | WP_107346345.1 |
| genome | |
| Sporomusa sp. KB1 SalpaDRAFT_Scaffold1.2, whole genome | WP_145100683.1 |
| shotgun sequence | |
| Propionispora sp. 2/2-37, whole genome shotgun sequence | WP_054258443.1 |
| Clostridium sp. BNL 1100, complete genome | WP_014312608.1 |
| Ruminiclostridium josui JCM 17888 | WP_024831704.1 |
| K412DRAFT_scf7180000000007_quiver.2_C, whole genome | |
| shotgun sequence | |
| Rhodopseudomonas palustris strain ELI 1980 Contig20, whole | WP_119019938.1 |
| genome shotgun sequence | |
| Rhodopseudomonas palustris CGA009 complete genome | WP_011160352.1 |
| Rhodopseudomonas palustris TIE-1, complete genome | WP_012497915.1 |
| Rhodomicrobium sp. JA980 | WP_127076269.1 |
| NODE_13_length_1721687_cov_26.857853, whole genome | |
| shotgun sequence | |

The art is familiar with the methods and techniques used to identify other 5-methylthioribose-1-P isomerase genes and nucleotide sequences.

In some embodiments, the one or more genes of a methionine salvage pathway comprises ald2. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an ald2 gene associated with an accession number found in Table 7 below:

TABLE 7
Representative Ald2 Genes

| Whole Genome Sequence of Origin | Accession Number |
|---|---|
| Rhodospirillum rubrum ATCC 11170 chromosome, complete | YP 425451.1 |
| genome | |
| Rhodospirillum rubrum F11, complete genome | YP 425451.1 |
| Rhodoblastus acidophilus strain DSM 137 | WP_088519984.1 |
| NODE_116_length_9951_cov_47.3758, whole genome shotgun | |
| sequence | |
| Rhodoblastus acidophilus strain DSM 137, whole genome shotgun | WP_088519984.1 |
| sequence | |
| Rhodoblastus acidophilus strain DSM 137 scaffold0022, whole | WP_088519984.1 |
| genome shotgun sequence | |
| Pleomorphomonas carboxyditropha strain SVCO-16 | WP_100082573.1 |
| NODE_2_length_581917_cov_22.4871, whole genome shotgun | |
| sequence | |
| Rhodopseudomonas palustris strain 2.1.37 scaffold_36, whole | WP_011160187.1 |
| genome shotgun sequence | |
| Rhodopseudomonas palustris strain DSM 126 scaffold0001, whole | WP_012497786.1 |
| genome shotgun sequence | |
| Rhodopseudomonas palustris strain R1 | WP_119019680.1 |
| NODE_28_length_158663_cov_40.885563, whole genome shotgun | |
| sequence | |
| Blastochloris viridis genome assembly Blastochloris viridis genome, | WP_055038972.1 |
| chromosome : I | |
| Blastochloris viridis strain ATCC 19567, complete genome | WP_055038972.1 |
| Blastochloris viridis DNA, complete genome, strain: DSM 133 | WP_055038972.1 |
| Rhodopseudomonas palustris strain R1 | WP_119019680.1 |
| NODE_7_length_89266_cov_41.693230, whole genome shotgun | |
| sequence | |
| Rhodopseudomonas palustris strain DSM 126 scaffold0020, whole | WP_012497786.1 |
| genome shotgun sequence | |
| Rhodopseudomonas palustris strain 2.1.37 scaffold_3, whole | WP_011160187.1 |
| genome shotgun sequence | |
| Pleomorphomonas carboxyditropha strain SVCO-16 | WP_100082573.1 |
| NODE_13_length_137005_cov_21.4606, whole genome shotgun | |
| sequence | |
| Pleomorphomonas sp. CF100 Ga0189743_114, whole genome | WP_134187437.1 |
| shotgun sequence | |
| Pleomorphomonas koreensis DSM 23070 | WP_053239475.1 |
| H512DRAFT_scaffold00010.10_C, whole genome shotgun | |
| sequence | |
| Roseiarcus fermentans strain DSM 24875 Ga0244512_102, whole | WP_113887630.1 |
| genome shotgun sequence | |
| Siculibacillus lacustris strain SA-279 scaffold_6, whole genome | WP_131310260.1 |
| shotgun sequence | |
| Pelosinus sp. UFO1, complete genome | WP_038670968.1 |
| Dendrosporobacter quercicolus strain DSM 1736, whole genome | WP_092067972.1 |
| shotgun sequence | |
| Rhodopseudomonas palustris strain YSC3 chromosome, complete | WP_107357124.1 |
| genome | |
| Sporomusaceae bacterium strain FL31 scf_SPFL3102_001, whole | WP_127035514.1 |
| genome shotgun sequence | |
| Sporomusaceae bacterium FL31 scf_SPFL3101_011, whole genome | WP_127035514.1 |
| shotgun sequence | |
| Propionispora vibrioides strain DSM 13305, whole genome shotgun | WP_091746076.1 |
| sequence | |
| Rhodopseudomonas palustris strain PS3 chromosome, complete | WP_107346191.1 |
| genome | |
| Propionispora sp. 2/2-37, whole genome shotgun sequence | WP_054261599.1 |
| Clostridium sp. BNL 1100, complete genome | WP_014312609.1 |
| Ruminiclostridium josui JCM 17888 | WP_024831703.1 |
| K412DRAFT_scf7180000000007_quiver.2_C, whole genome | |
| shotgun sequence | |
| Rhodopseudomonas palustris strain ELI 1980 Contig20, whole | WP_119019680.1 |
| genome shotgun sequence | |
| Rhodopseudomonas palustris CGA009 complete genome | WP_011160187.1 |
| Rhodopseudomonas palustris TIE-1, complete genome | WP_012497786.1 |
| Clostridium pasteurianum BCI, complete genome | WP_015616819.1 |

The art is familiar with the methods and techniques used to identify other 5-methylthioribulose-1-P aldolase genes and nucleotide sequences.

### Additional Genes

In some embodiments, the nucleic acid may encode one or more genes of a SAM hydrolase. In some embodiments, the one or more genes of a SAM hydrolase may be a non-naturally occurring, or exogenous, gene. In some embodiments, the SAM hydrolase may be derived from a coliphage virus. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

The art is familiar with the methods and techniques used to identify other SAM hydrolase genes and nucleotide sequences.

In some embodiments, the nucleic acid may encode one or more genes of a methanethiol methylase (mddA), a methionine gamma lyase (mgl), or combinations thereof. In some embodiments, the one or more genes of mddA, mgi, or combinations thereof, may be a non-naturally occurring, or exogenous, gene. In some embodiments, the one or more genes of mddA and/or mgl are derived from Rhodopseudomonal palsutris. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

The art is familiar with the methods and techniques used to identify other methanethiol methylase and/or methionine gamma lyase genes and nucleotide sequences.

In some embodiments, the nucleic acid may be codon optimized. In some embodiments, the one or more may be optionally and independently linked to a control element. In some embodiments, the control element comprises a promoter.

### Vectors

In another aspect, vectors are provided comprising one or more exogenous nucleic acid molecules encoding one or more genes of a methylthio-alkane reductase complex and one or more genes of a methionine salvage pathway. Vectors are also provided for use in the methods disclosed herein. For example, one or more of the vectors disclosed herein can be used to transform a microbial organism. Microbial organisms are also described transformed with or comprising one or more of the vectors described herein.

In some embodiments of the vectors described herein, the one or more genes of a methylthio-alkane reductase complex may comprise marB, marH, marD, marK, or combinations thereof.

In some embodiments of the vectors described herein, the one or more genes of a methylthio-alkane reductase complex comprise marB. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 1 (marB).

In some embodiments of the vectors described herein, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 1 (marB). In some embodiments, the one or more genese of a methylthio-alkane reductase complex comprise a nucleic acid sequence of SEQ ID NO: 1.

In some embodiments of the vectors described herein, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 2 (MarB).

In some embodiments of the vectors described herein, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 2. In some embodiments, the one or more genes of a methylthio-alkane reductase complex comprise a gene encoding a protein of SEQ ID NO: 2. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments of the vectors described herein, the one or more genes of a methylthio-alkane reductase complex comprise marH. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 3 (marH).

In some embodiments of the vectors described herein, the one or more genes of a methylthio-alkane reductase complex comprise a nucleic acid sequence having 90%, 91%, 92%, 93% 94%, 95%, 96%, 97%, 98%, or 99% identity to the nucleic acid sequence of SEQ ID NO: 3. In some embodiments, the one or more genese of a methylthio-alkane reductase complex comprise a nucleic acid sequence of SEQ ID NO: 3.

In some embodiments of the vectors described herein, the one or more genes of a methylthio-alkane reductase complex comprise one or more marH genes associated with an accession number found in Table 1.

In some embodiments of the vectors described herein, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 4 (MarH).

In some embodiments, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 4. In some embodiments, the one or more genes of a methylthio-alkane reductase complex comprise a gene encoding a protein of SEQ ID NO: 4. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments of the vectors described herein, the one or more genes of a methylthio-alkane reductase complex comprise marD. In some embodiments, the one or more genes of a methylthio-alkane reductase complex comprise a nucleic acid sequence having at least 60%, at least 65%, at least 70%, at least 75%, at least 80%, at least 85%, at least 90%, or more identity to the nucleic acid sequence of SEQ ID NO: 5 (marD).

In some embodiments of the vectors described herein, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 5. In some embodiments, the one or more genes of a methylthio-alkane reductase complex comprise a nucleic acid sequence of SEQ ID No: 5.

In some embodiments of the vectors described herein, the one or more genes of a methylthio-alkane reductase complex comprise one or more marD genes associated with an accession number found in Table 2.

In some embodiments of the vectors described herein, the one or more genes of a methylthio-alkane reductase complex comprise a gene encoding a protein having at least 60%, at least 65%, at least 70%, at least 75%, at least 80%, at least 85%, at least 90%, or more identity to the peptide sequence of SEQ ID NO: 6 (MarD).

In some embodiments of the vectors described herein, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 6. In some embodiments, the one or more genes of a inethylthio-alkane reductase complex comprise a gene encoding a protein of SEQ ID NO: 6. In some embodiments, the gene is a wildtype version of the gene or encodes a wildtype form of the associated protein. In some embodiments, the gene is a mutant form of the gene or may encode a mutant form of the associated protein point mutant, loss of function mutation, missense mutation, deletion, or insertion of heterologous nucleic acid).

In some embodiments of the vectors described herein, the one or more genes of a methylthio-alkane reductase complex comprise marK. In some embodiments, the one or more genes of a methylthio-alkane reductase complex comprise a nucleic acid sequence haying at least 60%, at least 65%, at least 70%, at least 75%, at least 80%, at least 85%, at least 90%, or more identity to the nucleic acid sequence of SEQ ID NO: 7 (marK).

In some embodiments of the vectors described herein, the one or more genes of a methylthio-alkane reductase complex comprise a nucleic acid sequence having 90%, 91%, 92%, 93%, 94%, 95%, 96%, 97%, 98%, or 99% identity to the nucleic acid sequence of SEQ ID NO: 7. In some embodiments, the one or more genes of a methylthio-alkane reductase complex comprise a nucleic acid sequence of SEQ ID NO: 7.

In some embodiments of the vectors described herein, the one or more genes of a methylthio-alkane reductase complex comprise one or more marK genes associated with an accession number found in Table 3.

In some embodiments of the vectors described herein, the one or more genes of a methylthio-alkane reductase complex comprise a gene encoding a protein haying at least 60%, at least 65%, at least 70%, at least 75%, at least 80%, at least 85%, at least 90%, or more identity to the peptide sequence of SEQ ID NO: 8 (MarK).

In some embodiments of the vectors described herein, the one or more genes of a methylthio-alkane reductase complex comprise a gene encoding a protein having 90%, 91%, 92%, 93%, 94%, 95%, 96%, 97%, 98%, or 99% identity to the peptide sequence of SEQ ID NO: 8. In some embodiments, the one or more genes of a methylthio-alkane reductase complex comprise a gene encoding a protein of SEQ ID NO: 8. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments of the vectors described herein, the one or more genes of a methionine salvage pathway comprise one or more genes of a dihydroxyacetone phosphate (DHAP) shunt pathway. In some embodiments, the one or more genes of a DHAP shunt pathway comprise 5′-methylthioadenosine phosphorylase (mtnP), 5-methylthioribose kinase (mtnK), 5-methylthioribose-1-phosphate isomerase (mtnA), 5-methylthioribulose-1-phosphate aldolase (ald2), or combinations thereof.

In some embodiments of the vectors described herein, the one or more genes of a methionine salvage pathway comprises mtnP. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an mtnP gene associated with an accession number found in Table 4.

In some embodiments of the vectors described herein, the one or more genes of a methionine salvage pathway comprises mtnl. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments of the vectors described herein, the one or more genes of a methionine salvage pathway comprises mtnK. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an mtnK, gene associated with an accession number found in Table 5.

In some embodiments of the vectors described herein, the one or more genes of a methionine salvage pathway comprises mtnA. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an mtnA gene associated with an accession number found in Table 6.

In some embodiments of the vectors described herein, the one or more genes of a methionine salvage pathway comprises ald2. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid). In some embodiments, the one or more genes of a methionine salvage pathway comprises an ald2 gene associated with an accession number found in Table 7.

In some embodiments of the vectors described herein, the exogenous nucleic acid molecules may further encode one or more genes of a SAM hydrolase. In some embodiments, the one or more genes of a SAM hydrolase may be a non-naturally occurring, or exogenous, gene. In some embodiments, the SAM hydrolase may be derived from a coliphage virus. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid).

In some embodiments of the vectors described herein, the exogenous nucleic acid molecules may encode one or more genes of a methanethiol methylase (mddA), a methionine gamma lyase (mgl), or combinations thereof. In some embodiments, the one or more genes of mddA, mgl, or combinations thereof, may be a non-naturally occurring, or exogenous, gene. In some embodiments, the one or more genes of mddA and/or mgl are derived from Rhodopseudomonal palsutris. In some embodiments, the gene is a wildtype version of the gene or encodes a wildtype form of the associated protein. In some embodiments, the gene is a mutant form of the gene or may encode a mutant form of the associated protein (e.g. point mutant, loss of function mutation, missense mutation, deletion, or insertion of heterologous nucleic acid),

In some embodiments the one or more exogenous nucleic acid molecules are integrated into a gene expression cassette. In some embodiments, the gene expression cassette comprises one or more control elements. In some embodiments, the one or more exogenous nucleic acid molecules disclosed herein are operably linked to a control element. In some embodiments, the control element is a promoter. In some embodiments, the promoter may be constitutively active or inducibly active. In some embodiments, the promoter is constitutively active regardless of sulfate concentration, i.e., sulfate limitation is not required in order to induce expression of the gens found in the one or more exogenous nucleic acid molecules.

In some embodiments, the promoter comprises a nucleic acid sequence having at least 60%, at least 65%, at least 70%, at least 75%, at least 80%, at least 85%, at least 90%, or more identity to the sequence of SEQ ID NO: 9:

SEQ ID NO: 9
AAACCGCTTTAACCGCCATCCTGCGCTAAACGGCCGCCGG
CCCCCACCGGCGGCCGTTTTTTATTCGCCGCCCCTCCCCG
CGACGGGCTCCCTCGCCTTGGTGGCTTTTCATCCGGGGGG
GTGGCGCGCTAAGGTGCCCCACCCGCAAAAGGGTGAGCCA
GCCAGGAAGAGGGGAACAT

In some embodiments, the promoter comprises a nucleic acid sequence having 90%, 91%, 92%, 93%, 94%, 95%, 96%, 97%, 98%, or 99% identity to the nucleic acid sequence of SEQ ID NO: 9. In some embodiments, the promoter comprises a nucleic acid sequence of SEQ ID NO: 9.

In some embodiments, the promoter comprises a nucleic acid sequence having at least 60%, at least 65%, at least 70%, at least 75%, at least 80%, at least 85%, at least 90%, or more identity to the sequence of SEQ ID NO: 10:

SEQ ID NO: 10
GGGCATGGCGCGGATGATCCGCCCGCTCTCGGGCTCGCCA
CACGAGGTTTTCCGGGGTTTTCCGCTCCTTTCGGGGCAGA
ACACGCCGGATAACAAGGTCCGTCCCGACCTGGTCGGGTG
GACTTCTTACCGCGGTTCTTCACCGCGGTAGAGCAGCCGT
TCCCTGCGCGGATGCAGTGGAATGGTTTTCTGGGCAAGAA
TTAGGAGGTAGCACAT

In some embodiments, the promoter comprises a nucleic acid sequence having 90%, 91%, 92%, 93%, 94%, 95%, 96%, 97%, 98%, or 99% identity to the nucleic acid sequence of SEQ ID NO: 10. In some embodiments, the promoter comprises a nucleic acid sequence of SEQ ID NO: 10.

In another aspect, a non-naturally occurring organism is provided comprising a vector described herein.

### Methods of Use

In another aspect, methods of producing ethylene, ethane, and/or methane are provided comprising:

culturing a population of the non-naturally occurring microbial organism described herein in a culture medium comprising one or more carbon sources; and

recovering the ethylene, ethane, and/or methane.

In some embodiments, the methods described herein may be used in the production of ethylene. In some embodiments, the methods described herein may be used in the production of ethane. In some embodiments, the methods described herein may be used in the production of methane.

The term “carbon source” means a carbon source that a microbial organism described herein will metabolize to derive energy (e.g. monosaccharides, oligosaccharides, polysaccharides, alkanes, fatty acids, esters of fatty acids, monoglycerides, acetate, carbon dioxide, methanol, formaldehyde, formate or carbon-containing amines). The term “carbon source” refers to a carbon containing composition (e.g. compound, mixture of compounds) that an organism may metabolize for use by the organism or that may be used for organism viability. A “majority carbon source” refers to a carbon containing composition that accounts for greater than 50% of the available carbon sources for an organism (e.g. in a media, in a growth media, in a defined media for the organism, or in a defined media for producing ethylene, ethane, and/or methane by an organism) at a specified time (e.g. media when starting a culture, media in a bioreactor when growing the organism, or media when producing ethylene, ethane, and/or methane from the organism). In embodiments, an organism may be cultured using a medium comprising a majority carbon source selected from the group consisting of glucose, glycerol, xylose, fructose, mannose, ribose, sucrose, and lignocellusic biomass. In embodiments, an organism may be cultured using a medium comprising one or more carbon sources selected from the group consisting of glucose, fructose, sucrose, lactose, galactose, xylose, mannose, rhamnose, arabinose, glycerol, acetate, depolymerized sugar beet pulp, black liquor, corn starch, depolymerized cellulosic material, corn stover, sugar beet pulp, switchgrass, milk whey, molasses, potato, rice, sorghum, sugar cane, wheat, and mixtures thereof (e.g. mixtures of glycerol and glucose, mixtures of glucose and xylose, mixtures of fructose and glucose, mixtures of sucrose and depolymerized sugar beet pulp, black liquor, corn starch, depolymerized cellulosic material, corn stover, sugar beet pulp, switchgrass, milk whey, molasses, potato, rice, sorghum, sugar cane, and/or wheat). In some embodiments, an organism is cultured using a medium comprising one or more carbon sources selected from the group consisting of depolymerized sugar beet pulp, black liquor, corn starch, depolymerized cellulosic material, corn stover, sugar beet pulp, switchgrass, milk whey, molasses, potato, rice, sorghum, sugar cane, thick cane juice, sugar beet juice, and wheat. In some embodiments, an organism is cultured using a medium comprising lignocellulosic biomass. In some embodiments, carbon sources may be monosaccharides (e.g., glucose, fructose), disaccharides (e.g., lactose, sucrose), oligosaccharides, polysaccharides (e.g., starch, cellulose or mixtures thereof), sugar alcohols (e.g., glycerol) or mixtures from renewable feedstocks (e.g., cheese whey permeate, cornsteep liquor, sugar beet molasses, or barley malt). Additionally, carbon sources may include alkanes, thtty acids, esters of fatty acids, monoglycerides, diglycerides, triglycerides, phospholipids, various commercial sources of fatty acids including vegetable oils (e.g., soybean oil) or animal fats. In some embodiments, the culture medium may contain, in addition to the primary (or majority) carbon source, one or more secondary carbon sources. In some embodiments, the secondary carbon source comprises lignin or lignin derived aromatic compounds. In some embodiments, the secondary carbon source comprises lignin breakdown products.

In some embodiments, the one or more carbon sources may comprise biomass, for example lignocellulosic biomass. The term “biomass” refers to material produced by growth and/or propagation of cells. “Lignocellulosic biomass” is used according to it plain and ordinary meaning and refers to plant dry matter comprising carbohydrate (e.g. cellulose or hemicellulose) and polymer (e.g. lignin). Lignocellulosic biomass may include agricultural residues (e.g. corn stover or sugarcane bagasse), energy crops (e.g. poplar trees, willow, Miscanthus purpureum, Pennisetum purpureum, elephant grass, maize, Sudan grass, millet, white sweet clover, rapeseed, giant miscanthus, switchgrass, jatropha, Miscanthus giganteus, or sugarcane), wood residues (e.g. sawmill or papermill discard), or municipal paper waste.

In some embodiments, the one or more carbon sources may be selected from one or more in combination of: carbon dioxide and carbon monoxide, mono and disaccharide sugars, organic acids (for example, malate, succinate, pyruvate, and fumarate), volatile fatty acids (for example, formate, acetate, propionate, and butyrate), alcohols (for example, ethanol and glycerol), and cellulosic plant biomass including but not limited to corn stover, miscanthus, switchgrass.

A “growth media” or “growth medium” as used herein can be a solid, powder, or liquid mixture which comprises all or substantially all of the nutrients necessary to support the growth of an organism; various nutrient compositions are preferably prepared when particular species are being assayed. Amino acids, carbohydrates, minerals, vitamins and other elements known to those skilled in the art to be necessary for the growth of microbial organisms are provided in the medium. In one embodiment, the growth medium is liquid. In one embodiment, the growth medium is a production medium (for example, medium optionally containing higher concentrations of glucose and/or altered concentrations of nitrogen).

In some embodiments, the growth media is sufficiently deficient in or absent of sulfate.

In another aspect, a bioreactor is provided comprising a non-naturally occurring organism as described herein. Such bioreactors may be used in the methods described herein.

### EMBODIMENTS

Further embodiments of the present disclosure are provided as follows:
Embodiment 1: a non-naturally occurring microbial organism comprising a nucleic acid encoding one or more genes of a methylthio-alkane reductase complex and one or more genes of a methionine salvage pathway.
Embodiments 2: a non-naturally occurring microbial organism of embodiment 1, wherein the organism produces ethylene, ethane, methane, or combinations thereof.
Embodiment 3: the non-naturally occurring microbial organism of embodiment 2, wherein the organism produces ethylene.
Embodiment 4: the non-naturally occurring microbial organism of embodiment 2, wherein the organism produces ethane.
Embodiment 5: the non-naturally occurring microbial organism of embodiment 2, wherein the organism produces methane.
Embodiment 6: the non-naturally occurring microbial organism of any one of embodiments 1-5, wherein the one or more genes of a methylthio-alkane reductase complex comprise marB, marD, marK, or combinations thereof.
Embodiment 7: the non-naturally occurring microbial organism of any one of embodiments 1-6, wherein the one or more genes of a methylthio-alkane reductase complex comprise a nucleic acid sequence having at least 85% identity to the nucleic acid sequence of SEQ ID NO: 1.
Embodiment 8: the non-naturally occurring microbial organism of embodiment 7, wherein the one or more genes of a methylthio-alkane reductase complex comprise a nucleic acid sequence of SEQ ID NO: 1.
Embodiment 9: the non-naturally occurring microbial organism of any one of embodiments 1-8, wherein the one or more genes of a methylthio-alkane reductase complex comprise a nucleic acid sequence having at least 85% identity to the nucleic acid sequence of SEQ ID NO: 3.
Embodiment 10: the non-naturally occurring microbial organism of embodiment 9, wherein the one or more genes of a methylthio-alkane reductase complex comprise a nucleic acid sequence of SEQ ID NO: 3.
Embodiment 11: the non-naturally occurring microbial organism of any one of embodiments 1-10, wherein the one or more genes of a methylthio-alkane reductase complex comprise a nucleic acid sequence having at least 85% identity to the nucleic acid sequence of SEQ ID NO: 5.
Embodiment 12: the non-naturally occurring microbial organism of embodiment 11, wherein the one or more genes of a methylthio-alkane reductase complex comprise a nucleic acid sequence of SEQ ID NO: 5.
Embodiment 13: the non-naturally occurring microbial organism of any one of embodiments 1-12, wherein the one or more genes of a methylthio-alkane reductase comprise a nucleic acid sequence having at least 85% identity to the nucleic acid sequence of SEQ ID NO: 7.
Embodiment 14: the non-naturally occurring microbial organism of embodiment 13, wherein the one or more genes of a methylthio-alkane reductase complex comprise a nucleic acid sequence of SEQ ID NO: 7.
Embodiment 15: the non-naturally occurring organism of any one of embodiments 1-14, wherein the one or more genes of a methionine salvage pathway comprise one or more genes of a dihydroxyacetone phosphate (DHAP) shunt pathway.
Embodiment 16: the non-naturally occurring organism of embodiment 15, wherein the one or more genes of a DHAP shunt pathway comprise 5′-methylthioadenosine phosphorylase (mtnP), methylthioadenosine nucleosidase (mtn1), 5-methylthioribose kinase (mtnK), 5-methylthioribose-1-phosphate isomerase (mtnA), 5-methylthioribulose-1-phosphate aldolase (ald2), or combinations thereof.
Embodiment 17: the non-naturally occurring organism of embodiment 16, wherein the one or more genes of a DHAP shunt pathway comprise mtnP.
Embodiment 18: the non-naturally occurring organism of embodiment 16, wherein the one or more genes of a DHAP shunt pathway comprise intni and mtnK.
Embodiment 19: the non-naturally occurring organism of any one of embodiments 16-18, wherein the one or more genes of a DHAP shunt pathway comprise mtnA.
Embodiment 20: the non-naturally occurring organism of any one of embodiments 16-19, wherein the one or more genes of a DHAP shunt pathway comprise ald2.
Embodiment 21: the non-naturally occurring microbial organism of any one of embodiments 1-20, wherein the nucleic acid further encodes one or more genes of a SAM hydrolase.
Embodiment 22: the non-naturally occurring microbial organism of any one of embodiments 1-10, wherein the nucleic acid further encodes one or more genes of a methanethiol methylase (mddA), a methionine gamma lyase, or combinations thereof.
Embodiment 23: the non-naturally occurring microbial organism of any one of embodiments 1-22, wherein the nucleic acid is codon optimized.
Embodiment 24: the non-naturally occurring microbial organism of any one of embodiments 1-23, wherein the nucleic acid is integrated into the genome of the organism.
Embodiment 25: the non-naturally occurring microbial organism of any one of embodiments 1-23, wherein the nucleic acid is episomally integrated into a plasmid.
Embodiment 26: a non-naturally occurring microbial organism, wherein the organism is an anaerobic organism which produces ethylene, ethane, and/or methane using a methylthio-alkane reductase complex and a methionine salvage pathway, and wherein the organism has been optimized for producing ethylene, ethane, and/or methane with one or more non-naturally occurring genes.
Embodiment 27: the non-naturally occurring microbial organism of embodiment 26, wherein the one or more non-naturally occurring genes comprise one or more genes of a SAM hydrolase.
Embodiment 28: the non-naturally occurring microbial organism of embodiment 26, wherein the one or more non-naturally occurring genes comprise one or more genes of a methanethiol methylase (mddA), a methionine gamma lyase (mgl), or combinations thereof.
Embodiment 29: the non-naturally occurring microbial organism of any one of embodiments 26-28, wherein the one or more non-naturally occurring genes are integrated into the genome of the organism.
Embodiment 30: the non-naturally occurring microbial organism of any one of embodiments 26-28, wherein the one or more non-naturally occurring genes are episomally expressed from a plasmic.
Embodiment 31: the non-naturally occurring microbial organism of any one of embodiments 26-30, wherein the one or more non-naturally occurring genes are codon optimized.
Embodiment 32: a method of producing ethylene, ethane, and/or methane comprising:
   culturing a population of the non-naturally occurring microbial organism of any one of embodiments 1-31 in a culture medium comprising one or more carbon sources; and
   recovering the ethylene, ethane, and/or methane.
Embodiment 33: the method of embodiment 32, wherein the one or more carbon sources comprise carbon dioxide, carbon monoxide, an organic acid, a volatile fatty acid, an alcohol, cellulosic plant mass, or combinations thereof.
Embodiment 34: the method of embodiment 32 or 33, wherein the one or more carbon sources comprise carbon dioxide, carbon monoxide, malate, succinate, pyruvate, fumarate, formate, acetate, propionate, butyrate, ethanol, glycerol, corn stover, miscanthus, or switchgrass.
Embodiment 35: the method of any one of embodiments 32-34, wherein the one or more carbon sources comprise corn stover.
Embodiment 36: the method of embodiment 32, wherein the one or more carbon sources comprise lignoceliulosic biomass.
Embodiment 3: the method of any one of embodiments 32-36, wherein the population is cultured in the absence of sulfate.
Embodiment 38: a bioreactor comprising the non-naturally occurring microbial organism of any one of embodiments 1-31.

Embodiment 39: a vector comprising: one or more exogenous nucleic acid molecules encoding one or more genes of a methylthio-alkane reductase complex and one or more genes of a methionine salvage pathway.
Embodiment 40: the vector of embodiment 39, wherein the one or more genes of a methylthio-alkane reductase complex comprise marB, marH, marD, marK, or combinations thereof.
Embodiment 41: the vector of embodiment 39 or embodiment 40, wherein the one or more genes of a methylthio-alkane reductase complex comprise a nucleic acid sequence having at least 85% identity to the nucleic acid sequence of SEQ ID NO: 1.
Embodiment 42: the vector of embodiment 41, wherein the one or more genes of a methylthio-alkane reductase complex comprise a nucleic acid sequence of SEQ ID NO: 1.
Embodiment 43: the vector of any one of embodiments 39-42, wherein the one or more genes of a methylthio-alkane reductase complex comprise a nucleic acid sequence having at least 85% identity to the nucleic acid sequence of SEQ ID NO: 3.
Embodiment 44: the vector of embodiment 43, wherein the one or more genes of a methylthio-alkane reductase complex comprise a nucleic acid sequence of SEQ ID NO: 3.
Embodiment 45: the vector of any one of embodiments 39-44, wherein the one or more genes of a methylthio-alkane reductase complex comprise a nucleic acid sequence having at least 85% identity to the nucleic acid sequence of SEQ ID NO: 5.
Embodiment 46: the vector of embodiment 43, wherein the one or more genes of a methylthio-alkane reductase complex comprise a nucleic acid sequence of SEQ ID NO: 5.
Embodiment 47: the vector of any one of embodiments 39-46, wherein the one or more genes of a methylthio-alkane reductase comprise a nucleic acid sequence having at least 85% identity to the nucleic acid sequence of SEQ ID NO: 7.
Embodiment 48: the vector of embodiment 47, wherein the one or more genes of a methylthio-alkane reductase comprise a nucleic acid sequence of SEQ ID NO: 7.
Embodiment 49: the vector of any one of embodiments 39-48, wherein the one or more genes of a methionine salvage pathway comprise one or more genes of a dihydroxyacetone phosphate (DHAP) shunt pathway.
Embodiment 50: the vector of embodiment 49, wherein the one or more genes of a DHAP shunt pathway comprise 5′-methylthioadenosine phosphorylase (mtnP), 5-methylthioribose kinase (mtnK) 5-methylthioribose-1-phosphate isomerase (mtnA), 5-methylthioribulose-1-phosphate aldolase (ald2), alcohol dehydrogenase (adh), or combinations thereof.
Embodiment 51: the vector of embodiment 50, wherein the one or more genes of a DHAP shunt pathway comprise mtnP.
Embodiment 52: the vector of embodiment 50, wherein the one or more genes of a DHAP shunt pathway comprise mtn1 and mtnK.
Embodiment 53: the vector of any one of embodiments 50-52, wherein the one or more genes of a DHAP shunt pathway comprise mtnA.
Embodiment 54: the vector of any one of embodiments 50-53, wherein the one or more genes of a DHAP shunt pathway comprise ald2.
Embodiment 55: the vector of any one of embodiments 39-54, wherein the one or more exogenous nucleic acid molecules further encode one or more genes of a SAM hydrolase.
Embodiment 56: the vector of any one of embodiments 39-55, wherein the one or more exogenous nucleic acid molecules further encode one or more genes of a methanethiol methylase (mddA), a methionine gamma lyase (mgl), or combinations thereof.
Embodiment 57: the vector of any one of embodiments 39-56, wherein the one or more genes are integrated into a gene expression cassette.
Embodiment 58: the vector of embodiment 57, wherein the gene expression cassette comprises a promoter.
Embodiment 59: the vector of embodiment 58, wherein the promoter comprises a nucleic acid sequence having at least 85% identity to the nucleic acid sequence of SEQ ID NO: 9.
Embodiment 60: the vector of embodiment 59, wherein the promoter comprises a nucleic acid sequence of SEQ ID NO: 9.
Embodiment 61: the vector of embodiment 58, wherein the promoter comprises a nucleic acid sequence having at least 85% identity to the nucleic acid sequence of SEQ ID NO: 10.
Embodiment 62: the vector of embodiment 61, wherein the promoter comprises a nucleic acid sequence of SEQ ID NO: 10.
Embodiment 63: the vector of any one of embodiments 39-62, wherein the one or more genes have been codon optimized.
Embodiment 64: a non-naturally occurring organism comprising a vector of any one of embodiments 39-63.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### A Nitrogenase-Like Enzyme System Catalyzes Methionine, Ethylene, Ethane, and Methane Biogenesis

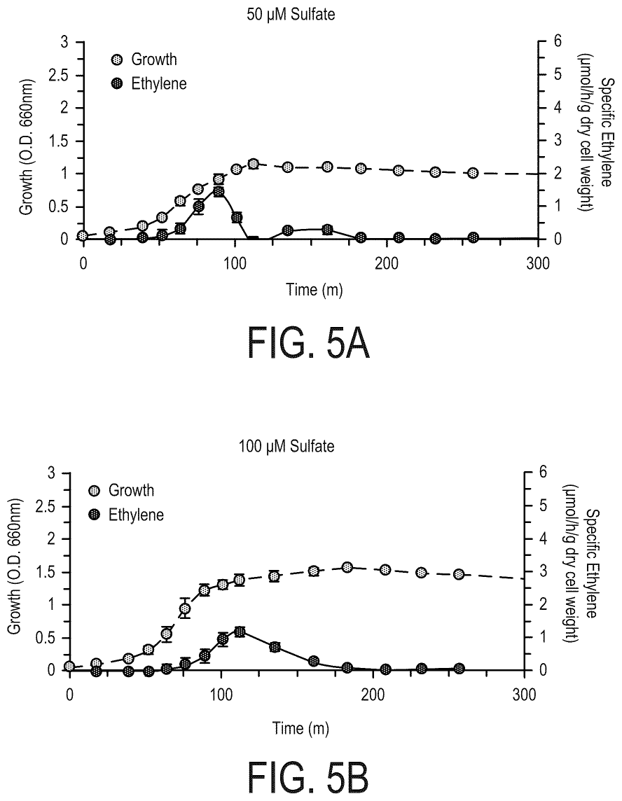

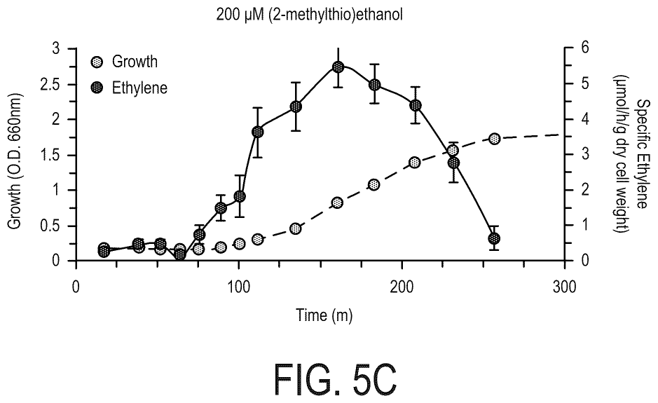

R. rubrum was grown under conditions for ethylene induction (50 μM limiting sulfate or 1 mM MT-EtOH as sole S-source) and ethylene repression (1 mM sulfate) (FIGS. 5A-5C) (10). Proteomics differential abundance analysis identified multiple proteins that increased over 20-fold in proteomes from induced versus repressed cells (FIG. 1B). Among these were enzymes involved in cysteine and methionine metabolism: homoserine/serine: O-acetyltransferase (CysE), O-acetyl-L-homoserine sulfhydrylase, cystathionine beta-synthase, and cystathionine gamma-lyase (FIGS. 1A-1C, reactions 2, 3, 6, 7, respectively).

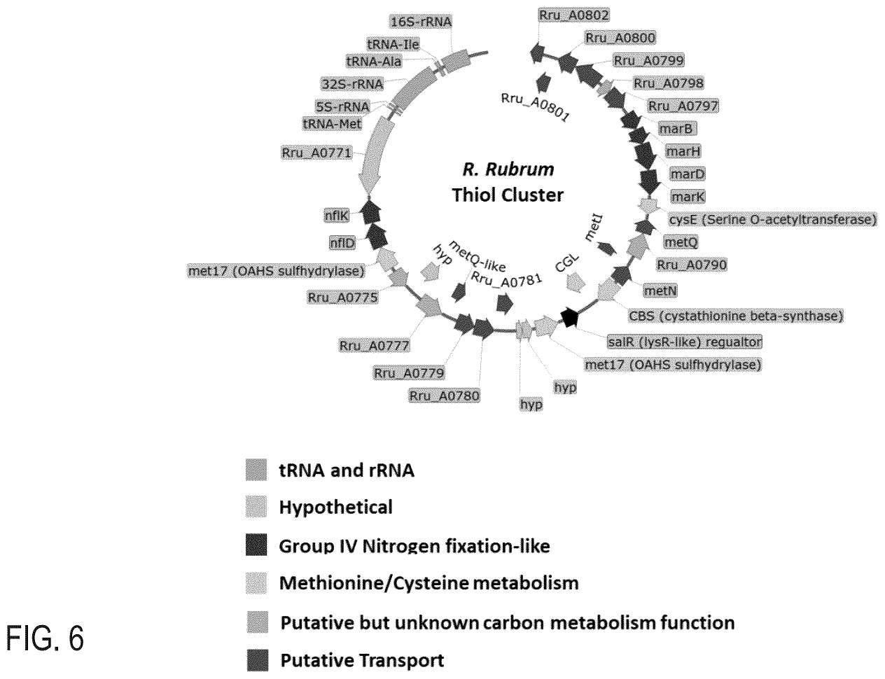

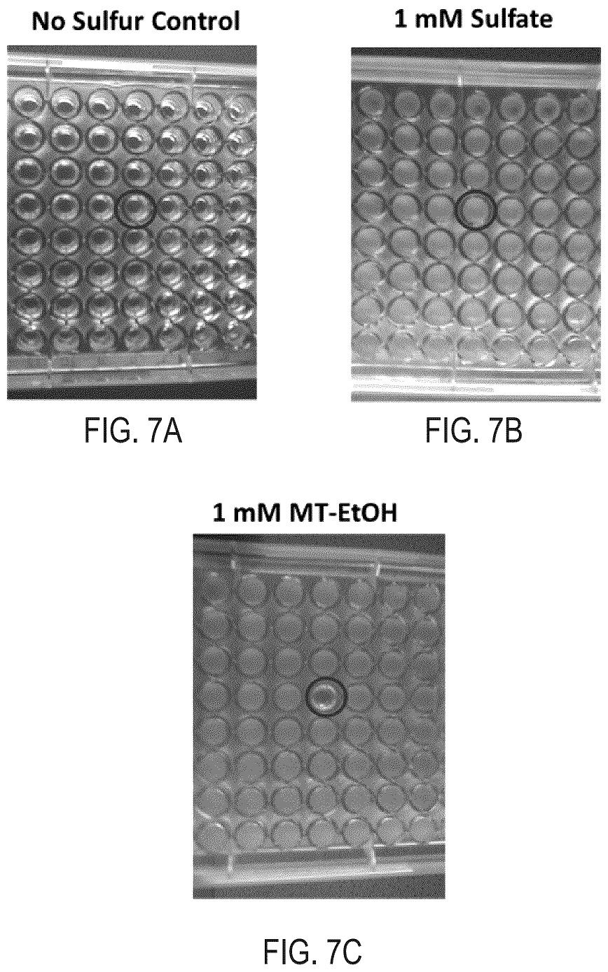

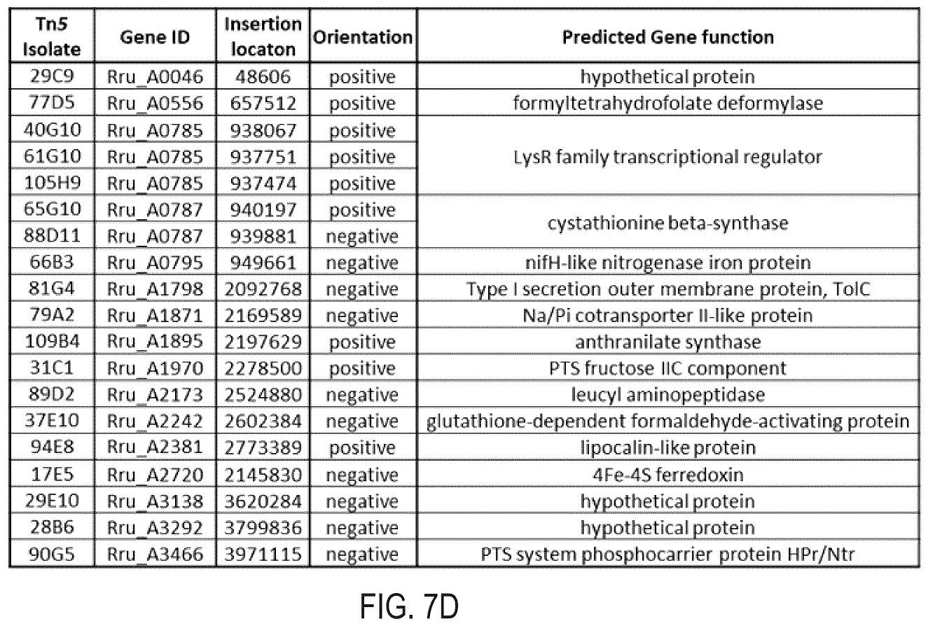

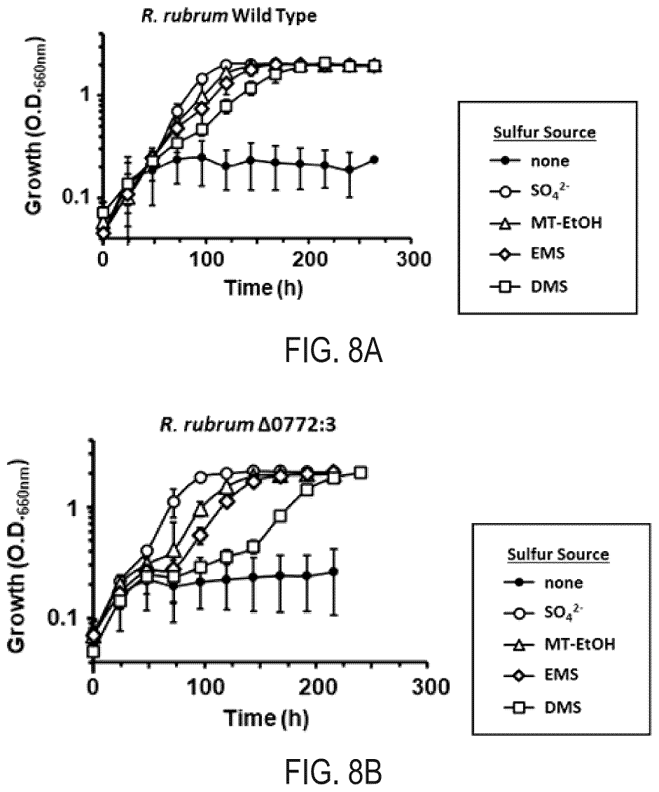

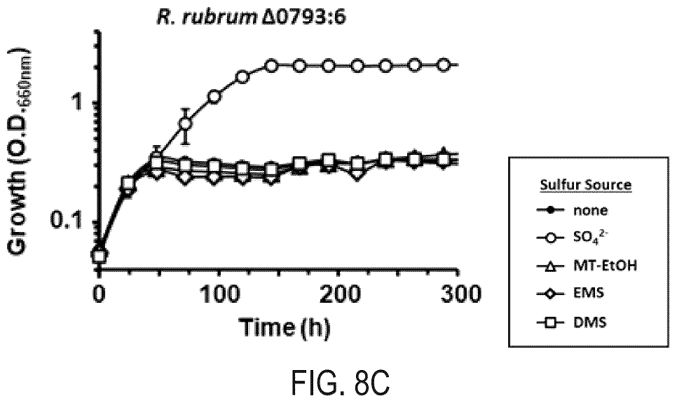

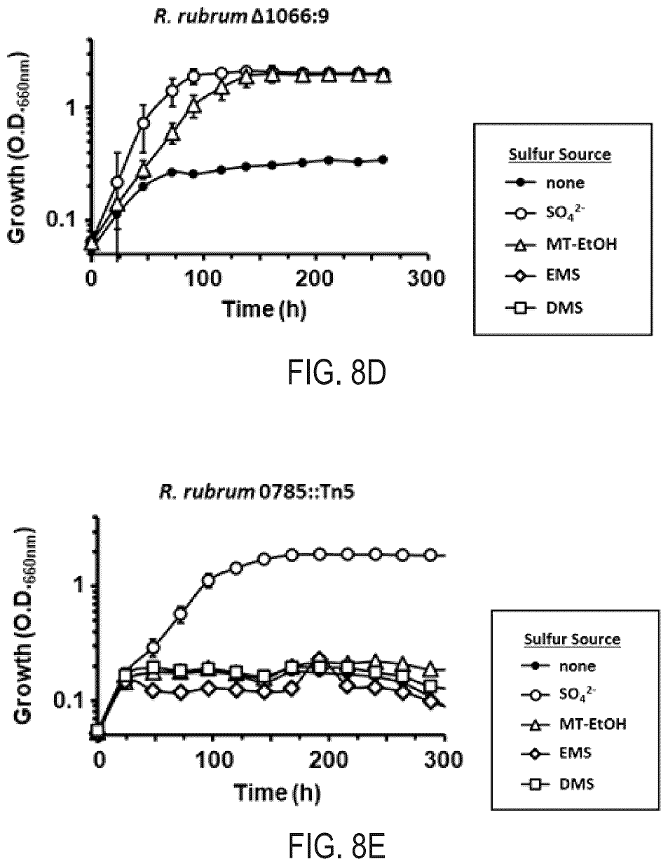

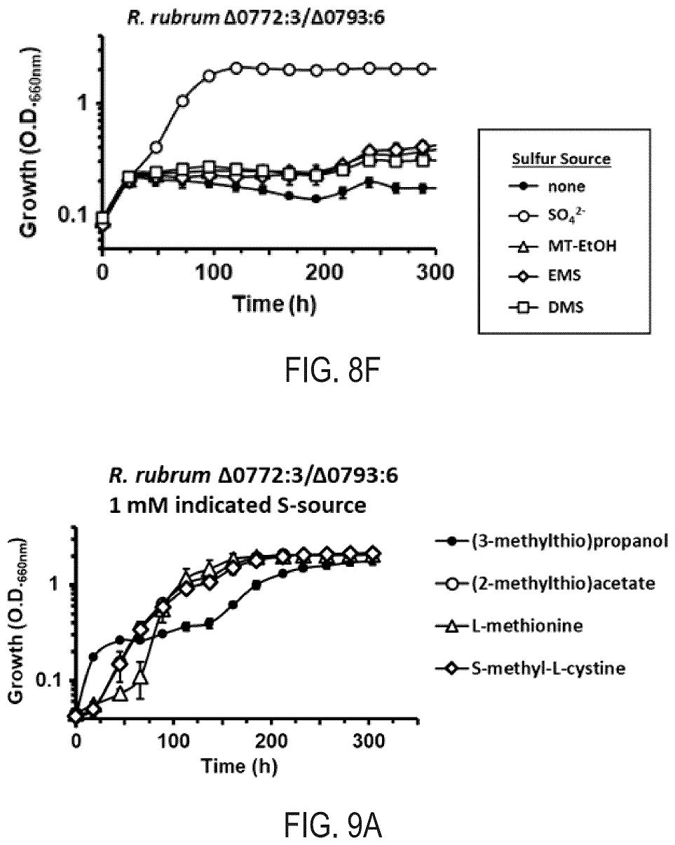

Several proteins previously identified as NFL sequences of unknown function (8,9) showed some of the highest increases in abundance under ethylene inducing conditions (FIG. 1B, Rru_A0772-Rru_A0773 and Rru_A0793-Rru_A0796, see FIG. 6 for gene organization). In addition, there was also a large increase in abundance of proteins likely involved in iron-sulfur cluster metabolism; NifS cysteine desulfurase and a putative Fe₄—S₄ scaffold protein (FIG. 1B, Rru_A1068-Rru_A1069). This appears analogous to the Azotobacter vinelandii NifUS system for synthesis of nitrogenase-destined iron-sulfur clusters from cysteine (12). However, the precise iron-sulfur cluster assembly pathway in R. rubrum is unknown. The involvement of the nitrogenase-like system in ethylene production was further bolstered by the R. rubrum transposon mutant strain WRdht-66B3, possessing an inactivated gene encoding a putative nitrogenase reductase-like iron protein (Rru_A0795; FIG. 1B). This and other mutants identified in a random mutagenesis screen were unable to grow anaerobically in the presence of MT-EtOH as sole S-source but could still grow utilizing sulfate, indicatirig a defect in the ethylene-producing pathway (FIGS. 7A-7D). Consistent with the Tn5 mutagenesis results, specific deletion of NFL gene cluster Rru_A0793-Rru_A0796 rendered R rubrum incapable of growth or production of ethylene above basal levels with MT-EtOH as sole S-source (FIGS. 2A-B and FIG. 8C). This result confirmed that the putative nitrogenase-like system encoded by NFL gene duster Rru_A0793-Rru_A0796 was essential for assimilating sulfur from MT-EtOH to produce ethylene and methionine.

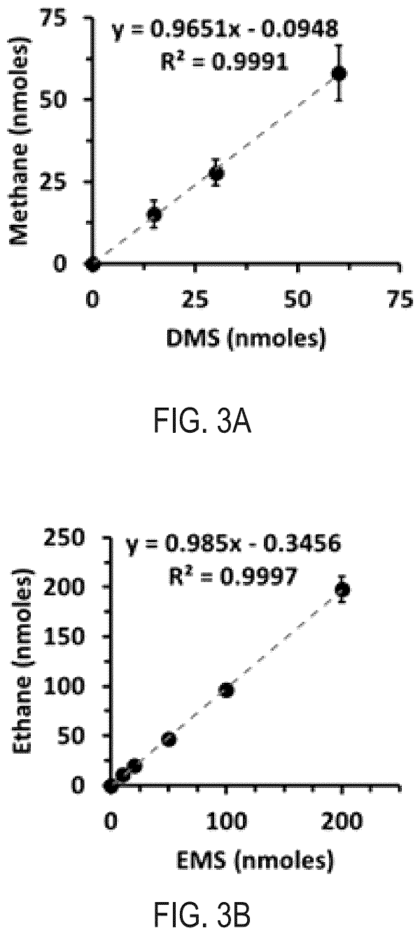

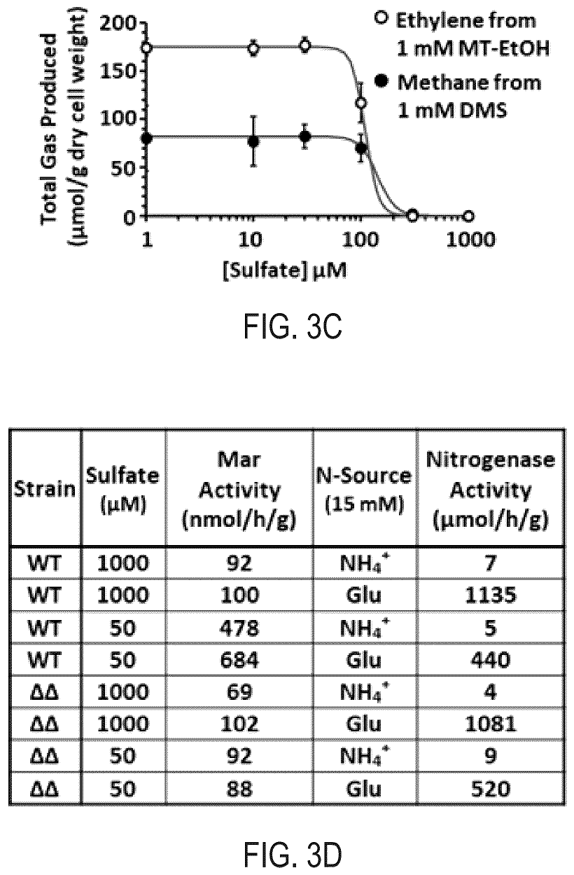

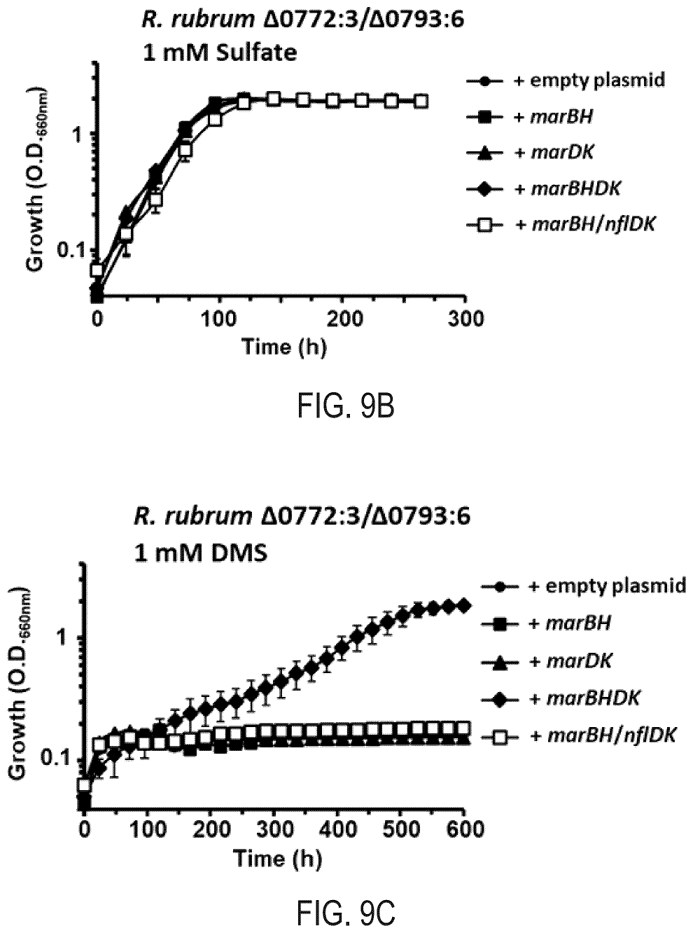

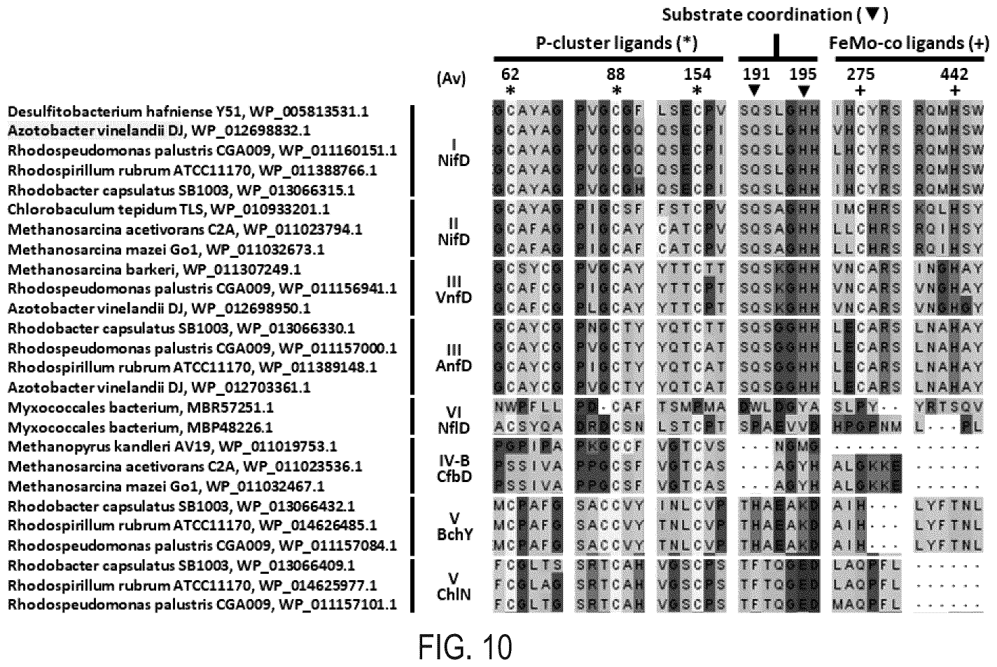

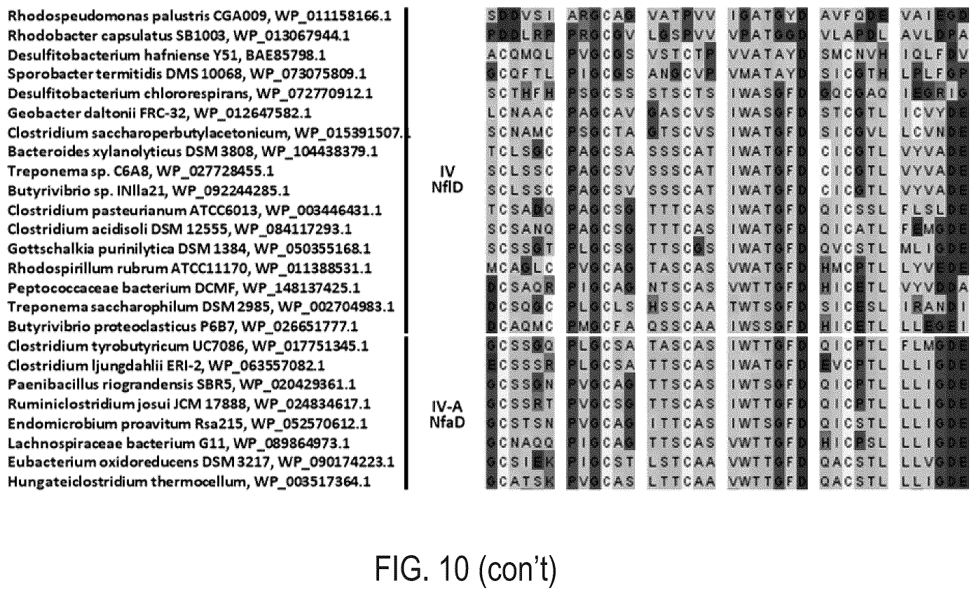

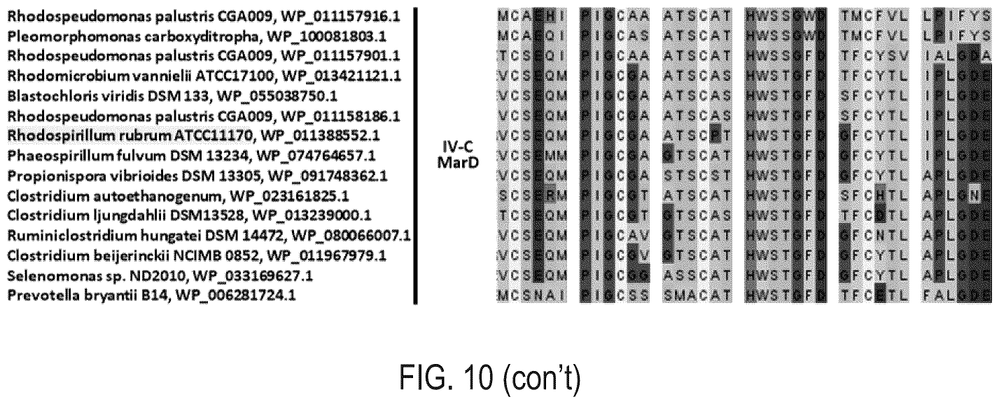

Other biologically relevant volatile organic sulfur compounds (VOSCs) were then tested for utilization by this putative nitrogenase-like enzyme system (FIG. 2A-B and FIG. 9A). In addition to MT-EtOH, VOSC utilization with concomitant hydrocarbon production was specific to dimethyl sulfide (DMS), the most abundant environmental VOSC, and ethyl methyl sulfide (EMS) (FIG. 2A-B). Analogous to MT-EtOH (10), use of DMS or EMS resulted in methane or ethane production, respectively, in a 1 to 1 stoichiometry (FIG. 3A-B). Specific deletion of the other two NFL genes, Rru_A0772-Rru_A0773, did not affect growth or hydrocarbon production (FIG. 2A-B and FIG. 8B). Thus, we designate R. rubrum genes Rru_A0793-Rru_A0796, previously identified as NFL genes nflBHDK of unknown function (8, 9), as methylthio-alkane reductase genes, marBHDK. This is based on corresponding amino acid similarity to R. rubrum molybdenum nitrogenase gene products NifB (synthesis of the NifB-cofactor precursor to the nitrogenase catalytic cofactor), NifH (nitrogenase-reductase iron protein), NifD (nitrogenase catalytic subunit α), and NifK (nitrogenase catalytic subunit β) (FIG. 10-FIG. 13). NFL genes Rru_A0772-Rru_A0773 remain designated nflDK genes of unknown function (8, 9).

When all R. rubrum NFL genes were deleted (strain Δ0772:3/Δ0793:6) and specific gene combinations were re-introduced via expression from a plasmid, expression of marBHDK was necessary and sufficient to restore growth and hydrocarbon metabolism from VOSCs (FIG. 2B-C and FIG. 9B-C). The NFL genes of unknown function, nflDK, could not replace marDK in complementing for growth. Upon feeding cells expressing marBH and nflDK with VOSCs, ethylene and ethane production was poorly catalyzed at 3- to 4-fold above basal levels and no methane enhancement was observed (FIG. 2B-C and FIG. 9B-C). This revealed that R. rubrum NflDK could only weakly catalyze methylthio-alkane reduction, indicating a different primary function. Given nflDK is expressed not just in the presence of MT-EtOH but also in response to general sulfate limitation (FIG. 1B-C), NflDK may catalyze sulfur liberation from alternate albeit unknown compounds. Alternately, given gene proximity and amino acid similarity (40%) to MarDK, NflDK may serve as accessory proteins for MarDK assembly analogous to NifEN (14). NifEN arose evolutionarily by gene duplication of NifDK and contains considerable sequence homology (˜40%) to NifDK, including P-cluster and FeMo-cofactor coordination sites (8, 9, 12). While NifEN does not have nitrogenase and hydrogen formation activity, it still retains acetylene and azide reduction capabilities (66). The R. rubrum NflDK, group IV nitrogenase-like proteins of unknown function (Rru_A0772-Rru_A0773 gene products) share 40% sequence identity with MarDK and are evolutionarily closer to MarDK than NifDK (FIG. 4). Coordinately, the nfIDK genes are located near marBHDK analogous to the association of nifEN with nigBHDK (8,9). However, unlike NifEN, NflDK is entirely dispensable, and homologous nflDK sequences are not observed to be present and associated with marBRDK gene clusters in several other organisms (FIG. 18).

These results demonstrated the requirement of the MarBHDK nitrogenase-like system for the anaerobic assimilation of sulfur from common environmental VOSCs such as DMS and MT-EtOH in order to support growth and methionine metabolism. Moreover, these observations revealed a previously unknown mechanism for the bacterial production of methane and ethylene.

### Methylthio-Alkane Reduetase Relleases Methanethiol From VOSCs for Methionine Biosynthesis

The link between VOSC utilization and methionine synthesis via the marBHDK gene products was characterized by feeding experiments with (2-[methyl-C¹⁴]thio)ethanol. This enabled detection of the methylthio-moiety of MT-EtOH. Upon feeding the wild type strain, MT-EtOH was consumed. Labeled methanethiol (C¹⁴H₃—SH) and methionine (methyl-C¹⁴) were concomitantly produced and observed at low levels (˜2% of MT-ETOH concentration) until MT-EtOH was depleted (FIG. 2D). These low levels, like previously observed for methanethiol metabolism from 5′-methylthioadenosine in R. rubrum (12), are likely due to the flux of methanethiol to methionine and subsequent utilization thereof for protein synthesis and SAM-dependent processes (11). This is substantiated by C¹⁴ incorporation from MT-EtOH into insoluble cell material (FIGS. 14A-14B). Conversely, in the marBHDK deletion strain there was no detectable metabolism of MT-EtOH, and hence, no methanethiol or methionine produced (FIG. 2E and FIGS. 14A-14B). Given that ethylene, ethane, and methane are produced from MT-EtOH, EMS, and DMS, respectively, the observed methanethiol is consistent with C—S single bond reduction and methylthio-release from these substrates by the methylthio-alkane reductase (FIG. 1A, reaction 1 and FIG. 2F). Each process is thermodynamically favored for the substrates and products observed (FIG. 2F and FIGS. 15A-15B). The methanethiol along with O-acetyl-homoserine then serve as substrates for O-acetylhomoserine sulfhydrylase, which catalyzes the synthesis of methionine (FIG. 1A, reaction 3) (13). This defines an anaerobic methylthio-alkane reductase methionine synthesis pathway and establishes the role of a nitrogenase-like enzyme system in sulfur metabolism (FIG. 1A).

### Native Expression of Methylthio-Alkane Reductase is Regulated by Sulfur Response

SalR—Sulfur metabolism evidently is the primary function of these nitrogenase-like methylthio-alkane reductases, as opposed to nitrogen fixation by nitrogenase. R. rubrum possesses molybdenum nitrogenase (NifHDK), which is the default nitrogenase, and iron only nitrogenase (AnfHDGK) nitrogenase, which is synthesized in the absence of molybdenum (9). In in vivo activity assays, the R. rubrum molybdenum nitrogenase could not perform methylthio-alkane reduction, even under maximally inducing conditions, and vice versa (FIG. 3D; glutamate as N-source and 50 μM sulfate). Indeed, nitrogenase and methylthio-alkane reductase activities were independent, separately regulated, and both systems could be expressed simultaneously (FIG. 3D). R. rubrum nitrogenase gene expression (nifHDK) is regulated by the transcriptional regulator NifA in response to nitrogen availability (14). Methylthio-alkane reductase activity in the presence of 1 mM MT-EtOH or DMS was regulated by sulfate availability, with an EC₅₀˜150 μM sulfate for 50% repression of activity (FIG. 3C). Our random mutagenesis screen identified the specific regulatory gene in the vicinity of marBHDK (Rru_A0785; FIG. 1B, FIG. 6, and FIGS. 7A-7D). We designate this LysR family regulator as SalR (sulfur salvage regulator). Inactivation of salR rendered strains incapable of growth or hydrocarbon production utilizing MT-EtOH, DMS and EMS as sole S-source (FIG. 2A-B and FIG. 8E; strain 0785::Tn5). Transcriptomics and differential expression analysis of the parent (WRdht) and salR deletion strain (0785::Tn5) growing under marBHDK inducing and repressing conditions revealed that marBHDK and the rest of the methylthio-alkane reductase methionine synthesis pathway are under transcriptional control of SalR (FIG. 1C). Thus, when sufficient sulfur is available (>150 μM), expression appears repressed, but when sulfate becomes limiting, marBHDK and O-acetylhomoserine sulfhydrylase gene transcription is specifically upregulated via SalR to utilize VOSCs for methionine metabolism (FIG. 1A; reactions 1 and 3). Therefore, as shown in FIG. 2B, expression of marBHDK from a non-natural gene promoter DNA sequence enables synthesis of MarBHDK and concomitant ethylenelethanelmethane production without the native regulation imposed by sulfate-sensitive SalR.

### Organisms With Methylthio-Alkane Reductase are Widespread in Nature Including Industrially Relevant Acetogenic and Lignocellulosic Clostridia

The nitrogenase superfamily is composed of the bona fide nitrogenase sequences (groups I-III) and nitrogen fixation-like sequences (NFL; groups IV-VI) (FIG. 4) (9). Phylogenetic analysis places methylthio-alkane reductase homologues in their own clade within group IV, which we denote as group IVC (FIG. 4 and FIG. 16). In contrast, the R. rubrum NflD protein resides in a separate clade with other NflD sequences of unknown function (FIG. 4), consistent with the poor methylthio-alkane reductase activity exhibited by NflDK (FIG. 2B). Bacteria possessing MarBHDK sequence homologs of this previously uncharacterized group IV-C clade include members of the Fibrobacter and Bacteriodetes phyla, Rhodospirillales and Rhizobiales within the Proteobacteria phylum, and Selenomonadales and Clostridium species within the Firmicutes phylum (FIG. 17). To verify the phylogeny results for the Proteobacteria, Rhodopseudomonas palustris and Blastochloris viridis were tested, which possess group IV-C marBHDK homologues. Also tested was closely related species Rhodobacter capsulatus, which possesses nitrogenase and nflBHDK but no marBHDK (FIG. 4, FIG. 16, and FIG. 18; Rp, Bv, Rc). Both R. palustris and B. viridis were able to grow with MT-EtOH, EMS, or DMS as sole sulfur source and correspondingly produced ethylene, ethane, or methane (FIG. 2A and FIGS. 19A-19C), demonstrating that methylthio-alkane reductase homologues from these organisms catalyze the same process. Conversely, R. capsulatus could not utilize any of these VOSCs as sole sulfur source for growth (FIG. 2A and FIGS. 19A-19C), like R. rubrum expressing NflDK but not MarDK (FIGS. 2B-C), indicating that group IV NFL proteins of unknown function catalyze processes distinct from methylthio-alkane reductase.

### Amino Acid Sequence Comparison of Nitrogenase and Methylthio-Alkane Reductase Proteins Indicate a Distinct Function for Each Group

Nitrogenase functions via a coordinated transfer of electrons through a network of highly modified iron and sulfur metal clusters. The minimal molybdenum nitrogenase system requires gene products NifBHDKEN; the vanadium (Vnf) and iron (Anf) nitrogenases have similar requirements (8, 9). The NifH homodimer possesses a single Fe₄—S₄ cluster at the homodimer interface. The NifDK heterotetramer contains Fe₈—S₇ P-clusters coordinated at each of the two NifDK subunit interfaces, and each NifD subunit contains the characteristic catalytic FeMo-cofactor [Fe₇—S₉—C—Mo-homocitrate] (12). In the Vnf and Anf nitrogenase systems Mo is replaced with V or Fe, respectively. Initially, electrons are donated to the NifH Fe₄—S₄ cluster from a reducing agent such as a ferredoxin or flavodoxin (61). When NifH is in complex with NifDK, these electrons are transferred in an ATP binding and hydrolysis dependent manner to the P-cluster of NifDK. NifH also has roles in P-duster assembly from two Fe₄—S₄ clusters on the apo-NifDK heterotetramer and synthesis of FeMo-cofactor when in complex with NifDK-like FeMo-cofactor assembly proteins, NifEN (12). P-cluster electrons are then passed to the FeMo-cofactor catalytic cluster and ultimately to FeMo-cofactor-bound dinitrogen for stepwise reduction to ammonia (17, 62).

MarH: MarH contains the same NifH conserved residues for MgATP hydrolysis and Fe₄—S₄ cluster coordination that enables transfer of electrons from the NifH Fe₄—S₄ cluster to the NifDK P-cluster (FIG. 12). The NifH conserved Arg-100 (V. vinelandii numbering) is also conserved in MarH. This residue is modifiable by ADP-ribosylation to prevent NifH from complexing from NifDK. As nitrogenase activity is an ATP intensive process, this post translational modification effectively inactivates nitrogenase to prevent unnecessary ATP consumption when energy supply is insufficient or diazotrophy is not required (e.g. ammonium available as N-source). For R. rubrum nitrogenase, ADP-ribosylation is catalyzed by dinitrogenase reductase ADP-ribosyltransferase (DRAT) and removed by dinitrogenase reductase activating glycohydrolase (DRAG). An analogous system appears to exist in A. vinelandii (63).

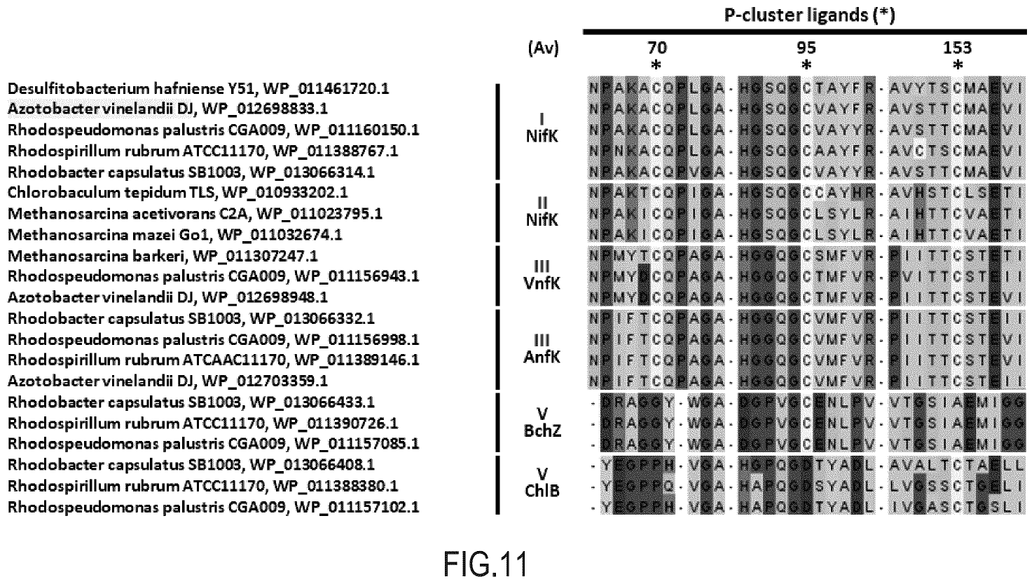

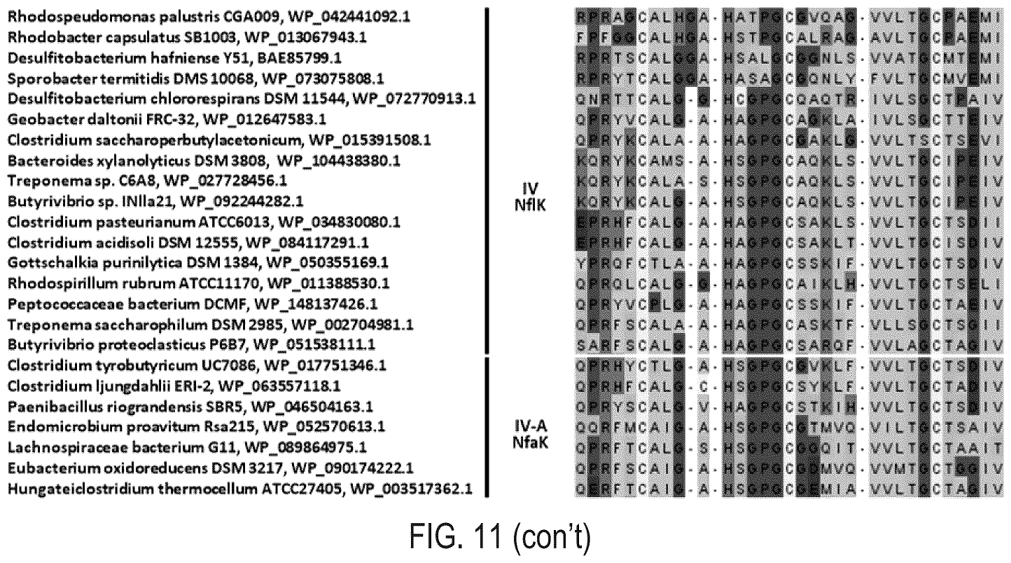

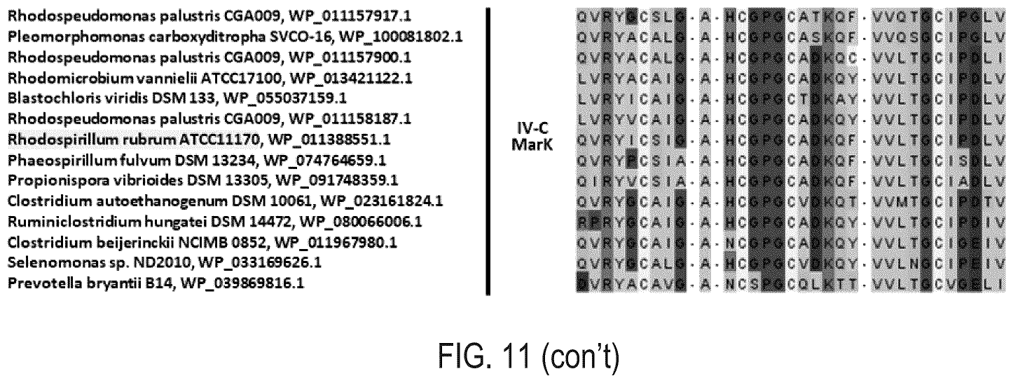

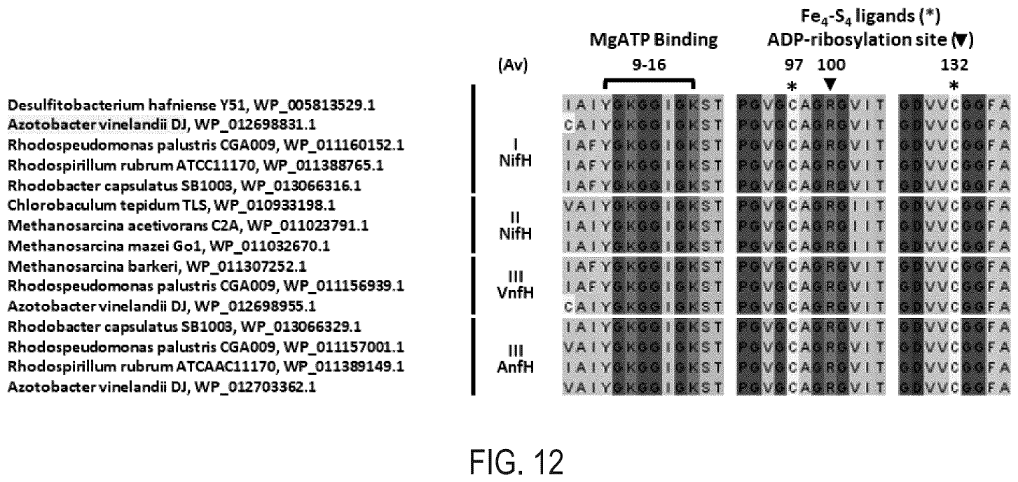

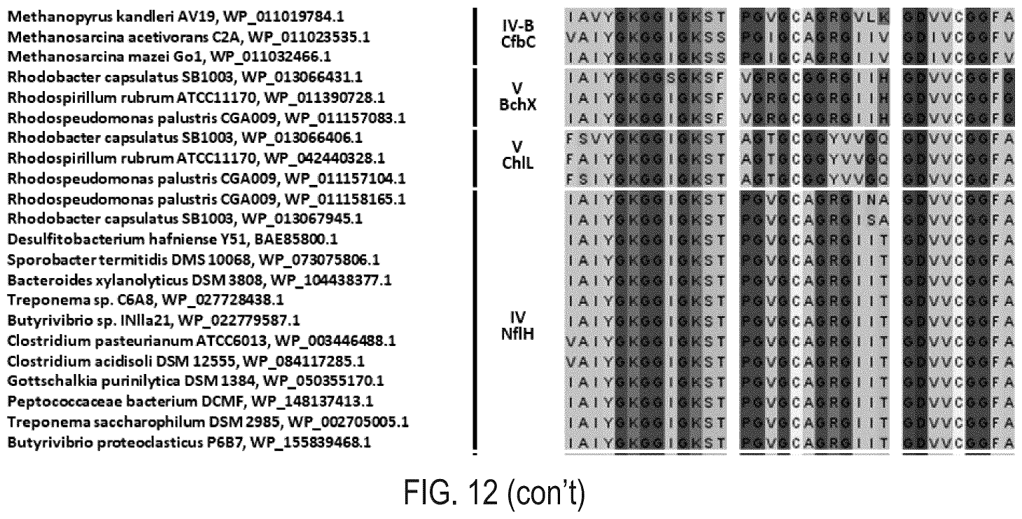

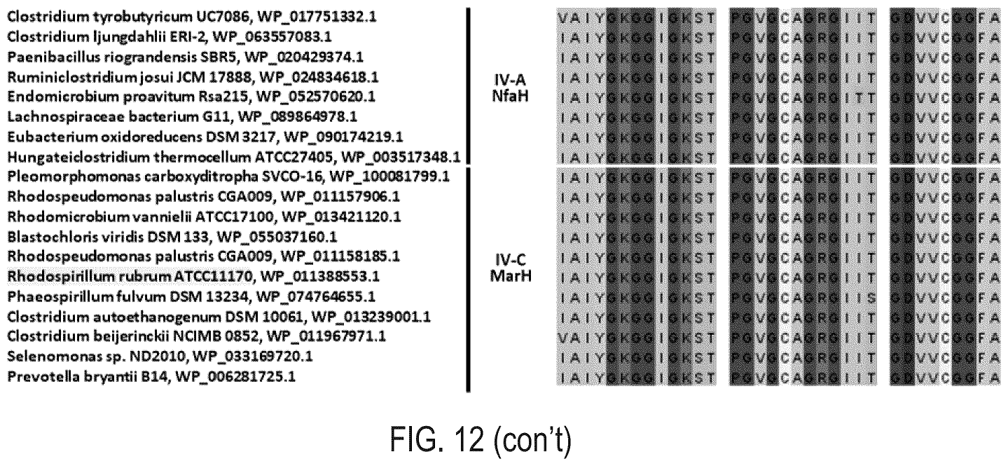

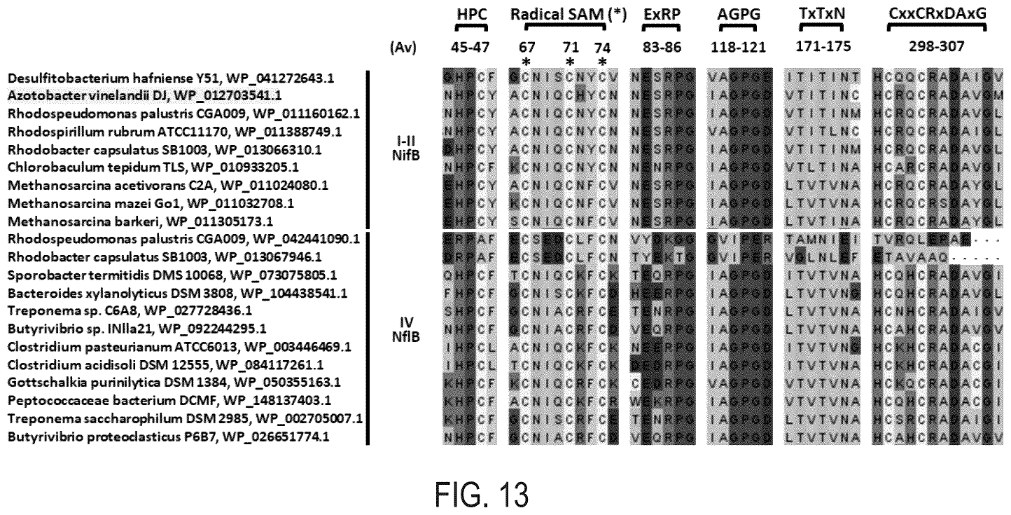

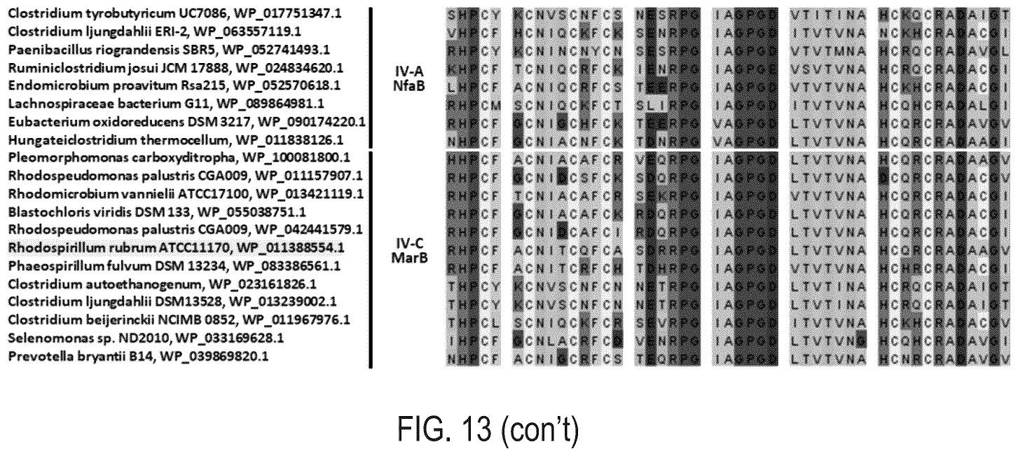

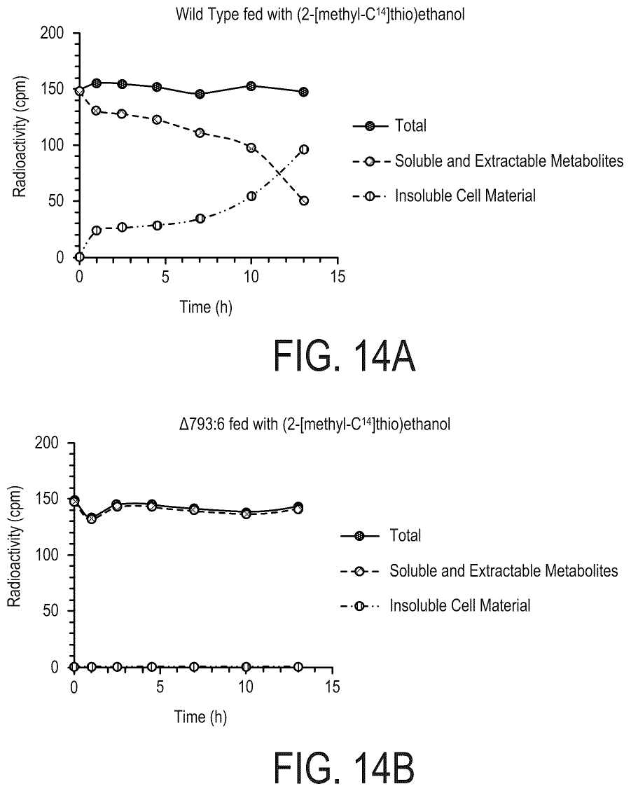

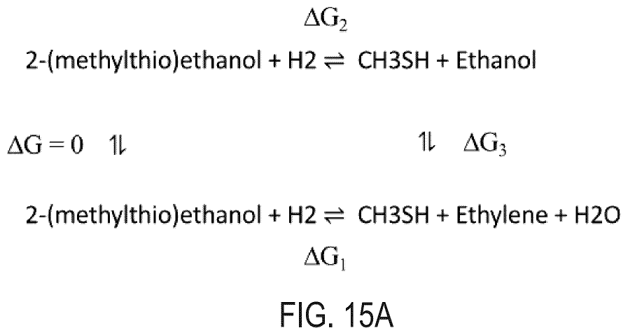

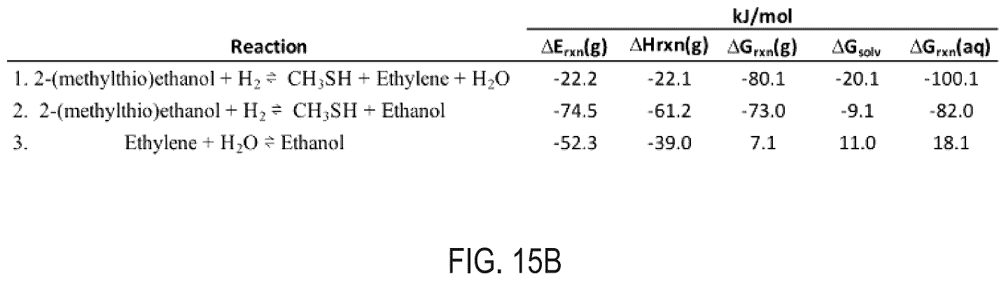

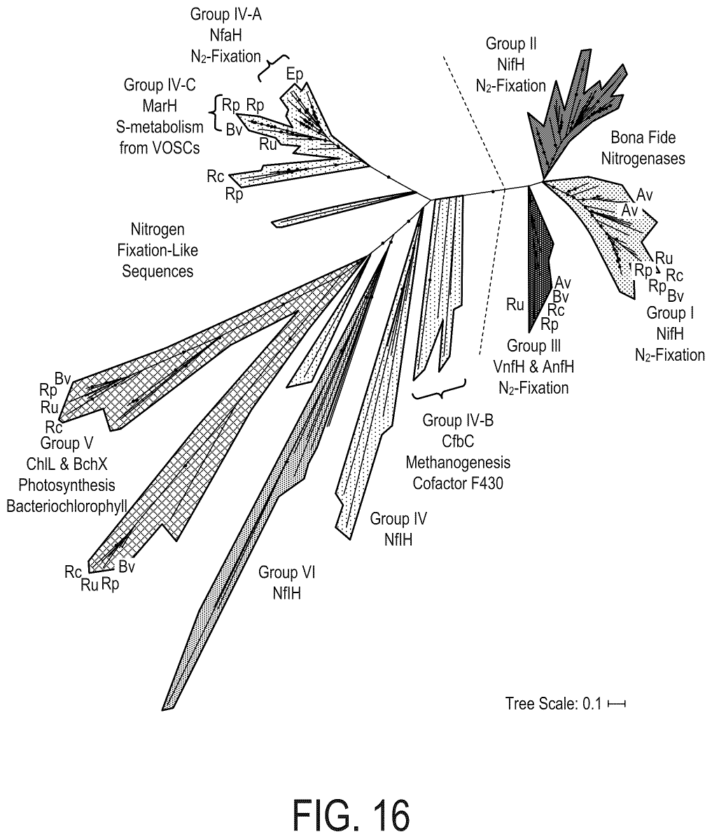

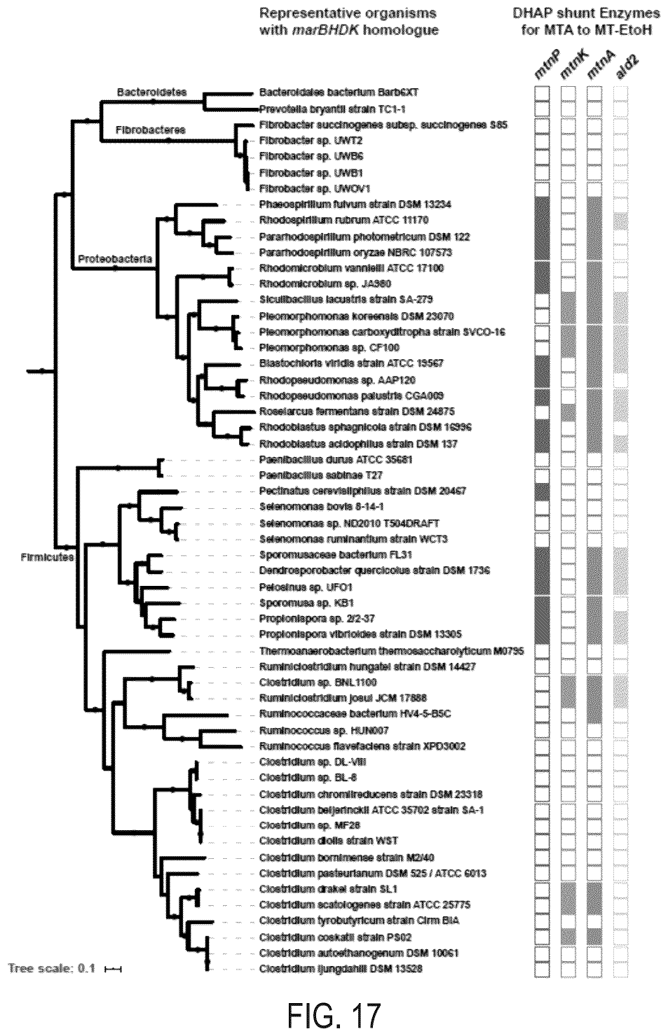

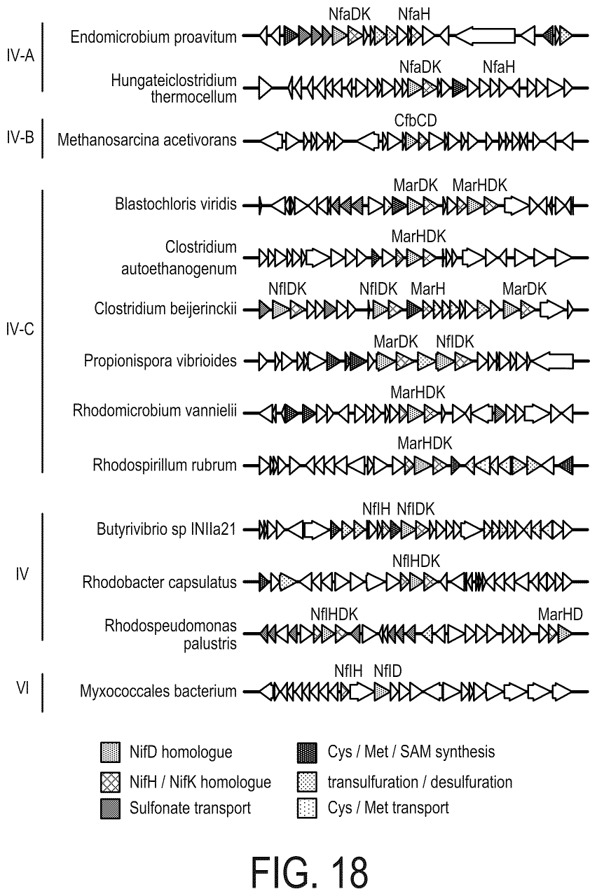

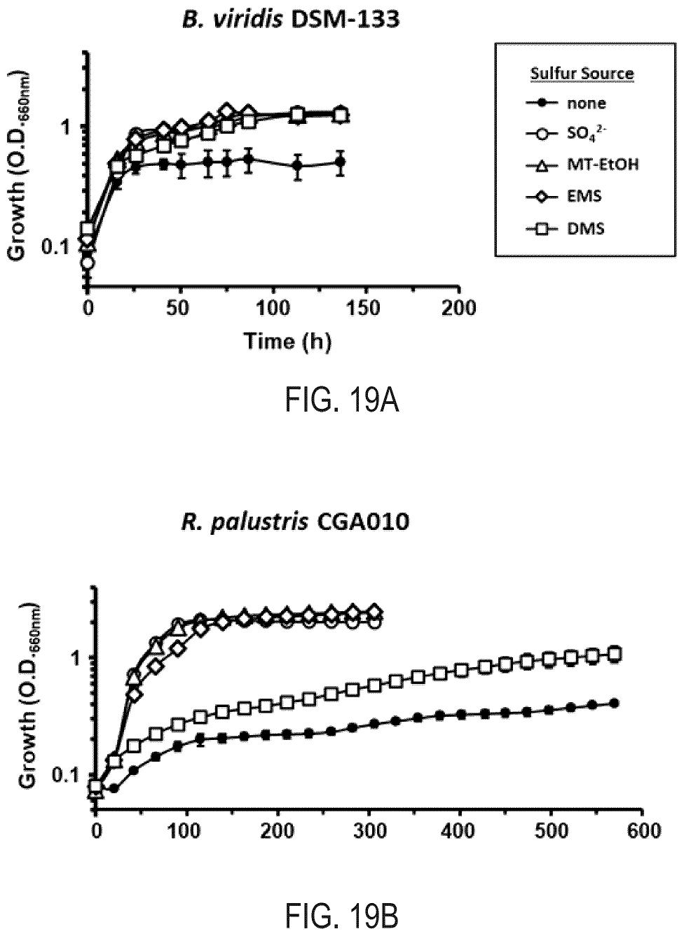

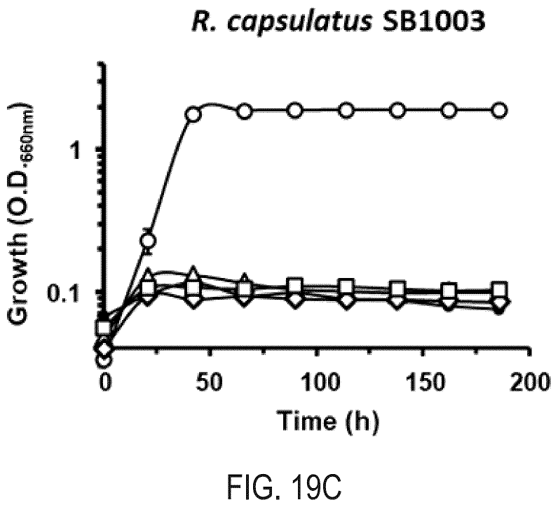

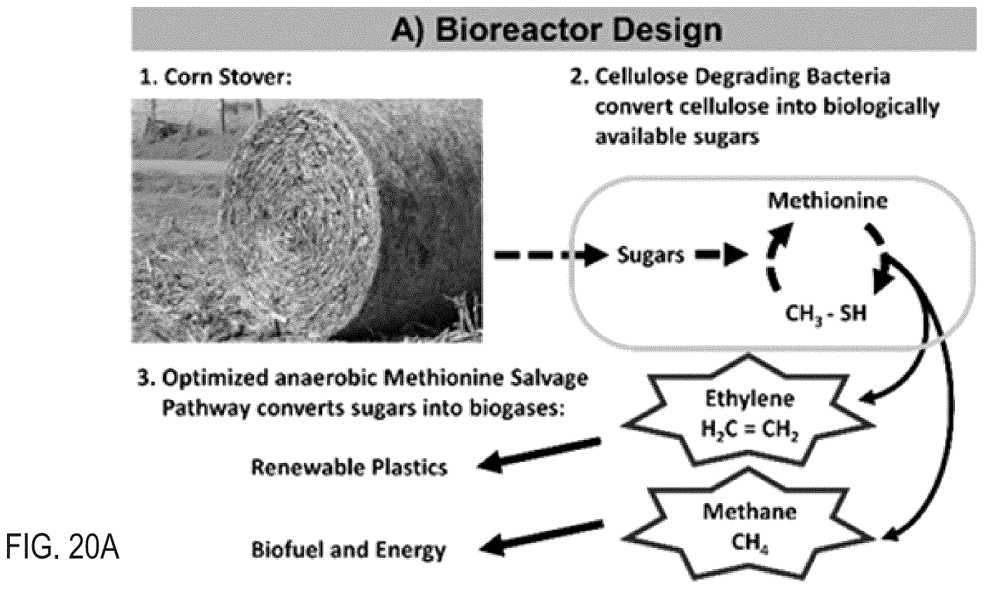

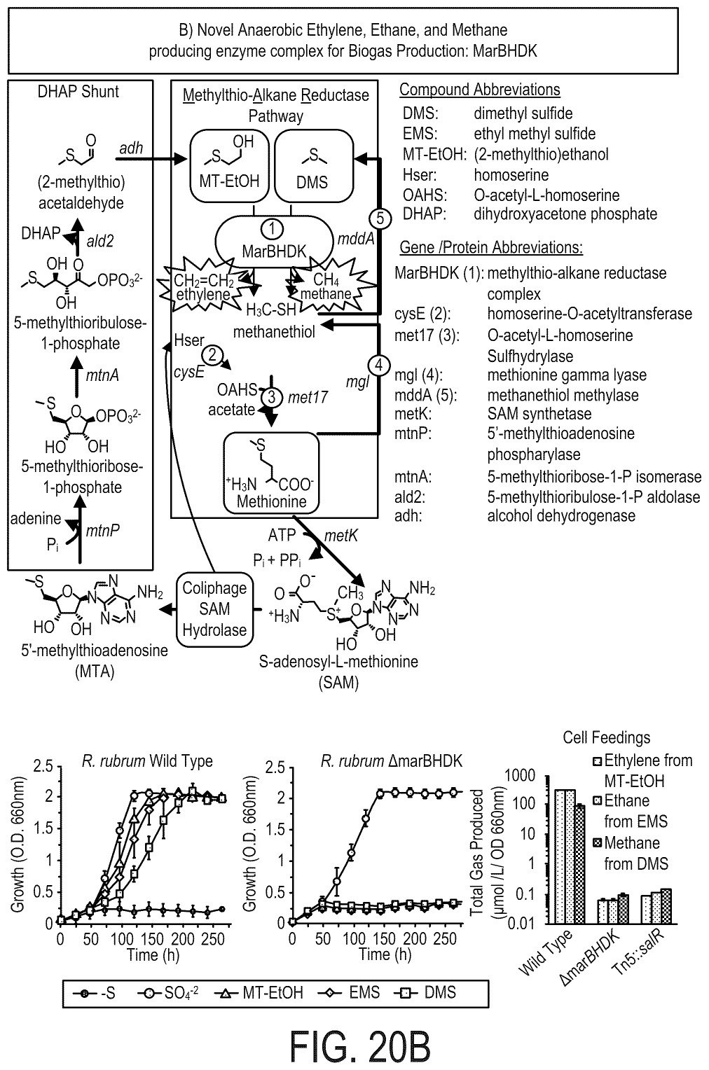

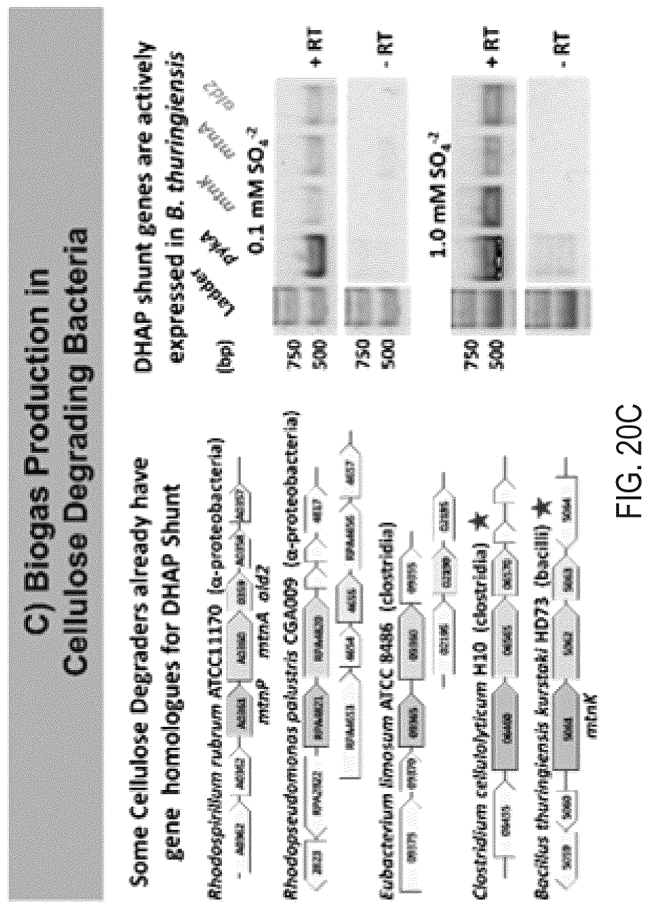

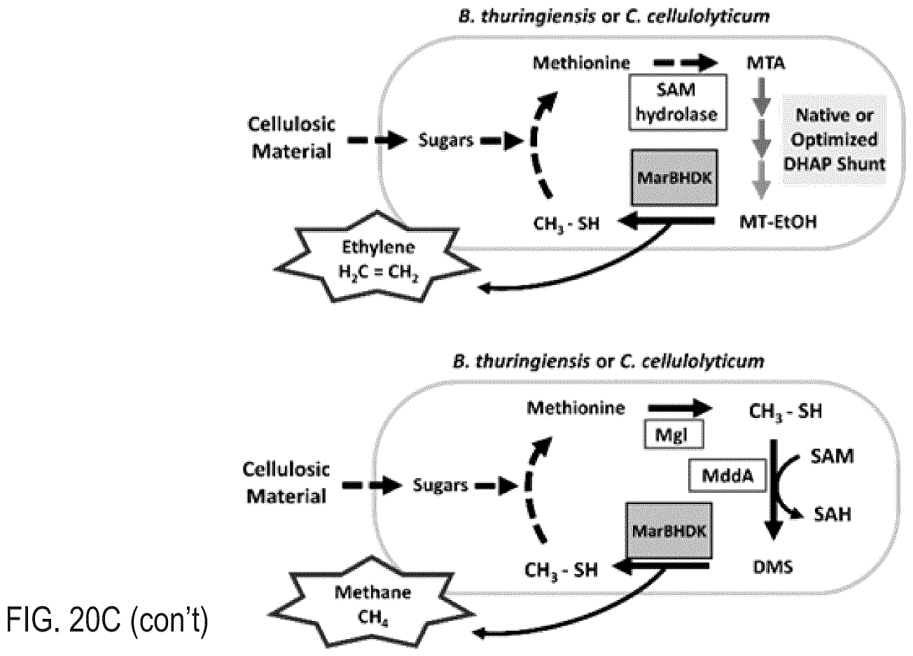

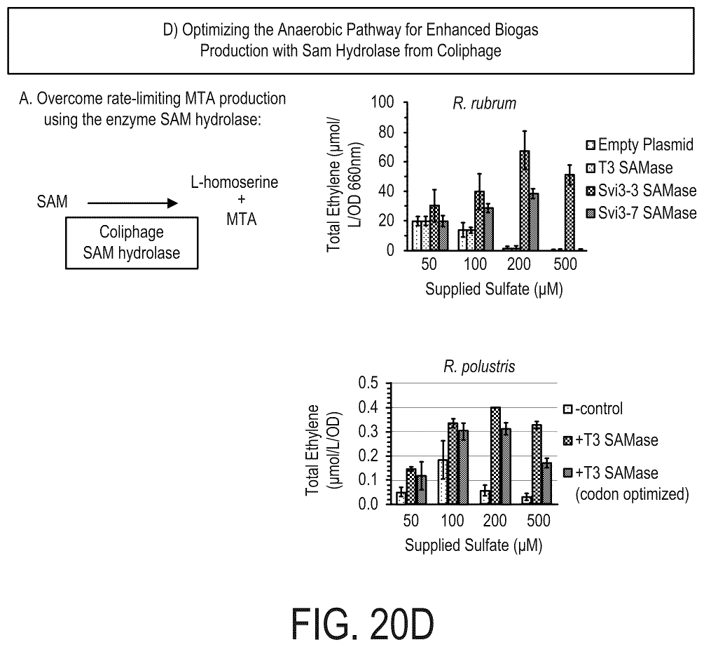

MarDK: MarD and MarK each possess the triad of cysteines conserved in the molybdenum nitrogenase subunits NifD and NifK for P-cluster coordination (FIG. 10 and FIG. 11). One or more of these conserved cysteines are absent in the bacteriochlorophyll oxidoreductase (ChlLNB and BchXYZ) and reductive cyclase F430 synthesis (GbfCD) systems, which complex a catalytic Fe₄—S₄ cluster instead (64, 65). MarD also has a conserved cysteine for coordinating a catalytic metallocofactor as in NifD for the FeMo-cofactor (Cys-275 in A. vinelandii). In contrast, however, the conserved NifD His-442 residue (A. vinelandii numbering) responsible for coordinating FeMo-cofactor homocitrate and molybdenum is replaced with a Gly-Asp-Glu motif in MarD and there are no homocitrate synthase genes associated with marBHDK gene clusters (FIG. 10) (9, 15, 16). In addition, the conserved NifD Glu-191 and His-195 residues involved in coordinating nitrogen intermediates bound to the FeMo-cofactor are replaced in MarD with aromatic residues Trp and Phe (9, 17).

MarB: NifB is a radical SAM enzyme responsible for carbide insertion and formation of the 8Fe—9S—C NifB-cofactor, the precursor to FeMo-cofactor (12). MarB possesses all of the identified motifs conserved across NifB enzymes associated with bona fide nitrogenases (FIG. 13). For nitrogenase, NifB-cofactor maturation to FeMo-cofactor requires NifH and NifEN for addition of molybdenum and homocitrate (12).

Together, this indicates that methylthio-alkane reductase proceeds via a mechanism, similar but distinct to that of nitrogenase to convert MT-EtOH to ethylene, ethylmethylsulfide to ethane, and dimethylsulfide to methane (17). Methane release from DMS by the methylthio-alkane reductases is separate and distinct from the other known non-archaeal methanogenic processes, including photosynthesis-linked methane production by cyanobacteria (18), methane release from methylphosphonates by marine bacteria (19), and direct reduction of carbon dioxide to methane by iron-only nitrogenase (AnfDHGK) (20). In waterlogged soils, strictly anaerobic microbial processes produce ethylene that can accumulate to levels inhibitory to plant root growth, causing crop damage (21, 22). Early attempts at identifying ethylene-producing organisms surprisingly isolated oxygen-dependent soil bacteria and fungi (23, 24). The organisms and methylthio-alkane reductases identified here function a,naerobically and could contribute to this soil-ethylene paradox (10). This anaerobic ethylene process is distinct from the oxygen-dependent reactions catalyzed by aminocyclopropanecarboxylate oxidase and 2-oxoglutate dioxygenase in plants, fungi, and certain bacteria.

### Non-Natural Pathways for Optimized Microbial Ethylene and Methane Production

The ethylene precursor, 5′-methylthioadenosine (MTA) is a routine byproduct of highly regulated processes such as quorum sensing, polyamine production, etc. These are highly regulated processes, making the native production of MTA for subsequent ethylene production rate limiting. The coliphage SAM hydrolase (MTA-forming) is a viral enzyme that directly converts SAM to MTA (FIG. 20D) (69, 70). When this non-naturally occurring gene element is synthesized in Rhodospirillum rubruin and Rhodopseudomonas palustris for ethylene biogas production vial the DHAP shunt MarBHDK system (FIG. 20C), ethylene production is enhanced 20-50 fold above the native amount produced by the organism in the absence of SAM hydrolase (FIG. 20D).

The methane precursor, dimethylsulfide, is the most abundant organic sulfur compound in the environment. It is produced by marine bacteria from dimethylsulfinypropionate and by terrestrial bacteria from methanethiol (71, 72). A non-natural methionine salvage pathway from Rhodopseudomonal palsutris for the conversion of methionine to dimethylsulfide is constructed using methionine gamma lyase (mgl) and methanethiol methyltransferase (mddA) (FIG. 20B) (72). This directly converts methionine to dimethylsulfide for methane production by methylthio-alkane reductase (MarBHDK) (FIG. 20C) in photosynthetic bacteria (e.g. Rhodospirillum rubrum) or lignocellulose degrading bacteria (e.g. Clostridium cellulolyticum).

### Materials and Methods

Fine chemicals: Dimethyl sulfide, methanethiol, L-methionine, 5′-methylthioadenosine, and S-methyl-t-cysteine were from Sigma; ethyl methyl sulfide, (2-methylthio)ethanol, (2-methylthio)acetate, and (3-methylthio)propanol were from Alfa Aesar. All media components were of ultrapure grade from Sigma or J. T. Baker, For targeted metabolite detection, (2-[methyl-C¹⁴]thio)ethanol was synthesized from [methyl-C¹⁴]-S-adenosylmethionine (Perkin Elmer). Labeled S-adenosylmethionine was acid hydrolyzed in 0.01 N H₂SO₄ under reflux at 100° C. for 30 min to form [methyl-C¹⁴]-5′-methylthioadenosine. (2-[methyl-C¹⁴]thio)ethanol was subsequently formed enzymatically in a reaction containing 50 mM potassium phosphate pH 7.8, 5 mM MgCl₂, 0.2 mM NADH, 60 μM substrate, and 2 μM each of purified R. rubrum 5′-methylthioadenosine phosphorylase (10), Bacillus subtilis 5-methylthioribose-1-phosphate isomerase (29), E. coli 5-methylthioribulose-1-phosphate aldolase (25), and S. cerevisiae alcohol dehydrogenase (Sigma) at 30° C. for 2 h. Enzymes were synthesized and purified as previously described (10). Complete conversion was monitored by reverse phase HPLC with an inline scintillation detector as previously described (10), followed by enzyme removal via Amicon (Millipore) centrifugal concentration device.

Bacterial strains and growth conditions: R. rubrum ATCC 11170 wild type strain (Sm^{R}; NC_007643.1; American Type Culture Collection), Rru_A1998 deletion strain WR (ΔrlpA::Gm^{R}) in which the MTA-isoprenoid shunt is inactivated, and Rru_A1998/Rru_A0359 deletion strain WRdht (ΔrlpA::Gm^{R}/Δald2) in which the MTA-isoprenoid and DHAP shunts are inactivated were as previously described (10, 30). Rhodobacter capsulatus SB1003 (NC_014034.1, American Type Culture Collection) (31), Rhodopseudomonas palustris CGA010 (32), and Blastochloris viridis DSM133 (NZ_AP014854.2, University of Leibnitz DSMZ) (33) wild type strains were also as previously described. Rhodopseudomonal palustris CGA010 (Caroline Harwood, University of Washington) is a derivative of CGA009 (Sm^{R}; NC_005296.1, American Type Culture Collection) in which a frame shift mutation is corrected. Anaerobic growth of R. rubrum and R. capsulatus was performed in static anaerobic culture tubes and serum bottles at 30° C. with 2000 lux incandescent illumination. Cultures were composed of sulfur-free Ormerod's malate (30 mM) minimal medium supplemented with the indicated sulfur source under a 95:5 mixture of N₂LH₂ gaseous headspace as previously described (34, 35). Anaerobic growth of R. palustris was similarly performed by replacing malate with 0.5% (v/v) ethanol and 0.2% (w/v) sodium bicarbonate and adding 2 μg/ml para-aminobenzoic acid. All anaerobic manipulations were performed using an anaerobic chamber under 5% hydrogen and 95% nitrogen (Coy Laboratories).

Anaerobic growth of B. viridis was performed in anaerobic cultures tubes continuously rotated on a rotisserie at 30° C. with 2000 lux incandescent illumination. Cultures were composed of a modified sulfur-free succinate medium 27 (N medium) (36) supplemented with the indicated sulfur source under an N₂ gaseous headspace. Briefly, sulfur-free succinate medium 27 contained (per liter water) 0.3 g yeast extract, 1.0 g Na₂-succinate, 0.5 g ammonium acetate, 5 mg Fe(III) citrate, 0.5 g KH₂PO₄, 0.33 g MgCl₂.6H₂O, 0.4 g NaCl, 0.4 g NH₄Cl, 0.05 g CaCl₂.2H₂O, 0.4 ml of 0.1 g/L vitamin B12 solution, 0.5 ml of 1.0 g/L resazurin solution, and 1.0 ml of trace element solution [(per liter water) 0.075 g Zn-acetate, 0.03 g MnCl₂.4H₂O, 0.3 g H₃BO₃, 0.20 g CoCl₂.6H₂O, 0.01 g CuCl₂.2H₂O, 0.02 g NiCl₂.6H₂O, 0.03 g Na₂MoO₄.2H₂O] at pH 6.8. Media was brought to a boil, dispensed and sealed in anaerobic culture tubes, sparged with N₂ until anaerobic, autoclaved, cooled, supplemented with the appropriate sulfur source, and reduced with Tris-buffered titanium citrate pH 8.0 (1 mM final concentration) before inoculating.

Proteomics analysis: To optimize ethylene induction, and by inference of the remaining steps of the pathway in metabolizing MT-EtOH to methionine, the growth of R. rubrum strain WR (ΔrlpA::Gm^{R}) was measured spectrophotometrically by optical density at 660 nm (O.D.₆₆₀ₙₘ) and the specific rate of ethylene production (μmol/h/g dry cell weight) was independently measured by gas chromatography (see GC analysis below) at regular intervals for a given sulfate or MT-EtOH concentration (FIGS. 5A-5C). Cells were grown anaerobically, photoheterotrophically in anaerobic culture tubes containing 20 ml of sulfur-free malate minimal medium supplemented with 25, 50, 100, 1000 μM ammonium sulfate or 200-1000 μM MT-EtOH. For limiting sulfate, maximum ethylene specific rate was observed under 50 μM sulfate at an O.D.₆₆₀ₙₘ of 0.6-0.75. For 200-1000 μM MT-EtOH, maximum ethylene specific rate was also observed in the same O.D.₆₆₀ₙₘ range. Subsequently, R. rubrum strain WR was grown in triplicate (biological replicates) anaerobically, photoheterotrophically in rectangular flasks containing 0.5 L sulfur-free malate minimal medium supplemented with 50 μM or 1000 μM ammonium sulfate or 1000 μM MT-EtOH to an O.D.₆₆₀ₙₘ of ˜0.60. Cultures were harvested anaerobically by centrifugation at 3000×g for 5 min and remaining media was thoroughly removed by decanting. Cell pellets were aliquoted in 0.4-0.6 g fractions and flash frozen in liquid N₂.

Each cell pellet was lysed by 4% sodium deoxycholate in 100 mM ammonium bicarbonate with the application of sonication (20% amplitude, 10 s pulse, 10 s rest, 2 min total puke time). Crude protein extract was precleared via centrifugation, reduced with 10 mM dithiothreitol, alkylated with 30 mM iodoacetamide, and then collected on top of a 10 kDa cutoff spin column filter (VIVASPIN 500, Sartorius). Collected proteins were digested to peptides with two sequential aliquots of sequencing-grade trypsin (Sigma) at a 1:75 enzyme:protein ratio (w/w), initially overnight at room temperature followed by additional 3 h at room temperature. Peptides were collected by centrifugation and acidified to 1% formic acid followed by extraction with ethyl acetate to remove sodium deoxycholate. The peptide containing aqueous phase was recovered and concentrated. Concentrated peptides were measured using the bicinchoninic acid assay (Pierce).

Each peptide mixture was analyzed on a two-dimensional liquid chromatography tandem mass spectrometry (2D-LC-MS/MS) platform using a Q Exactive Plus (QE+) mass spectrometer (Thermo Fisher Scientific) equipped with an Ultimate 3000 RS system (Thermo Fisher Scientific). 9 μg of each peptide sample was loaded via autosampler onto a triphasic pre-column (5 cm C18 reversed phase (RP), 5 cm strong cation exchange, and 5 cm C18 RP). Bound peptides were then washed and separated over three successive salt cuts of ammonium acetate (35 mM, 50 mM and 500 mM), each followed by an RP-LC elution via an in-house pulled nano-electrospray emitter (75 μm ID) packed with 30 cm of C18 RP. Mass spectra were acquired on QE+ in a data-dependent mode with full scan at 70K resolution, followed by HCD fragmentation of the top 15 most abundant ions at 15K resolution.

Acquired MS/MS spectra were matched with theoretical tryptic peptides generated from a concatenated Rhodaspirillum rubrum proteome FASTA database with contaminants and decoy sequences using MyriMatch v. 2.2 (37). Peptide spectral matches were filtered to achieve peptide false-discovery rates (FDR) <1% and assembled to their respective proteins using IDPicker v. 3.1 (38). Peptide abundance intensities were derived in IDPicker by extracting precursor intensities from chromatograms with lower and upper retention time of 90 s and tnass tolerance of 5 ppm. Protein abundances were calculated by summing up intensities of all identified peptides and normalized by their protein lengths respectively. Protein intensities were further log2 transformed and median centered using InfernoRDN version 1.1 (39), to approximate a normal distribution and reduce technical variance for further pairwise comparison. Student's T-test was then performed for every pair condition using Perseus platform (40) for two different thresholds (Benjamini-Hochberg FDR adjusted p-value <0.05 and fold change >2, or Benjamini-Hochberg FDR adjusted p-value <0.01 and fold change >4; two-sided).

Transcriptomies analysis: R. rubrum strain WRdht (ΔrlpA/Δald2) and 0785::Tn5 (ΔrlpA/Δald2/0785::Tn5) were grown in triplicate (biological replicates) photoheterotrophically in anaerobic culture tubes containing 20 ml sulfur-free malate minimal medium supplemented with 50 μM (“Lo”) or 1000 μM (“Hi”) sulfate. When cells reached an O.D.₆₆₀ₙₘ of 0.65-0.8, cells were harvested and stabilized by RNA protect reagent (Qiagen). RNA was isolated using the RNeasy protect kit (Qiagen) and quantified by UV absorbance. RNA-seq library construction and sequencing were performed at The Genomics and Microarray Shared Resource at University of Colorado Denver Cancer Center, Denver, CO, USA. Library preparation and rRNA depletion were performed using to the Zymo-Seq Ribo Free Total RNA Library Kit Cat No. R3000 with input of 250 ng and libraries were sequenced on the Illumina NovaSeq 6000 using 2×150 paired end reads. Raw RNA-seq data were trimmed using sickle (github.com/najoshi/sickle) (41). Prior genomic sequencing of R. rubrum strain WRdht confirmed the rlpA and ald2 deletions and >99% nucleotide identity to the R. rubrum ATCC11170 genome. Mapping of transcriptomic reads to the reference was conducted using Bowtie2 (v2.3.5.1) with the options—very-sensitive and—score-min L,0, −0.1 (42). Differential expression analysis was performed using DESEq2 (v 1.22.2) (fitType=local, test=Wald) (43). Comparison of transcriptomes from the parent strain (WRdht) grown under 50 μM versus 1000 μM sulfate indicated all genes that were transcriptionally regulated >1.5-fold in response to sulfate availability (two-sided Wald Chi-square test, BH-FDR adjusted p<0.002 as implemented by DESeq2 (43)). Corresponding comparison for the SalR deletion strain (0785::Tn5) indicated which of these genes were no longer regulated in response to sulfate availability. Comparison of the SalR deletion strain to the parent strain under 1000 μM sulfate indicated which of these genes were potentially transcriptionally activated or repressed by SalR.

Transposon mullagenesis: R. rubrum strain WRdht (ΔrlpA::Gm^{R}/Δald2) was randomly mutagenized using the efficient mini-Tn5 transposable element (44). R. rubrum was initially grown aerobically at 30° C. to late log phase in PYE liquid medium (3 g/L peptone, 3 g/L yeast extract, 266 mg/L MgSO₄.7H₂O, 75 mg/L CaCl₂.2H₂O, 11.8 mg/L FeSO₄.7H₂O, 20 mg/L ethyl enediaminetetraacetic acid, 1 mlt/ Ormerod's trace elements solution (31),1 mg/L thiamine, 1 mg/L nicotinic acid, 15 μg/L biotin). Donor strain, E. coli BW20767/pRL27 (Coli Genetic Stock Center, Yale) (44), was grown in lysogeny broth at 37° C. to mid exponential phase. Strains were separately centrifuged and washed three times with PYE medium, combined in a 1:2 ratio of E. coli to A. rubrum, concentrated, and spotted onto a 16% PYE agar plate. Biparental conjugation was carried out aerobically at 30° C. in the dark for no more 24 h to ensure R. rubrum cells received no more than one Tn5 insertion per genome. R. rubrum transconjugants were selected on 16% PYE agar plates with 25 μg/ml kanamycin and 30 μg/ml gentamycin under the same growth conditions.

Transposon-insertion isolates of R. rubrum were individually picked into 96-well flat-bottom tissue culture plates containing 200 μl of sulfur-free Ormerod's malate minimal medium supplemented with 100 μM ammonium sulfate and 25 μg/ml kanamycin. Inoculated plates were incubated in an anaerobic chamber for 2 h, sealed with thermal adhesive film to prevent evaporation, and further sealed in thermal-seal bags (Kapak, ProAmpac) to maintain anaerobic conditions. Isolates were grown anaerobically at 30° C. under 2000 lux incandescent illumination to late log phase. Cultures were briefly exposed to air atmosphere, quickly transferred by 96-pin transfer device to new anaerobic 96-well plates containing 200 μl of anaerobic sulfur-free Ormerod's malate minimal medium supplemented with 1 mM ammonium sulfate or 1 mM MT-EtOH, and then incubated and sealed in an anaerobic chamber as before. Isolates were again grown anaerobically under illumination to screen for mutants incapable of growth on MT-EtOH but still able to grow on sulfate as sole S-source. 11,250 mutants were screened to ensure each gene received a transposon insertion at least once (FIGS. 7A-7D). Putative ethylene pathway mutants were verified by confirmatory growth experiment in anaerobic culture tubes. The false discovery rate was 80% due to the sensitive nature of growing R. rubrum in 96-well plates with MT-EtOH as sole S-source. Validated ethylene pathway mutants were sequenced to determine the location of the Tn5 insertion as previously described (44,).

Gene deletion and complementation studies: Nonpolar gene cluster deletions of Rru_A1066-Rru_A1069, Rru_A0772-Rru_A0773, and Rru_A0793-Rru_A0796 in the R. rubrum wild type strain were performed by homologous recombination using previously described methods (10). Briefly, DNA fragments were amplified by PCR using primers listed in Table A below, digested with the indicated restriction enzyme following manufacturer's protocols (New England Biolabs), and ligated into pK18mobSacBgm (10) using T4 DNA ligase (New England Biolabs). Sequence verified plasmids were transformed into E. coli Stellar strain (TaKaRa Bio) and mobilized into R. rubrum wild type by triparental conjugation with helper strain E. coli JM109/pRK2013 (American Type Culture Collection) (45), similar to methods used for the transposon mutagenesis. Transconjugants were selected on 16% PYE agar plates with 25 μg/ml kanamycin and 50 μg/ml streptomycin under aerobic growth at 30° C. First and second homologous recombination events were selected by 10% (w/v) sucrose sensitivity and kanamycin resistance of the isolates, and second recombinants possessing the proper gene deletion were sequence verified.

TABLE A
Primers and Plasmids Used

| Primer | Primer | Fragment | Sequence | Fragment | Fragment |
|---|---|---|---|---|---|
| Description | Name | Number | (5′-3′) | R.E.ª | R.E.^{b} |
| Construction of | R1066F | 1 | GACGGTGTGG | BamHI | BamHI |
| pK18-Ru1066: | | | AGGATCCCATG | | |
| 9 from | | | GAGTGGTACAT | | |
| pK18mobsacBgm | | | TGACTCGG | | |
| | | | (SEQ ID NO. 11) | | |
| | R1066R | 1 | CCTGCCCGTCT | XbaI | — |
| | | | AGAATGGTTAT | | |
| | | | CCGCTCGATCA | | |
| | | | TCGG | | |
| | | | (SEQ ID NO: 12) | | |
| | R1069F | 2 | CCATAGCGGA | SpeI | — |
| | | | CTAGTCAATTA | | |
| | | | CGTCAACCGTA | | |
| | | | TCGGCG | | |
| | | | (SEQ ID NO: 13) | | |
| | R1069R | 2 | CCGCCGCTTGC | SphI | SphI |
| | | | ATGCAAACGC | | |
| | | | CTTGATCCTCA | | |
| | | | AGGC | | |
| | | | (SEQ ID NO: 14) | | |
| Construction of | R0793F | 1 | CTGTTTCAGGA | BamHI | BamHI |
| pK18-Ru0793: | | | TCCTGGGTCCG | | |
| 6 from | | | ACGGTACTCTA | | |
| pK18mobsacBgm | | | TC | | |
| | | | (SEQ ID NO: 15) | | |
| | R0793R | 1 | CCTGACTTTTC | XbaI | — |
| | | | TAGAAAAAAT | | |
| | | | CTACACAACCA | | |
| | | | CCGTCAGCG | | |
| | | | (SEQ ID NO: 16) | | |
| | R0796F | 2 | GAAACTCCGA | SpeI | — |
| | | | CTAGTGCAGGC | | |
| | | | TGGCGGGAAG | | |
| | | | GATAAGC | | |
| | | | (SEQ ID NO: 17) | | |
| | R0796R | 2 | GCGCAAGGGC | SphI | SphI |
| | | | ATGCCGTTGTC | | |
| | | | CATCGTGTATG | | |
| | | | GCG | | |
| | | | (SEQ ID NO: 18) | | |
| Construction of | R0772F | 1 | CAAAGGTGGA | BamHI | BamHI |
| pK18-Ru0772: | | | TCCACAACGCC | | |
| 3 from | | | ACTTTATCCTC | | |
| pK18mobsacBgm | | | CGC | | |
| | | | (SEQ ID NO: 19) | | |
| | R0772R | 1 | CGGCTGTTTCT | XbaI | — |
| | | | AGACGCCATC | | |
| | | | ACCCACAAACT | | |
| | | | CCAG | | |
| | | | (SEQ ID NO: 20) | | |
| | R0773F | 2 | CGTCGTTCGAC | SpeI | — |
| | | | TAGTTCGACCG | | |
| | | | GCTGGAGCGG | | |
| | | | C | | |
| | | | (SEQ ID NO: 21) | | |
| | R0773R | 2 | CCGTATCGGCA | SphI | SphI |
| | | | TGCCAACCCAG | | |
| | | | GACGCCTTTG | | |
| | | | (SEQ ID NO: 22) | | |
| Construction of | C0796F | 1 | GGAGACGGCT | NdeI | NdeI |
| pMTAP-marBH | | | CATATGACGGT | | |
| from pMTAP- | | | TCCTGCTTATC | | |
| MCS3 | | | CTTCCCGC | | |
| | | | (SEQ ID NO: 23) | | |
| | C0795R | 1 | GATGGGCATG | KpnI | KnpI |
| | | | GTACCCGTTAT | | |
| | | | GAGGCCAGG | | |
| | | | (SEQ ID NO: 24) | | |
| Construction of | C0794F1 | 1 | CGGAGCGGCC | NdeI | NdeI |
| pMTAP-marDK | | | ATATGCCCATC | | |
| from pMTAP- | | | AATCTCAAGAC | | |
| MCS3 | | | ATCGGTGG | | |
| | | | (SEQ ID NO: 25) | | |
| | C0793R | 1 | GGCGGCCTCG | XhoI | XhoI |
| | | | AGCCCGGATG | | |
| | | | CCGCCATTCC | | |
| | | | (SEQ ID NO: 26) | | |
| Construction of | C0794F2 | 1 | CGGAGCGGTA | KpnI | KpnI |
| pMTAPmarBH: mar | | | CCATGCCCATC | | |
| DK from pMTAP- | | | AATCTCAAGAC | | |
| marBH | | | ATCGG | | |
| | | | (SEQ ID NO: 27) | | |
| | C0793R | 1 | GGCGGCCTCG | XhoI | XhoI |
| | | | AGCCCGGATG | | |
| | | | CCGCCATTCC | | |
| | | | (SEQ ID NO: 28) | | |
| Construction of | C0773F | 1 | GGAGGCGGGT | KpnI | KpnI |
| pMTAPmarBH: nfl | | | ACCGTGACAA | | |
| DK from pMTAP- | | | AGATCGAAAA | | |
| marBH | | | GCCGCTCCAGC | | |
| | | | C | | |
| | | | (SEQ ID NO: 29) | | |
| | C0772R | 1 | CATCACCCCTC | XhoI | XhoI |
| | | | GAGCCACACC | | |
| | | | GGGCGACCGC | | |
| | | | ACAGC | | |
| | | | (SEQ ID NO: 30) | | |

Gene complementation of the R. rubrum NFL gene deletion strain Δ0772:3/Δ0793:6 was performed in trans by NFL genes expressed from complementation plasmid pMTAP (70). Genes were amplified by PCR using primers listed in Table A, digested with the indicated restriction enzyme, and ligated into pMTAP. Sequence verified plasmids were transformed into E. coli Stellar strain (Takara) and mobilized into R. rubrum by triparental conjugation with helper strain E. coli JM109/pRK2013. Transconjugants were selected on 16% PYE agar plates with 2 μg/ml tetracycline and 50 μg/ml streptomycin under aerobic growth at 30° C. Isolates were then tested for their ability to grow anaerobically with sulfate, MT-EtOH, or DMS as sole sulfur source. R. rubrum Δ0772:3/Δ0793:6 transconjugants with plasmids that complemented for growth on MT-EtOH and DMS were also quantified for restoration of ethylene and methane production by GC as described below.

Whole-cell VOSC utilization and gas production assays: Cells were initially grown aerobically in 150 ml serum bottles containing sulfur-free Ormerod's malate minif al medium supplemented with 50 μM ammonium sulfate (methylthio-alkane reductase inducing conditions) to mid log phase (O.D.₆₆₀ₙₘ of 0.7-0.8). Cultures were washed anaerobically three times by centrifugation and resuspension in sulfur-free Ormerod's malate minimal medium. Cells were resuspended to a final O.D.₆₆₀ₙₘ of ˜2.0 (higher cell densities suppressed methylthio-alkane reductase activity), dispensed in 20 ml aliquots in 60 ml serum vials, fed with 1 mM of DMS, EMS, or MT-EtOH, sealed, and incubated at 30° C. under 2000 lux incandescent illumination for 12 h. Produced methane, ethane, and ethylene gas was quantified by GC as described below.

Whole-cell nitrogenase and methylthio-alkane reductase specific rate assays: R. rubrum wild type and NFL gene deletion (Δ0772:3/Δ0793:6) strains were grown anaerobically under argon headspace to late log phase (O.D.₆₆₀ₙₘ 0.9-1.1) in Ormerod's malate minimal medium with 15 mM ammonium chloride or sodium glutamate as sole N-source and 50 μM or 1 mM sodium sulfate as sole S-source, For whole-cell nitrogenase assays (46), 2 ml of culture was transferred via syringe to an anaerobic 7.5 ml serum vial flushed with argon. Assays were initiated by the addition of 0.06 atm acetylene and allowed to proceed for 10 min under 2000 lux illumination at 30° C. Assays were quenched with 100% (w/v) trichloroacetic acid to 10% final and ethylene was quantified by GC as described below. Similarly, for whole-cell methylthio-alkane reductase assay, 4 ml of culture were transferred via syringe to an anaerobic 8 ml serum vial flushed with argon. Assays were initiated by the addition of EMS to 1 mM final concentration and allowed to proceed for 30 min under 2000 lux illumination at 30° C. Assays were quenched with TCA and ethane was quantified by GC.

GC analysis of hydrocarbons: Quantification of methane, ethane, and ethylene was performed using a Shimazdu GC-14A with Restek Rt-Alumina BOND/Na₂SO₄ column. Gaseous culture headspace after feeding or growth experiments was injected (250-500 μl) at 180° C. and separated isothermally at 30° C. Eluted compounds were detected by flame ionization detector at 180° C. and identified based on retention time of methane, ethane, and ethylene standard (Praxair). The total amount of each hydrocarbon present was calculated from the peak area as compared to standard concentration curves of the corresponding reference standard.

Targeted metabolomics: R. rubrum wild type and Rru_A0793-Ru_A0796 deletion strains were grown anaerobically to an O.D.₆₆₀ₙₘ of 0.8 (mid log phase) in Ormerod's malate minimal medium supplemented with 50 μM ammonium sulfate to induce ethylene production. Cultures were washed anaerobically three times by centrifugation and resuspension in sulfur-free Ormerod's malate minimal medium. Cells were resuspended to a final O.D.₆₆₀ₙₘ of ˜2.0 (higher concentrations repressed methylthio-alkane reductase activity), supplemented with 100 μM 5,5′-dithiobis-(2-nitrobenzoic acid) (Ellman's reagent for trapping free thiols), and sealed as 1 ml aliquots in 1.5 ml anaerobic serum vials. Cells were then fed with 10 μM MT-EtOH and 1 μM (2-[methyl-C¹⁴]thio)ethanol and incubated under 2000 lux incandescent light at 30° C. Metabolism was stopped by flash freezing in liquid nitrogen; cells were pelleted, media supernatant reserved, and the cell pellet was extracted with 80% acetonitrile+0.04N ammonium hydroxide with vortexing for 5 min followed by 20 min incubation at −20° C. Acetonitrile was removed by vacuum concentration, and the extracted metabolites were combined with the reserved media supernatant. Metabolites were separated by reverse phase HPLC and identified by inline scintillation detector based on retention time compared to reference standards as previously described for N=2 biological replicates (10).

Free-energy calculations: Standard free energies of formation and reaction were determined using electronic structure calculations with continuum solvent models. Specifically, density functional theory with the B3YLP (47 , 48) exchange correlation functional was used with the 6-311++G(2d, 2p) basis set. The geometries were optimized and harmonic frequencies determined in a continuum model solvent using the COSMO self-consistent reaction field method (49). All calculations were performed with the NWChem computational chemistry package (50) using the EMSL Arrows interface (5.1). H₂ was used as the electron donor in each redox reaction since the actual electron donor is not known. The relative difference in the reaction free energies will not change if, for example, ferredoxin or any other redox pair were used as the electron donor, since the electrochemical potential of the actual electron donor would be measured relative to the standard hydrogen electrode.

Phylogenetics: The R. rubrum MarH, MarD, and MarK proteins were separately queried against the NCBI reference genome database using the translated nucleotide blast (tblastn) algorithm and filtered for protein subjects with e-value<e-50. Each identified MarH, MarD, and MarK candidate was correlated with its reference genome and only genomes were retained that contained all three homologues on the same contig and with MarD and MarK being adjacent. These candidates, along with recently discovered Group VI representatives from metagenome assembled genomes (28) were then appended to a reference nitrogenase (Groups I, II, III) and NFL sequence (Groups IV and V) database (9) with additional sequences identified from genomes in the JGI IMG/M database. Amino acid sequences were aligned using MAFFT (52) (v7.394) (—auto). Alignments were trimmed using TrimAl (53) (v1.4.rev22) (—gappyont). Maximum likelihood trees were constructed using IQ-TREE (54) (v1.6.8) (−alrt 1000-bb 1000) using best-fit models (NifH: LG+R10; NifD: LG+R6) identified by ModelFinder (55) as implemented in IQ-TREE with ultrafast bootstrap (UFBoot) (56).

Pairwise alignment of NifB, NifH, NifD, and NifK superfamily sequences for conserved active site residue analysis (FIG. 10-FIG. 13) was performed using Clustal Omega (EMBL-EBI) (57) and visualized with Jalview (58). Gene synteny (FIG. 18) was visualized using R package (R Foundation, Vienna, Austria) ‘gggenes’ (59) for an ˜28 kbp neighborhood centered on the NifD homologs identified in selected genomes representing the Nif and NFL clades.

To identify organisms with native ethylene capacity (DHAP Shunt plus marBHDK genes, FIG. 17), organisms with a putative MarHDK complex, as indicated by the phylogenetic tree analysis (FIG. 4 and FIG. 16), were then analyzed for the presence of DHAP shunt homologues by querying each genome (tblastn) with the R. rubrum and E. coli DHAP Shunt genes (10, 25), MtnK, MtnP, MtnA, and Ald2, with a cutoff of e-value <−20. For organism phylogenetic analysis (FIG. 17), 113 genomic sequences including R. rubrum, R. palustris, B. viridis, and additional random organisms with MarHDK genes were downloaded from NCBI (Genome or Assembly databases). This set of genomes was aligned to a set of reference bacteria using GTDB-TK (de_novo_wf) (60). The non-redundant subset of organisms as shown in FIG. 17 together with Chloroflexota sequences as the outgroup from the reference database were extracted from the alignment and a maximum likelihood tree was built using IQ-TREE (54) (−alrt 1000-bb 1000) using the best-fit model LG+F+R6 identified by ModelFinder (55) as implemented in IQ-TREE with ultrafast bootstrap (UFBoot) (56).

### REFERENCES

1. E. E. Stueken, R. Buick, B. M. Guy, M. C. Koehler, Isotopic evidence for biological nitrogen fixation by molybdenum-nitrogenase from 3.2 Gyr. Nature. 520, 666-669 (2015).

2. M, C. Weiss, F. L. Sousa, N. Mrnjavac, S. Neukirchen, M. Roettger, S. Nelson-Sathi, W. F. Martin, The physiology and habitat of the last universal common ancestor. Nat. Microbiol. 1, 16116 (2016).

3. E. S. Boyd, J. W. Peters, New biological insights into the evolutionary history of biological nitrogen fixation. Front. Microbiol. 4, 201 (2013).

4. K. Zheng, P. D. Ngo, V. L. Owens, X. P. Yang, S. O. Mansoorabadi, The biosynthetic pathway of coenzyme F430 in methanogenic and methanotrophic archaea. Science. 354, 339-342 (2016).

5. S. J. Moore, S. T. Sowa, C. Schuchardt, E. Deery, A. D. Lawrence, J. V. Ramos, S. Billig, C. Birkemeyer, P. T. Chivers, M. J. Howard, S. E. Rigby, G. Layer, M. J. Warren, Elucidation of the biosynthesis of the methane catalyst coenzyme F430. Nature. 543, 78-82 (2017).

6. N. Muraki, J. Nomata, K. Ebata, T. Mizoguchi, T. Shiba, H. Tamiaki, G. Kurisu, X. Y. Fujita, X-ray crystal structure of the light-independent protochlorophyllide reductase. Nature. 465, 110-4 (2010).

7. J. INomata, T. Mizoguchi, H. Tamiaki, Y. A. Fujita, A second nitrogenase-like enzyme for bacteriochlorophyll biosynthesis: reconstitution of chlorophyllide a reductase with purified X-protein (BchX) and YZ-protein (BchY-BchZ) from Rhodobacter capsulatus. J. Biol. Chem. 281, 15021-8 (2006).

8. P. C. Dos Santos, Z. Fang, S. W. Mason, J. C. Setubal, R. Dixon, Distribution of nitrogen fixation and nitrogenase-like sequences amongst microbial genomes. BMC Genomics. 13, 162 (2012).

9. J. Raymond, J. L. Siefert, C. R. Stales, R. E. Blankenship, The natural history of nitrogen fixation. Mol. Biol. Evol. 21, 541-54 (2004).

10. J. A. North, A. R. Miller, J. A. Wildenthal, S. J. Young, F. R. Tabita, Microbial pathway for anaerobic 5′-methylthioadenosine metabolism coupled to ethylene formation. Proc. Natl. Acad. Sci. U.S.A. 114, E10455-E10464 (2017).

11. N. Parveen, K. A. Cornell, Methylthioadenosine/S-adenosylhomocysteine nucleosidase, a critical enzyme for bacterial metabolism. Mol. Microbial. 79, 7-20 (2011).

12. S. Burén, E. Jiménez-Vicente, C. Echavarri-Erasun, L. M, Rubio, Biosynthesis of nitrogenase cofactors. Chemical Reviews. doi: 10.1021/acs.chemrev.9b00489 (2020).

13. T. J. Erb, B. S. Evans, K. Cho, B. P. Warlick, J. Sriram, B. M. Wood, H. J. Imker, J. V. Sweedler, F. R. Tabita., J. A. Gerlt, A RuBisCO-like protein links SAM metabolism with isoprenoid biosynthesis. Nat. Chem. Biol. 8, 926-932 (2012).

14. Y. Zhang, E. L. Pohlmann, P. W. Ludden, G. P. Roberts, Mutagenesis and functional characterization of the glnB, glnA, and nifA genes from the photosynthetic bacterium Rhodaspirillum rubrum. J. Bacteriol. 182, 983-92 (2000).

15. Sippel, O. Einsle, The structure of vanadium nitrogenase reveals an unusual bridging ligand. Nat. Chem. Biol. 13, 956-960 (2017).

16. L. M. Zhang, C. M. Morrison, J. T. Kaiser, D. C. Reese, Nitrogenase MoFe protein from Clostridium pasteurianum at 1.08 Å resolution: comparison with the Azotobacter vinelandii MoFe protein. Acta Crystallogr. D Biol. Crystallogr. 71, 274-282 (2015).

17. D, Sippel, M. Rohde, J. Netzer, C. Trncik, J, Gies, K. Grunau, I. Djurdjevic, L. Decamps, S. L. A. Andrade, O. Einsle, A bound reaction intermediate sheds light on the mechanism of nitrogenase. Science. 359, 1484-1489 (2018).

18. M. Bižić, T. Klintzsch, D. Ionescu, M. Y. Hindiyeh, M. Günthel, A. M. Muro-Pastor, W. Eckert, T. Urich, F. Keppler, H.-P. Grossart, Aquatic and terrestrial cyanobacteria produce methane. Sci. Adv. 6, eaax5343 (2020)

19. D. Repeta, S. Ferran, O. Sosa, C. G. Johnson, L. D. Repeta, M. Acker, E. F. DeLong, D. M. Karl, Marine methane paradox explained by bacterial degradation of dissolved organic matter. Nat. Geosci. 9, 884-887 (2016).

20. Y. Zheng, D. F. Harris, Z. Yu, Y. Fu, S. Poudel, R. N. Ledbetter, K. R. Fixen, Z. Y. Yang, E. S. Boyd, M. E. Lidstrom, L. C. Seefeldt, C. S. Harwood, A pathway for biological methane production using bacterial iron-only nitrogenase. Nat. Microbiol. 3, 281-286 (2018).

21. K. A. Smith, R. S. Russell, Occurrence of ethylene and its significance in anaerobic soils. Nature. 222, 769-771 (1969).

22. S. Manik, G. Pengilley, G. Dean, B. Field, S. Shabala, M. Zhou, Soil and crop management practices to minimize the impact of waterlogging on crop productivity. Front. Plant. Sci., 10, 140 (2019).

23. J. M. Lynch, Identification of substrates and isolation of micro-organisms responsible for ethylene production in soil. Nature. 240, 45-46 (1972).

24. J. M, Lynch, Ethylene in soil. Nature. 256, 576-577 (1975).

25. J. A. North, J. A. Wildenthal, T. J. Erb, B. E. Evans, K. M. Byerly, J. A. Gerlt, F. R. Tabita, A bifunctional salvage pathway for two distinct S-adenosylmethionine byproducts that is widespread in bacteria, including pathogenic Escherichia coli. Mol. Microbiol. 10.1111/mmi.14459 (2020).

26. G. A. W. Beaudoin, Q. Li, J. Folz, O. Fiehn, J. L. Goodsell, A. Angerhofer, S. D. Bruner, A. D, Hanson, Salvage of the 5-deoxyribose byproduct of radical SAM enzymes. Nat. Commun. 9, 3105 (2018).

27. H. Zheng, C. Dietrich, R. Radek, A. Brune, Endomicrobium proavitum, the first isolate of Endomicrobia class. nov. (phylum Elusimicrobia)—an ultramicrobacterium with an unusual cell cycle that fixes nitrogen with a Group IV nitrogenase. Environ. Microbiol. 18, 191-204 (2016).

28. R., Méheust, C. J. Castelle, P. B. M. Carnevali, I. F. Farag, C. He, L. X. Chen, Y. Amano, L. A. Hug. J. F. Banfield, Aquatic Elusimicrobia are metabolically diverse compared to gut microbiome Elusimicrobia and some have novel nitrogenase-like gene clusters. https://www.biorxiv.org/content/10.1101/765248v2 (2019).

29. H. J. Imker, A. A. Fedorov, E. V. Fedorov, S. C. Almo, J. A. Gerlt, Mechanistic diversity in the RuBisCO superfamily: the “enolase” in the methionine salvage pathway in Geobacillus kaustophilus. Biochemistry. 46, 4077-89 (2007).

30. J. Singh, F. R. Tabita, Roles of RubisCO and the RubisCO-like protein in 5-methylthioadenosine metabolism in the nonsulfur purple bacterium Rhodospirillum rubrum. J. Bacteriol. 192, 1324-31 (2010).

31. H. Strnad, A. Lapidus, J. Paces, P. Ulbrich, C. Vlcek, V. Paces, R. Haselkorn, Complete genome sequence of the photosynthetic purple nonsulfur bacterium Rhodobacter capsulatus SB 1003. J. Bacteriol. 192, 3545-6 (2010).

32. F. E. Rey, Y. Oda, C. S. Harwood, Regulation of uptake hydrogenase and effects of hydrogen utilization on gene expression in Rhodopseudomonas palustris. J. Bacteriol. 188, 6143-6152 (2006).

33. G. Drews, P. Giesbrecht, Rhodopseudomonas viridis, nov. spec., ein neu isoliertes, obligat phototrophes Bakterium. Archiv für Mikrobiol. 53, 255-262 (1966).

34. J. G. Ormerod, K. S. Ormerod, H. Gest, Light-dependent utilization of organic compounds and photoproduction of molecular hydrogen by photosynthetic bacteria; relationships with nitrogen metabolism, Arch. Biochem. Biophys. 94, 449-463 (1961).

35. S. Dey, J. A. North, J. Sriram, B. S. Evans, F. R. Tabita, In vivo studies in Rhodospirillum rubrum indicate that ribulose-1,5-bisphosphate carboxylase/oxygenase (Rubisco) catalyzes two obligatorily required and physiologically significant reactions for distinct carbon and sulfur metabolic pathways. J. Biol. Chem. 290, 30658-68 (2015).

36. D. P. Canniffe, D. A. Bryant, Engineered biosynthesis of bacteriochlorophyll b in Rhodobacter sphaeroides. Biochim. Biochim. Acta. 1837, 1611-6 (2014).

37. D. L. Tabb, C. G. Fernando, M. C. Chambers, MyriMatch: highly accurate tandem mass spectral peptide identification by multivariate hypergeometric analysis. J. Proteome Res. 6, 654-661 (2007).

38. Z. Q. Ma, S. Dasari, M. C. Chambers, M. D. Litton, S. M. Sobecki, L. J. Zimmerman, P. J. Halvey, B. Schilling, P. M. Drake, B. W. Gibson, D. L. Tabb, IDPicker 2.0: Improved protein assembly with high discrimination peptide identification filtering. J. Proteome Res. 8, 3872-3881 (2009).

39. T. Taverner, Y. V. Karpievitch, A. D. Polpitiya, J. N. Brown, A. R. Dabney, G. A. Anderson, R. D. Smith, DanteR: an extensible R-based tool for quantitative analysis of omics data. Bioinformatics. 28, 2404-2406 (2012).

40. S. Tyanova, T. Temu, P. Sinitcyn, A. Carlson, M. Y. Hein, T. Geiger, M. Mann, J. Cox, The Perseus computational platform for comprehensive analysis of (prote)omics data. Nat. Methods. 9, 731-740 (2016).

41. N. A. Joshi, J. N. Fass, Sickle: A sliding-window, adaptive, quality-based trimming tool for FastQ files (Version 1.33) https://github.com/najoshi/sickle (2011).

42. B. Langmead, S. L. Salzberg, Fast gapped-read alignment with Bowtie 2. Nat. Methods. 9, 357-359 (2012).

43. M. I. Love, W. Huber, S. Anders, Moderated estimation of fold change and dispersion for RNA-Seq data with DESeq2. Genome Biol. 15, 550 (2014).

44. R. A. Larsen, M. M. Wilson, A. M. Guss, W. W. Metcalf, Genetic analysis of pigment biosynthesis in Xanthobacter autotrophicus Py2 using a new, highly efficient transposon mutagenesis system that is functional in a wide variety of bacteria. Arch. Microbiol. 178, 193-201 (2002).

45. D. H. Figurski, D. R. Helinski, Replication of an origin-containing derivative of plasmid RK2 dependent on a plasmid function provided in trans. Proc. Natl. Acad. Sci. U.S.A. 76, 1648-1652 (1979).

46. R. W. F. Hardy, R. D. Holsten, E. K. Jackson, R. C. Burns, The acetylene reduction assay for N₂ fixation: laboratory and field evaluation, Plant Physiol. 43, 1185-1207 (1968).

47. C. T. Lee, W. T. Yang, R. G. Parr, Development of the Colle-Salvetti correlation-energy formula into a functional of the electron-density. Phys. Rev. B. 37, 785-789 (1988).

48. A. D. Becke, Density-functional thermochemistry. III. The role of exact exchange. J. Chem. Phys. 98, 5648-5652 (1993).

49. A. Klamt, G. Schuurmann, Cosmo—a new approach to dielectric screening in solvents with explicit expressions for the screening energy and its gradient. J. Chem. Soc., Perkin Trans. 2. 1993, 799-805 (1993).

50. M. Valiev, E. J. Bylaskaa, N. Govinda, K. Kowalskia, T. P. Straatsmaa, H. J. J. Van Dama, D. Wanga, J. Nieplochaa, E. Aprab, T. L. Windusc, W. A. de Jonga, NWChem: A comprehensive and scalable open-source solution for large scale molecular simulations. Comput. Phys. Commun. 181, 1477-1489 (2010).

51. E. J. Bylaska, EMSL Arrows. https://arrows.emsl.pnnl.gov/api/ (2020).

52. K. Katoh, D. M. Standley, MAFFT multiple sequence alignment software version 7: improvements in performance and usability. Mol. Biol. Evol. 30, 772-780 (2013).

53. S. Capella-Gutierrez, J. M. Silla-Martinez, I. Gabaldon, TrimAl: a tool for automated alignment trimming in large-scale phylogenetic analyses. Bioinformatics. 25, 1972-1973 (2009),

54. L. T. Nguyen, H. A. Schmidt, A. von Haeseler, B. Q. Minh, IQ-TREE: a fast and effective stochastic algorithm for estimating maximum-likelihood phylogenies. Mol. Biol. Evol. 32, 268-274 (7 1015).

55. S. Kalyaanamoorthy, B. Q. Minh, T. K. F. Wong, A. von Haeseler, L. S. Jermiin, ModelFinder: fast model selection for accurate phylogenetic estimates. Nat. Methods. 14, 587-589 (2017).

56. D. T. Hoang, O. Chernomor, A. von Haeseler, B. Q. Minh, L. S. Vinh, UFBoot2: Improving the ultrafast bootstrap approximation. Mol. Biol. Evol. 35, 518-522 (2018).

57. F. Madeira, Y. M. Park, J. Lee, N. Buso, T. Gur, N. Madhusoodanan, P. Basutkar, A. R. N. Tivey, S. C. Potter, R. D. Finn, R. Lopez, The EMBL-EBI search and sequence analysis tools APIs in 2019. Nucleic Acids. Res. 47, W636-W641 (2019).

58. A. M. Waterhouse, J. B. Procter, D. M. A. Martin, M. Clamp, G. J. Barton, Jalview Version 2—a multiple sequence alignment editor and analysis workbench. Bioinformatics. 25, 1189-1191 (2009).

59. D. Wilkins, gggenes: Draw Gene Arrow Maps in ‘ggplot2’. R package version 0.4.0. https://wilkox.org/gggenes (2019).

60. P.-A. Chaumeil, A. J. Mussig, P. Hugenholtz, D. H. Parks, GTDB-Tk: a toolkit to classify genomes with the Genome Taxonomy Database. Bioinformatics 36, 1925-1927 (2019).

61. S. Poudel, D. R. Colman, K. R, Fixen, R. N. Ledbetter, Y. Zheng, N. Pence, L. C. Seefeldt, J. W. Peters, C. S. Harwood, E. S. Boyd. Electron Transfer to Nitrogenase in Different Genomic and Metabolic Backgrounds. J. Bacteriol. 200, e00757-17 (2018).

62. B. M. Hoffman, D. Lukoyanov, Z-.Y. Yang, D. R. Dean, L. C. Seefeldt, Mechanism of nitrogen fixation by nitrogenase: the next stage. Chem. Rev. 114, 4041-62 (2014).

63. J. Oetjen, B. Reinhold-Hurek, Characterization of the DraT/DraG system for posttranslational regulation of nitrogenase in the endophytic betaproteobacterium Azoarcus sp. Strain BH72. J. Bacteriol. 191, 3726-3735 (2009).

64. M. J. Bröcker, S. Virus, S. Ganskow, P. Heathcote, D. W. Heinz, W. D. Schubert, D. Jahn, J. Moser, ATP-driven reduction by dark-operative protochlorophyllide oxidoreductase from Chlorobium tepidum mechanistically resembles nitrogenase catalysis. J. Biol. Chem. 283, 10559-67 (2008).

65. S. J. Moore, S. I. Sowa, C. Schuchardt, E. Deery, A. D. L., J. Vazquez Ramos, S. Billig, C. Birkemeyer, P. T. Chivers, M. J. Howard, S. E. J. Rigby, G. Layer, M. J., Warren Elucidation of the biosynthesis of the methane catalyst coenzyme F430, Nature. 543, 78-82 (2017).

66. Y. Hu, J. M. Yoshizawa, A. W. Fay, C. Chung Lee, J. A. Wiig, M. W. Ribbe, Catalytic activities of NifEN: Implications for nitrogenase evolution and mechanism. Proc. Natl. Acad. Sci. U.S.A. 106, 16962-16966 (2009).

67. Miller, A. R., North, J. A., Wildenthal, J. A. & Tabita, F. R. Two distinct aerobic methionine salvage pathways generate volatile methanethiol in Rhodopseudomonas palustris. MBio 9, e00407-18 (2018).

68. Varaljay, V. A., Satagopan, S., North, J. A., Witte, B., Dourado, M. N., Anantharaman, K., Arbing, M. A., Hoeft McCann, S., Oremland, R. S., Banfield, J. F., Wrighton, K. C. and Tabita, F. R. Functional metagenomic selection of RubisCO from uncultivated bacteria. Environ. Microbiol. 18, 1187-1199 (2016).

69. J. J. Hultqvist, O. Warsi, A. Söderholm, M. Knopp, U. Eckhard, E. Vorontsov, M. Selmer, D. A. Andersson. A bacteriophage enzyme induces bacterial metabolic perturbation that confers a novel promiscuous function. Nat Ecol Evol. 2, 1321-1330 (2018).

70. J. A. Hughes. In vivo hydrolysis of S-adenosyl-L-methionine in Escherichia coli increases export of 5-methylthioribose. Can J Microbiol, 52, 599-602 (2006).

71. Curson, A. R. J., Todd, J. D., Sullivan, M. J. & Johnston, A. W. B. Catabolism of dimethylsulphoniopropionate: microorganisms, enzymes and genes. Nat. Rev. Microbiol. 9, 849-859 (2011).

72. Carrión, O., Curson, A., Kumaresan, D., Fu, Y., Lang, A. S., Mercadé, E. & Todd, J. D. A novel pathway producing dimethylsulphide in bacteria is widespread in soil environments. Nat Commun 6, 6579 (7 1015).

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A non-naturally occurring microbial organism comprising a nucleic acid encoding one or more genes of a methylthio-alkane reductase complex and one or more genes of a methionine salvage pathway.

2. The non-naturally occurring microbial organism of claim 1, wherein the organism produces ethylene, ethane, methane, or combinations thereof.

3-5. (canceled)

6. The non-naturally occurring microbial organism of claim 1, wherein the one or more genes of a methylthio-alkane reductase complex comprise marB, marH, marD, marK, or combinations thereof.

7. The non-naturally occurring microbial organism of claim 1, wherein the one or more genes of a methylthio-alkane reductase complex comprise a nucleic acid sequence having at least 85% identity to the nucleic acid sequence of SEQ ID NO: 1.

8. (canceled)

9. The non-naturally occurring microbial organism of claim 1, wherein the one or more genes of a methylthio-alkane reductase complex comprise a nucleic acid sequence having at least 85% identity to the nucleic acid sequence of SEQ ID NO: 3.

10. (canceled)

11. The non-naturally occurring microbial organism of claim 1, wherein the one or more genes of a methylthio-alkane reductase complex comprise a nucleic acid sequence having at least 85% identity to the nucleic acid sequence of SEQ ID NO: 5.

12. (canceled)

13. The non-naturally occurring microbial organism of claim 1, wherein the one or more genes of a methylthio-alkane reductase comprise a nucleic acid sequence having at least 85% identity to the nucleic acid sequence of SEQ ID NO: 7.

14. (canceled)

15. The non-naturally occurring organism of any onc of claim 1, wherein the one or more genes of a methionine salvage pathway comprise one or more genes of a dihydroxyacetone phosphate (DHAP) shunt pathway.

16. The non-naturally occurring organism of claim 15, wherein the one or more genes of a DHAP shunt pathway comprise 5′-methylthioadenosine phosphorylase (mtnP), methylthioadenosine nucleosidase (mtnl), 5-methylthioribose kinase (mtnK), 5-methylthioribose-1-phosphate isomerase (mtnA), 5-methylthioribulose-1-phosphate aldolase (ald2), or combinations thereof.

17-20. (canceled)

21. The non-naturally occurring microbial organism of claim 1, wherein the nucleic acid further encodes one or more genes of a SAM hydrolase, or
wherein the nucleic acid further encodes one or more genes of a methanethiol methylase (mddA), a methionine gamma lyase (mgl), or combinations thereof.

22. (canceled)

23. (canceled)

24. The non-naturally occurring microbial organism of claim 1, wherein the nucleic acid is integrated into the genome of the organism or is episomally integrated into a plasmid.

25. (canceled)

26. A non-naturally occurring microbial organism, wherein the organism is an anaerobic organism which produces ethylene, ethane, and/or methane using a methylthio-alkane reductase complex and a methionine salvage pathway, and wherein the organism has been optimized for producing ethylene, ethane, and/or methane with one or more non-naturally occurring genes.

27-31. (canceled)

32. A method of producing ethylene, ethane, and/or methane comprising:
culturing a population of the non-naturally occurring microbial organism of claim 1 in a culture medium comprising one or more carbon sources; and
recovering the ethylene, ethane, and/or methane.

33. The method of claim 32, wherein the one or more carbon sources comprise carbon dioxide, carbon monoxide, an organic acid, a volatile fatty acid, an alcohol, cellulosic plant mass, or combinations thereof.

34. The method of claim 32, wherein the one or more carbon sources comprise carbon dioxide, carbon monoxide, malate, succinate, pyruvate, fumarate, formate, acetate, propionate, butyrate, ethanol, glycerol, corn stover, miscanthus, or switchgrass.

35. (canceled)

36. The method of claim 32, wherein the one or more carbon sources comprise lignocellulosic biomass.

37. The method of any one of claim 3, wherein the population is cultured in the absence of sulfate.

38. A bioreactor comprising the non-naturally occurring microbial organism of claim 1.

39. A vector comprising: one or more exogenous nucleic acid molecules encoding one or more genes of a methylthio-alkane reductase complex and one or more genes of a methionine salvage pathway.

40-63. (canceled)

64. A non-naturally occurring organism comprising a vector of claim 39.
